(19) United States
(12) Patent Application Publication
(10) Pub. No.: US 2024/0172561 A1
(43) Pub. Date: May 23, 2024

(54) COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si, Gyeonggi-do (KR)

(72) Inventors: Jonghoon KIM, Suwon-si, Gyeonggi-do (KR); Youngmook LIM, Suwon-si, Gyeonggi-do (KR); Hyung Sun KIM, Suwon-si, Gyeonggi-do (KR); Sung-Hyun JUNG, Suwon-si, Gyeonggi-do (KR); Ho Kuk JUNG, Suwon-si, Gyeonggi-do (KR); Dalho HUH, Suwon-si, Gyeonggi-do (KR)

(21) Appl. No.: 18/279,286

(22) PCT Filed: Mar. 21, 2022
(86) PCT No.: PCT/KR2022/003903

§ 371 Date: Aug. 29, 2023

(30) Foreign Application Priority Data
Mar. 19, 2021 (KR) 10-2021-0036147

(51) Int. Cl. H10K 85/60; C07D 251/24; C07D 491/14; C09K 11/06

(52) U.S. Cl. CPC H10K 85/657; C07D 251/24; C07D 491/14; C09K 11/06; H10K 85/622; H10K 85/654; H10K 50/11

(57) ABSTRACT

Disclosed are a composition for an organic optoelectronic device, an organic optoelectronic device including the same, and a display device. The composition includes a first compound represented by a combination of Chemical Formula 1 and Chemical Formula 2 and a second compound represented by Chemical Formula 3.

Details of Chemical Formula 1 to Chemical Formula 3 are as defined in the specification.

### TECHNICAL FIELD

A composition for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is a light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a composition for an organic optoelectronic device capable of realizing an organic optoelectronic device having high efficiency and long life-span.

Another embodiment provides an organic optoelectronic device including the composition for an organic optoelectronic device.

Another embodiment provides a display device including the organic optoelectronic device.

### Technical Solution

According to an embodiment, a composition for an organic optoelectronic device includes a first compound represented by a combination of Chemical Formula 1 and Chemical Formula 2 and a second compound represented by Chemical Formula 3.

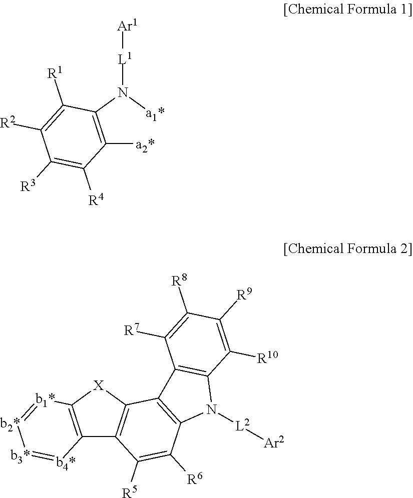

In Chemical Formula 1 and Chemical Formula 2,
a1* and a2* are each linking carbon (C),
b1* to b4* are each independently CR^{a} or linking carbon (C),
a1* and a2* are each linked to adjacent two of b1* to b4*,
R^{a} and R¹ to R¹⁰ are each independently hydrogen, deuterium, a cyano group, a halogen, a substituted or unsubstituted C1 to C30 alkyl group, or a substituted or unsubstituted C6 to C30 aryl group,
L¹ and L² are each independently a single bond or a substituted or unsubstituted C6 to C20 arylene group,
Ar¹ and Ar² are each independently a substituted or unsubstituted C6 to C30 aryl group, and
X is O or S;

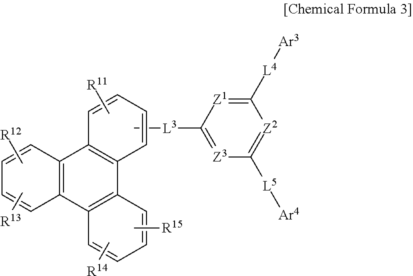

wherein, in Chemical Formula 3,
Z¹ to Z³ are each independently N or C-L^{b}-R^{b},
at least two of Z¹ to Z³ are N,
L^{b} and L³ to L⁵ are each independently a single bond or a substituted or unsubstituted C6 to C20 arylene group,
Ar³ and Ar⁴ are each independently a substituted or unsubstituted C6 to C30 aryl group or a substituted or unsubstituted C2 to C30 heterocyclic group, and
R^{b} and R¹¹ to R¹⁵ are each independently hydrogen, deuterium, a cyano group, a halogen, a substituted or unsubstituted C1 to C30 alkyl group, or a substituted or unsubstituted C6 to C30 aryl group.

According to another embodiment, an organic optoelectronic device includes an anode and a cathode facing each other, and at least one organic layer between the anode and the cathode, wherein the organic layer includes the aforementioned composition for an organic optoelectronic device.

According to another embodiment, a display device including the organic optoelectronic device is provided.

### Advantageous Effects

An organic optoelectronic device having high efficiency and a long life-span may be realized.

### DESCRIPTION OF THE DRAWINGS

An organic optoelectronic device having high efficiency and a long life-span may be realized.

### DESCRIPTION OF SYMBOLS

100: organic light emitting diode
105: organic layer
110: cathode
120: anode
130: light emitting layer
140: hole transport region
150: electron transport region

### MODE FOR INVENTION

Hereinafter, embodiments of the present invention are described in detail. However, these embodiments are exemplary, the present invention is not limited thereto and the present invention is defined by the scope of claims.

In the present specification, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present invention, the “substituted” refers to replacement of at least one hydrogen of a substituent or a compound by deuterium, a C1 to C20 alkyl group, a C6 to C30 aryl group, or a cyano group. In specific example of the present invention, the “substituted” refers to replacement of at least one hydrogen of a substituent or a compound by deuterium, a C1 to C5 alkyl group, a C6 to C18 aryl group, or a cyano group. In specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In the present specification, when a definition is not otherwise provided, “hetero” refers to one including one to three heteroatoms selected from N, O, S, P, and Si, and remaining carbons in one functional group.

In the present specification, “an aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

In the present specification, “a heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, or a combination thereof, but is not limited thereto.

More specifically, the substituted or unsubstituted C2 to C30 heterocyclic group may be a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof, but is not limited thereto.

In the present specification, “hydrogen substitution (-H)” may include “deuterium substitution (-D)” or “tritium substitution (-T).”

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electronic characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a composition for an organic optoelectronic device according to an embodiment is described.

The composition for an organic optoelectronic device according to an embodiment includes a first compound represented by a combination of Chemical Formula 1 and Chemical Formula 2 and a second compound represented by Chemical Formula 3.

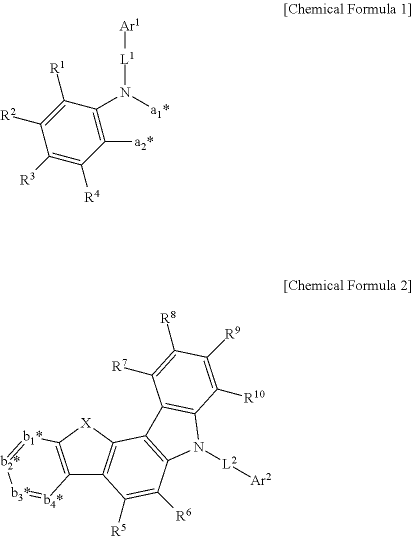

In Chemical Formula 1 and Chemical Formula 2,
a1* and a2* are each linking carbon (C),
b1* to b4* are each independently CR^{a} or linking carbon (C),
a1* and a2* are each linked to adjacent two of b1* to b4*,
R^{a} and R¹ to R¹⁰ are each independently hydrogen, deuterium, a cyano group, a halogen, a substituted or unsubstituted C1 to C30 alkyl group, or a substituted or unsubstituted C6 to C30 aryl group,
L¹ and L² are each independently a single bond or a substituted or unsubstituted C6 to C20 arylene group,
Ar¹ and Ar² are each independently a substituted or unsubstituted C6 to C30 aryl group, and
X is O or S;

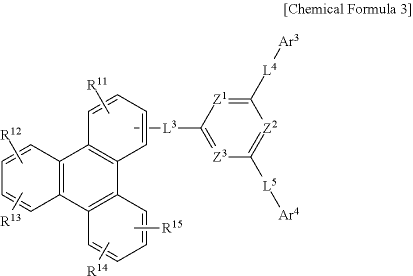

wherein, in Chemical Formula 3,
Z¹ to Z³ are each independently N or C-L^{b}-R^{b},
at least two of Z¹ to Z³ are N,
L^{b} and L³ to L⁵ are each independently a single bond or a substituted or unsubstituted C6 to C20 arylene group,
Ar³ and Ar⁴ are each independently a substituted or unsubstituted C6 to C30 aryl group or a substituted or unsubstituted C2 to C30 heterocyclic group, and
R^{b} and R¹¹ to R¹⁵ are each independently hydrogen, deuterium, a cyano group, a halogen, a substituted or unsubstituted C1 to C30 alkyl group, or a substituted or unsubstituted C6 to C30 aryl group.

The first compound has a structure in which two carbazoles are fused through a furan or thiophene, and has a plate-like structure in which rotation is impossible, unlike conventional bicarbazole compounds capable of rotation around a single bond.

The compound having such a fused structure has improved hole transport characteristics, and accordingly, hole characteristics of an organic light emitting diode to which it is applied may be enhanced, thereby realizing low driving characteristics.

On the other hand, the second compound has a structure in which pyrimidine or triazine is substituted in the triphenylene skeleton, and electron transport characteristics are improved, and electronic characteristics of an organic light emitting diode to which it is applied can be enhanced. Accordingly, by using in combination with the aforementioned first compound, the movement characteristics of holes and electrons are balanced, so that low driving, high efficiency, and long life-span characteristics can be implemented.

For example, the first compound may be represented by any one of Chemical Formula 1A to Chemical Formula 1F.

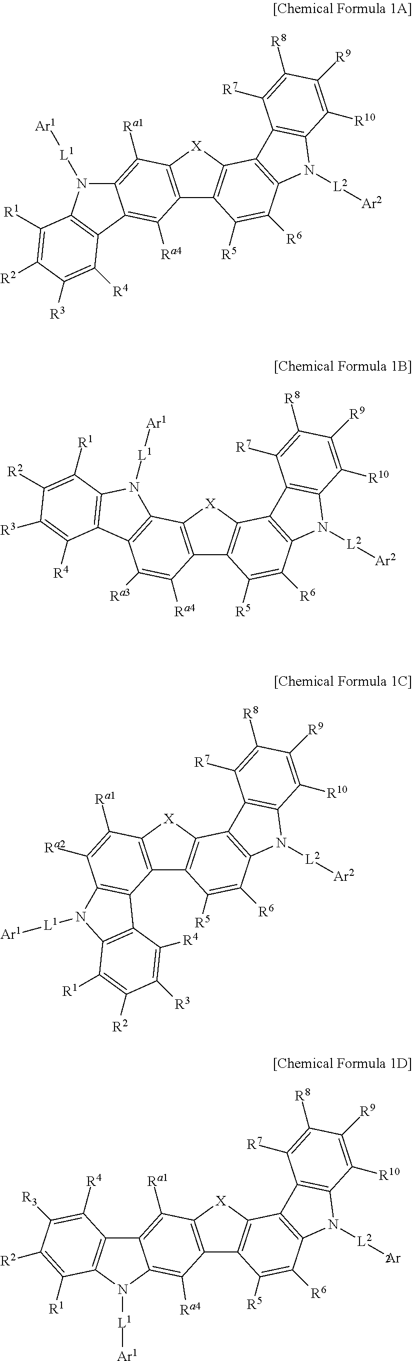

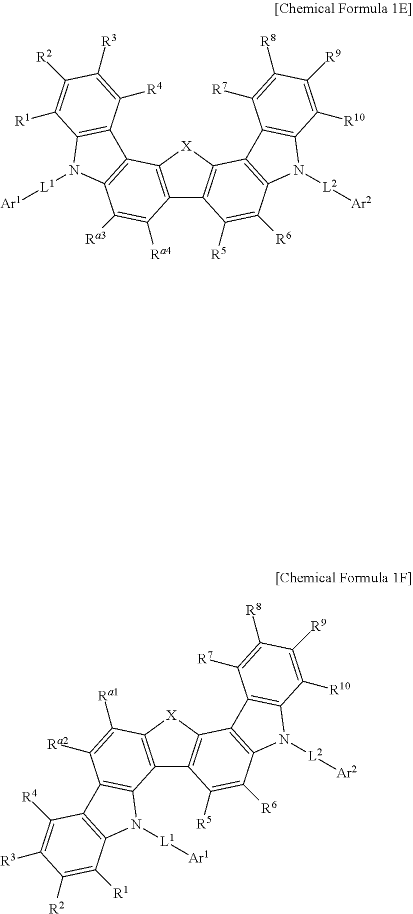

In Chemical Formula 1A to Chemical Formula 1F,
R¹ to R¹⁰, L¹, L², Ar¹, Ar², and X are the same as described above, and
R^{a1} to R^{a4} are each independently the same as the definition of R^{a}.

In an embodiment, the first compound may be represented by Chemical Formula 1A or Chemical Formula 1B.

In a specific embodiment, the first compound may be represented by Chemical Formula 1B.

For example, Ar¹ and Ar² may each independently be a substituted or unsubstituted phenyl group, a substituted or unsubstituted biphenyl group, or a substituted or unsubstituted naphthyl group.

As a specific example, Ar¹ and Ar² may each independently be a substituted or unsubstituted phenyl group or a substituted or unsubstituted biphenyl group.

For example, Ar¹ and Ar² may each independently be a substituted or unsubstituted phenyl group, or a substituted or unsubstituted biphenyl group, and at least one of Ar¹ and Ar² may be a substituted or unsubstituted biphenyl group.

For example, Ar¹ and Ar² may each independently be selected from the substituents listed in Group I.

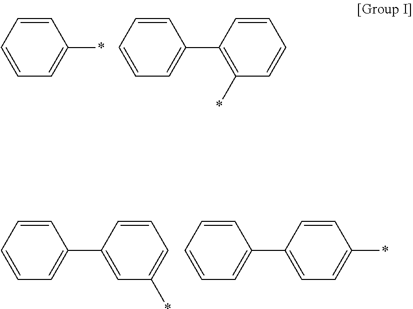

In Group I, * is a linking point.

For example, L¹ and L² may each independently be a single bond or a substituted or unsubstituted phenylene group.

For example, R¹ to R¹⁰ and R^{a1} to R^{a4} may each independently be hydrogen, deuterium, a cyano group, a halogen group, a substituted or unsubstituted C1 to C10 alkyl group, or a substituted or unsubstituted C6 to C12 aryl group.

As a specific example, R¹ to R¹⁰ and R^{a1} to R^{a4} may each independently be hydrogen, deuterium, a substituted or unsubstituted C1 to C5 alkyl group, a substituted or unsubstituted phenyl group, a substituted or unsubstituted biphenyl group, or a substituted or unsubstituted naphthyl group.

For example, each of R¹ to R¹⁰ and R^{a1} to R^{a4} may be hydrogen.

For example, the first compound may be one selected from compounds listed in Group 1.

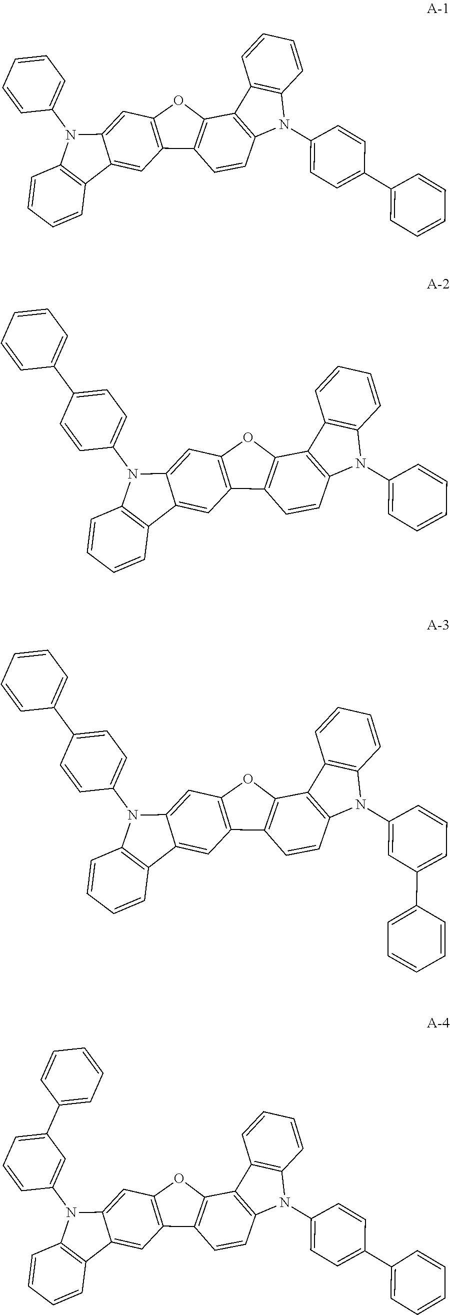 [SMILES: c1ccc(-c2ccc(-n3c4ccccc4c4c5oc6cc7c(cc6c5ccc43)c3ccccc3n7-c3ccccc3)cc2)cc1 ; c1ccc(-c2ccc(-n3c4ccccc4c4cc5c(cc43)oc3c5ccc4c3c3ccccc3n4-c3ccccc3)cc2)cc1 ; c1ccc(-c2ccc(-n3c4ccccc4c4cc5c(cc43)oc3c5ccc4c3c3ccccc3n4-c3cccc(-c4ccccc4)c3)cc2)cc1 ; c1ccc(-c2ccc(-n3c4ccccc4c4c5oc6cc7c(cc6c5ccc43)c3ccccc3n7-c3cccc(-c4ccccc4)c3)cc2)cc1]

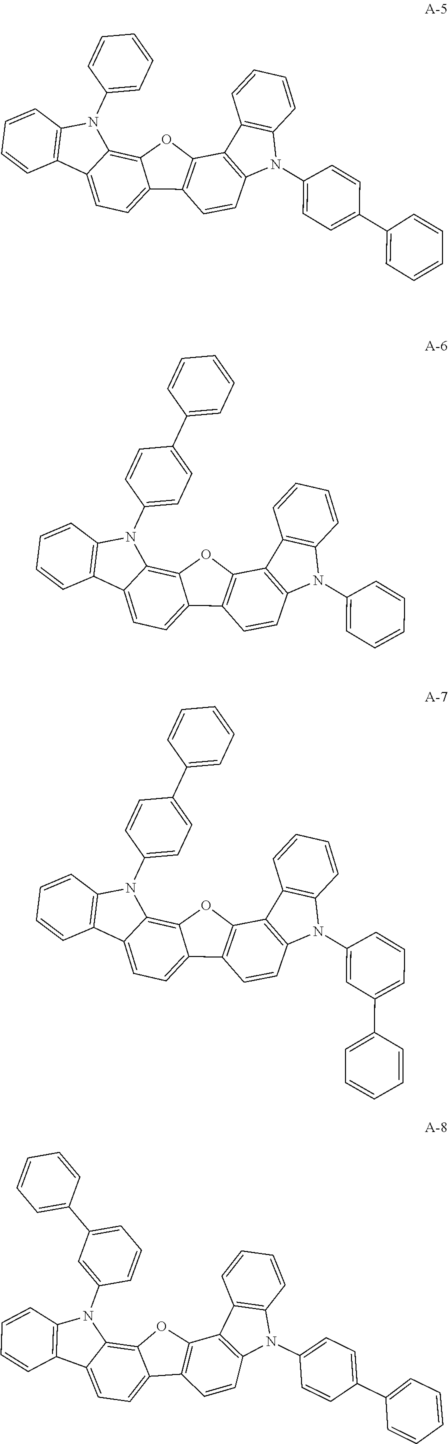 [SMILES: c1ccc(-c2ccc(-n3c4ccccc4c4c5oc6c(ccc7c8ccccc8n(-c8ccccc8)c76)c5ccc43)cc2)cc1 ; c1ccc(-c2ccc(-n3c4ccccc4c4ccc5c6ccc7c(c8ccccc8n7-c7ccccc7)c6oc5c43)cc2)cc1 ; c1ccc(-c2ccc(-n3c4ccccc4c4ccc5c6ccc7c(c8ccccc8n7-c7cccc(-c8ccccc8)c7)c6oc5c43)cc2)cc1 ; c1ccc(-c2ccc(-n3c4ccccc4c4c5oc6c(ccc7c8ccccc8n(-c8cccc(-c9ccccc9)c8)c76)c5ccc43)cc2)cc1]

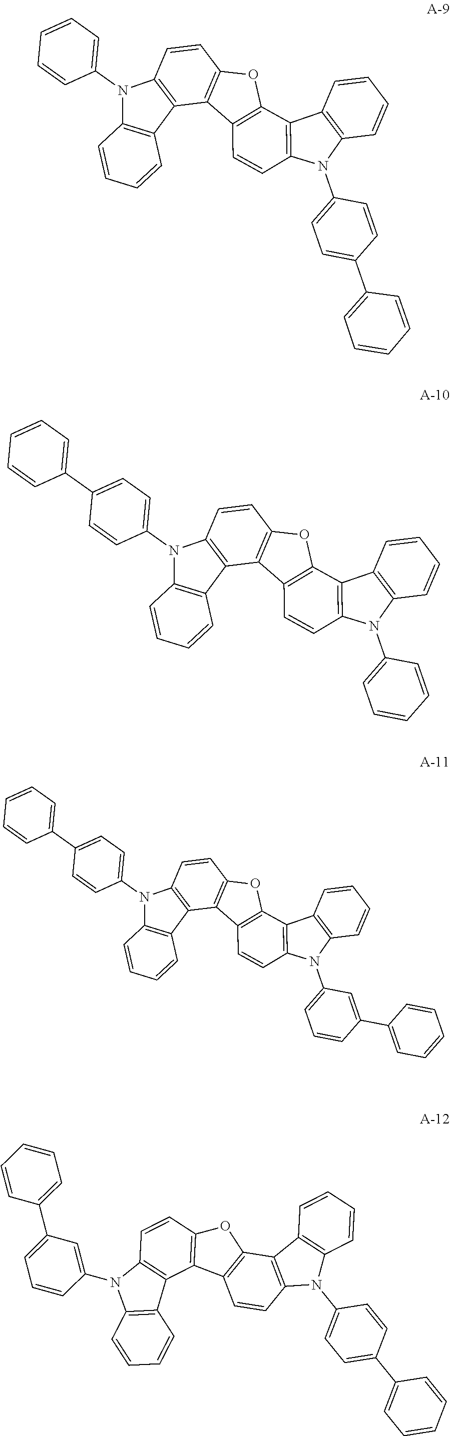 [SMILES: c1ccc(-c2ccc(-n3c4ccccc4c4c5oc6ccc7c(c8ccccc8n7-c7ccccc7)c6c5ccc43)cc2)cc1 ; c1ccc(-c2ccc(-n3c4ccccc4c4c5c(ccc43)oc3c5ccc4c3c3ccccc3n4-c3ccccc3)cc2)cc1 ; c1ccc(-c2ccc(-n3c4ccccc4c4c5c(ccc43)oc3c5ccc4c3c3ccccc3n4-c3cccc(-c4ccccc4)c3)cc2)cc1 ; c1ccc(-c2ccc(-n3c4ccccc4c4c5oc6ccc7c(c8ccccc8n7-c7cccc(-c8ccccc8)c7)c6c5ccc43)cc2)cc1]

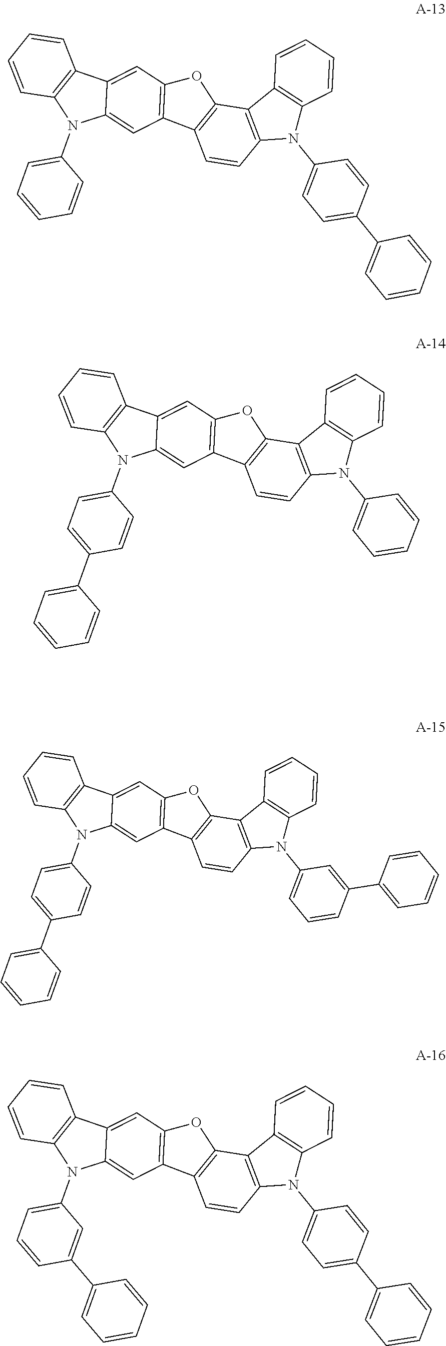 [SMILES: c1ccc(-c2ccc(-n3c4ccccc4c4c5oc6cc7c8ccccc8n(-c8ccccc8)c7cc6c5ccc43)cc2)cc1 ; c1ccc(-c2ccc(-n3c4ccccc4c4cc5oc6c(ccc7c6c6ccccc6n7-c6ccccc6)c5cc43)cc2)cc1 ; c1ccc(-c2ccc(-n3c4ccccc4c4cc5oc6c(ccc7c6c6ccccc6n7-c6cccc(-c7ccccc7)c6)c5cc43)cc2)cc1 ; c1ccc(-c2ccc(-n3c4ccccc4c4c5oc6cc7c8ccccc8n(-c8cccc(-c9ccccc9)c8)c7cc6c5ccc43)cc2)cc1]

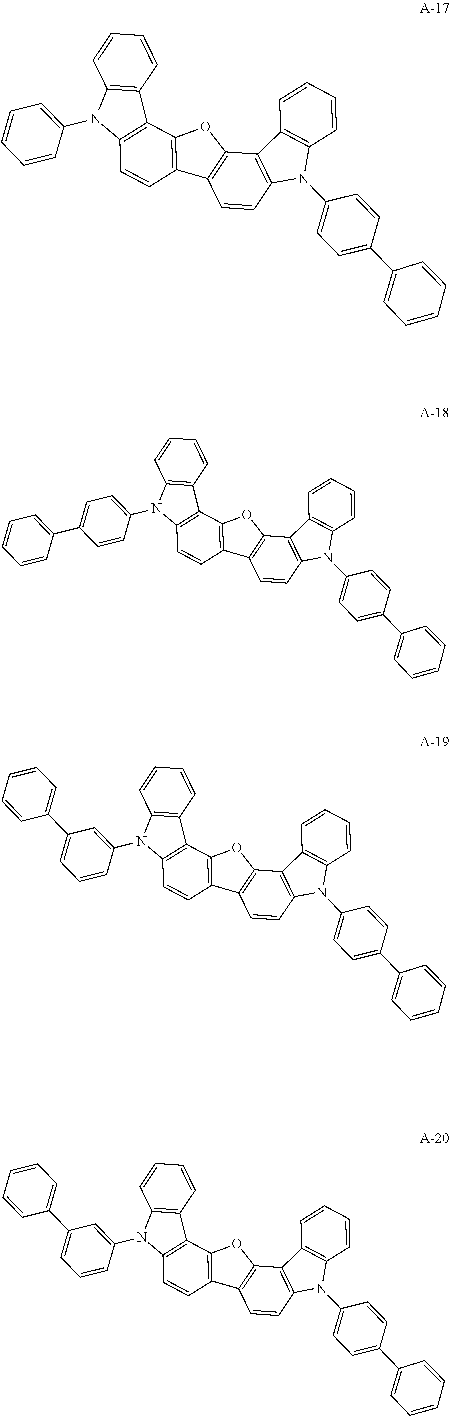 [SMILES: c1ccc(-c2ccc(-n3c4ccccc4c4c5oc6c(ccc7c6c6ccccc6n7-c6ccccc6)c5ccc43)cc2)cc1 ; c1ccc(-c2ccc(-n3c4ccccc4c4c5oc6c(ccc7c6c6ccccc6n7-c6ccc(-c7ccccc7)cc6)c5ccc43)cc2)cc1 ; c1ccc(-c2ccc(-n3c4ccccc4c4c5oc6c(ccc7c6c6ccccc6n7-c6cccc(-c7ccccc7)c6)c5ccc43)cc2)cc1 ; c1ccc(-c2ccc(-n3c4ccccc4c4c5oc6c(ccc7c6c6ccccc6n7-c6cccc(-c7ccccc7)c6)c5ccc43)cc2)cc1]

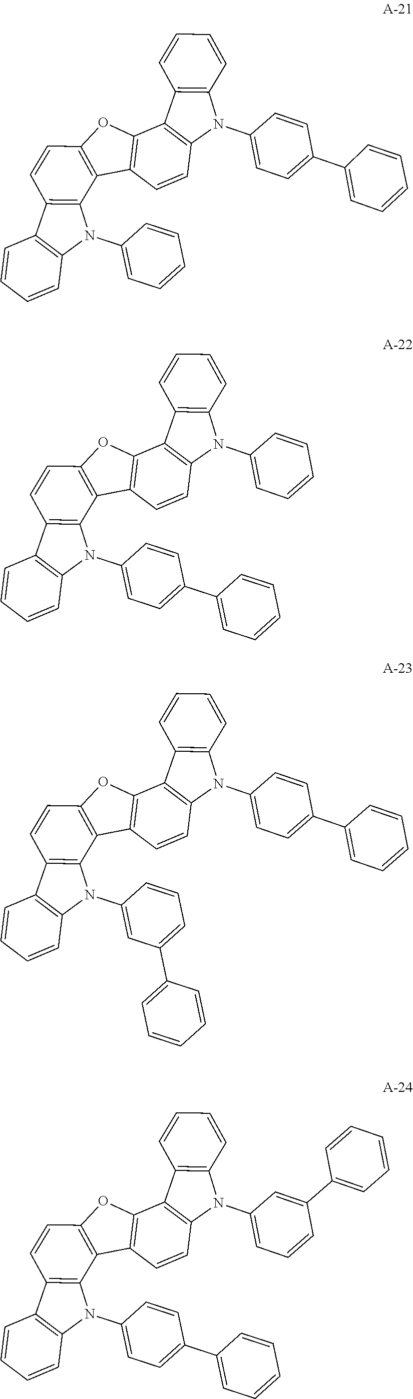 [SMILES: c1ccc(-c2ccc(-n3c4ccccc4c4c5oc6ccc7c8ccccc8n(-c8ccccc8)c7c6c5ccc43)cc2)cc1 ; c1ccc(-c2ccc(-n3c4ccccc4c4ccc5oc6c(ccc7c6c6ccccc6n7-c6ccccc6)c5c43)cc2)cc1 ; c1ccc(-c2ccc(-n3c4ccccc4c4c5oc6ccc7c8ccccc8n(-c8cccc(-c9ccccc9)c8)c7c6c5ccc43)cc2)cc1 ; c1ccc(-c2ccc(-n3c4ccccc4c4ccc5oc6c(ccc7c6c6ccccc6n7-c6cccc(-c7ccccc7)c6)c5c43)cc2)cc1]

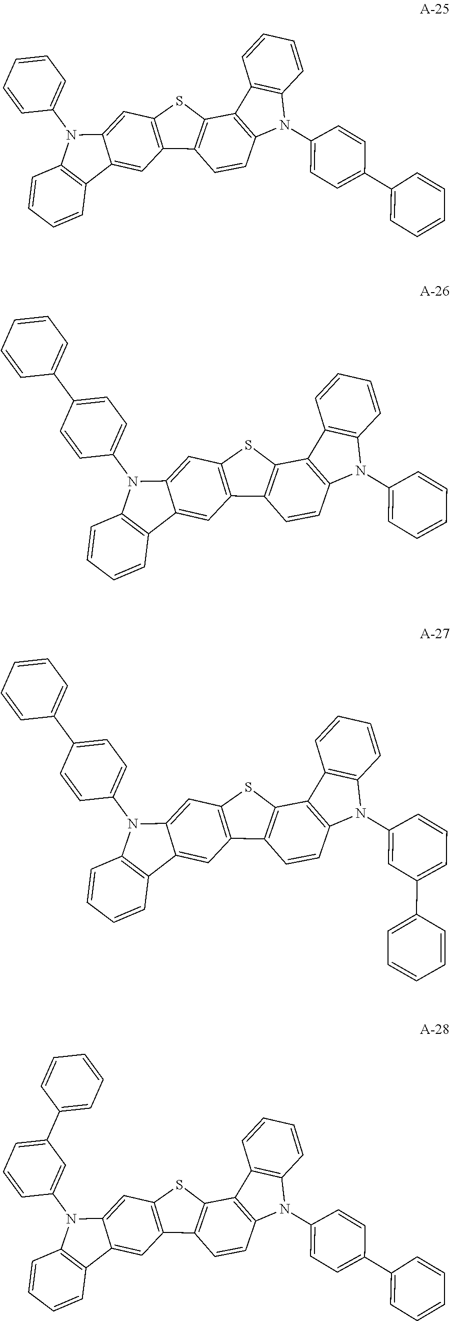 [SMILES: c1ccc(-c2ccc(-n3c4ccccc4c4c5sc6cc7c(cc6c5ccc43)c3ccccc3n7-c3ccccc3)cc2)cc1 ; c1ccc(-c2ccc(-n3c4ccccc4c4cc5c(cc43)sc3c5ccc4c3c3ccccc3n4-c3ccccc3)cc2)cc1 ; c1ccc(-c2ccc(-n3c4ccccc4c4cc5c(cc43)sc3c5ccc4c3c3ccccc3n4-c3cccc(-c4ccccc4)c3)cc2)cc1 ; c1ccc(-c2ccc(-n3c4ccccc4c4c5sc6cc7c(cc6c5ccc43)c3ccccc3n7-c3cccc(-c4ccccc4)c3)cc2)cc1]

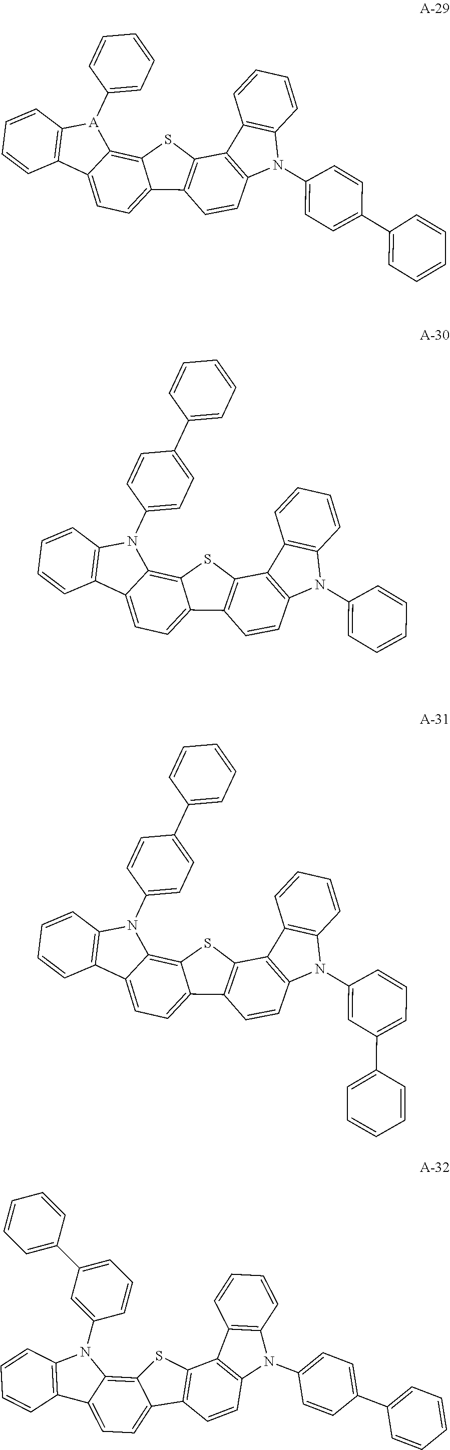

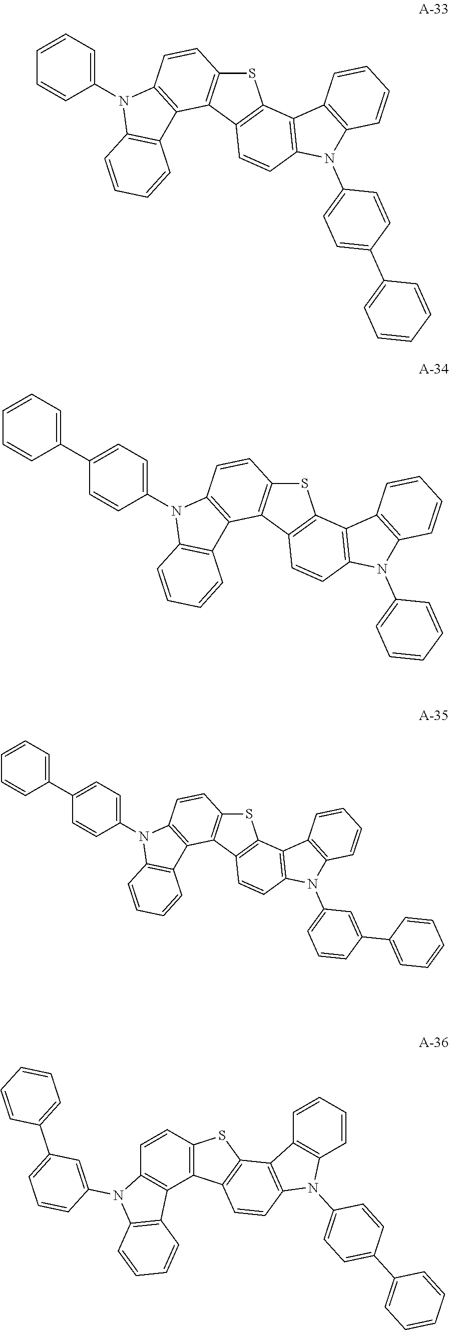 [SMILES: c1ccc(-c2ccc(-n3c4ccccc4c4c5sc6ccc7c(c8ccccc8n7-c7ccccc7)c6c5ccc43)cc2)cc1 ; c1ccc(-c2ccc(-n3c4ccccc4c4c5c(ccc43)sc3c5ccc4c3c3ccccc3n4-c3ccccc3)cc2)cc1 ; c1ccc(-c2ccc(-n3c4ccccc4c4c5c(ccc43)sc3c5ccc4c3c3ccccc3n4-c3cccc(-c4ccccc4)c3)cc2)cc1 ; c1ccc(-c2ccc(-n3c4ccccc4c4c5sc6ccc7c(c8ccccc8n7-c7cccc(-c8ccccc8)c7)c6c5ccc43)cc2)cc1]

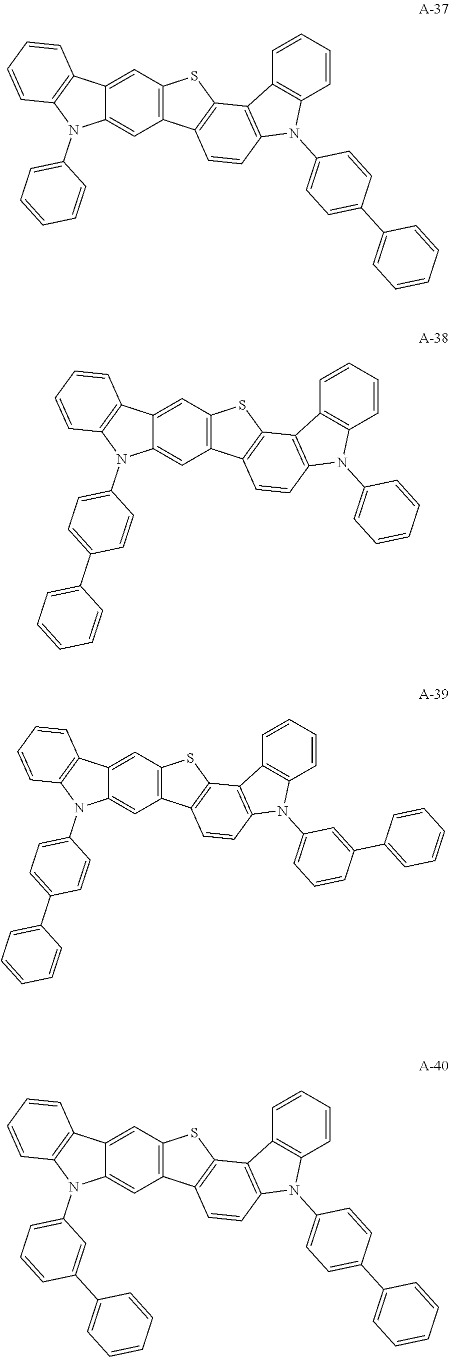 [SMILES: c1ccc(-c2ccc(-n3c4ccccc4c4c5sc6cc7c8ccccc8n(-c8ccccc8)c7cc6c5ccc43)cc2)cc1 ; c1ccc(-c2ccc(-n3c4ccccc4c4cc5sc6c(ccc7c6c6ccccc6n7-c6ccccc6)c5cc43)cc2)cc1 ; c1ccc(-c2ccc(-n3c4ccccc4c4cc5sc6c(ccc7c6c6ccccc6n7-c6cccc(-c7ccccc7)c6)c5cc43)cc2)cc1 ; c1ccc(-c2ccc(-n3c4ccccc4c4c5sc6cc7c8ccccc8n(-c8cccc(-c9ccccc9)c8)c7cc6c5ccc43)cc2)cc1]

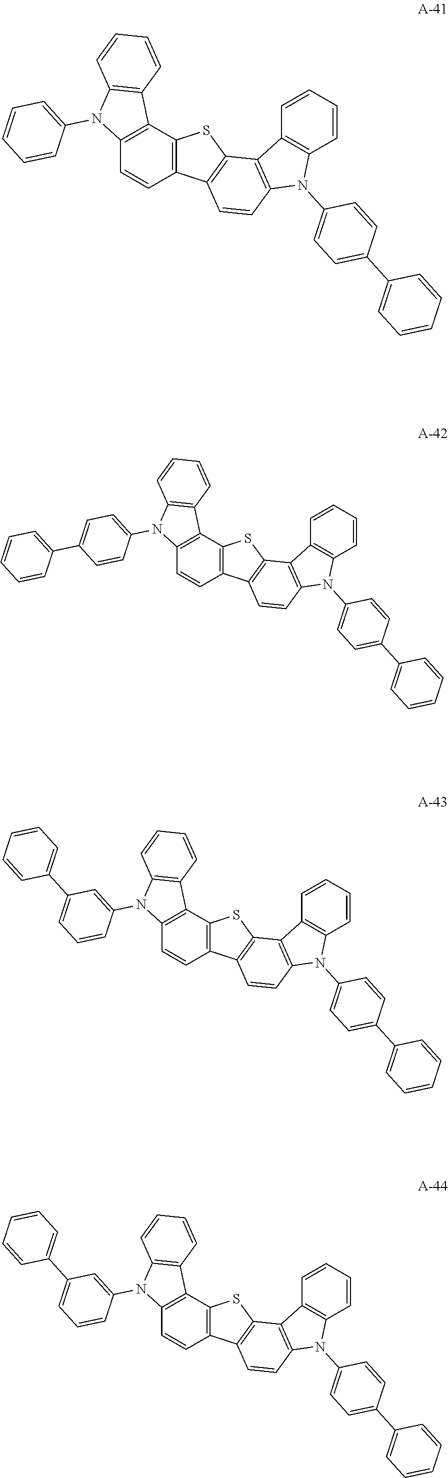 [SMILES: c1ccc(-c2ccc(-n3c4ccccc4c4c5sc6c(ccc7c6c6ccccc6n7-c6ccccc6)c5ccc43)cc2)cc1 ; c1ccc(-c2ccc(-n3c4ccccc4c4c5sc6c(ccc7c6c6ccccc6n7-c6ccc(-c7ccccc7)cc6)c5ccc43)cc2)cc1 ; c1ccc(-c2ccc(-n3c4ccccc4c4c5sc6c(ccc7c6c6ccccc6n7-c6cccc(-c7ccccc7)c6)c5ccc43)cc2)cc1 ; c1ccc(-c2ccc(-n3c4ccccc4c4c5sc6c(ccc7c6c6ccccc6n7-c6cccc(-c7ccccc7)c6)c5ccc43)cc2)cc1]

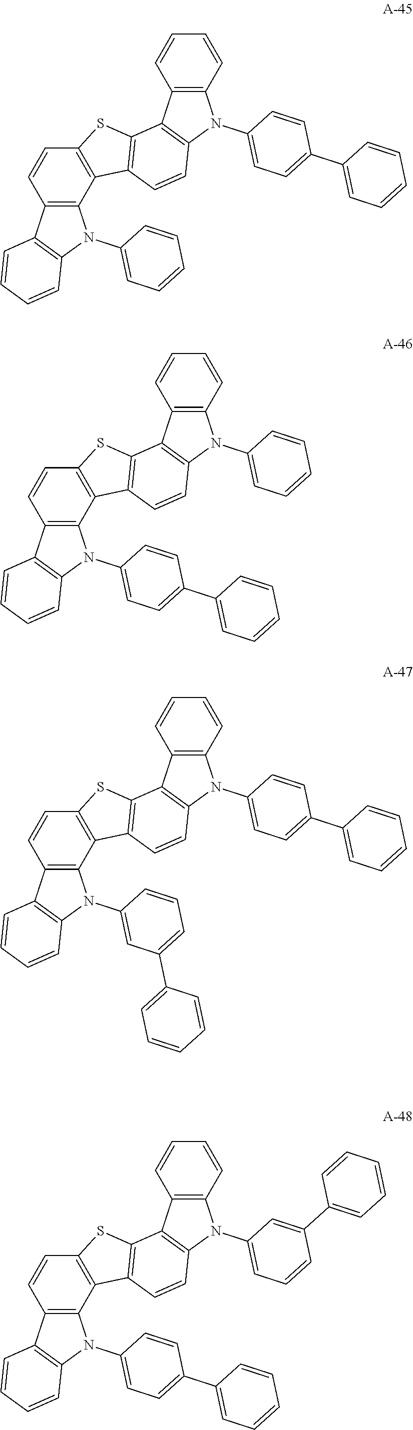 [SMILES: c1ccc(-c2ccc(-n3c4ccccc4c4c5sc6ccc7c8ccccc8n(-c8ccccc8)c7c6c5ccc43)cc2)cc1 ; c1ccc(-c2ccc(-n3c4ccccc4c4ccc5sc6c(ccc7c6c6ccccc6n7-c6ccccc6)c5c43)cc2)cc1 ; c1ccc(-c2ccc(-n3c4ccccc4c4c5sc6ccc7c8ccccc8n(-c8cccc(-c9ccccc9)c8)c7c6c5ccc43)cc2)cc1 ; c1ccc(-c2ccc(-n3c4ccccc4c4ccc5sc6c(ccc7c6c6ccccc6n7-c6cccc(-c7ccccc7)c6)c5c43)cc2)cc1]

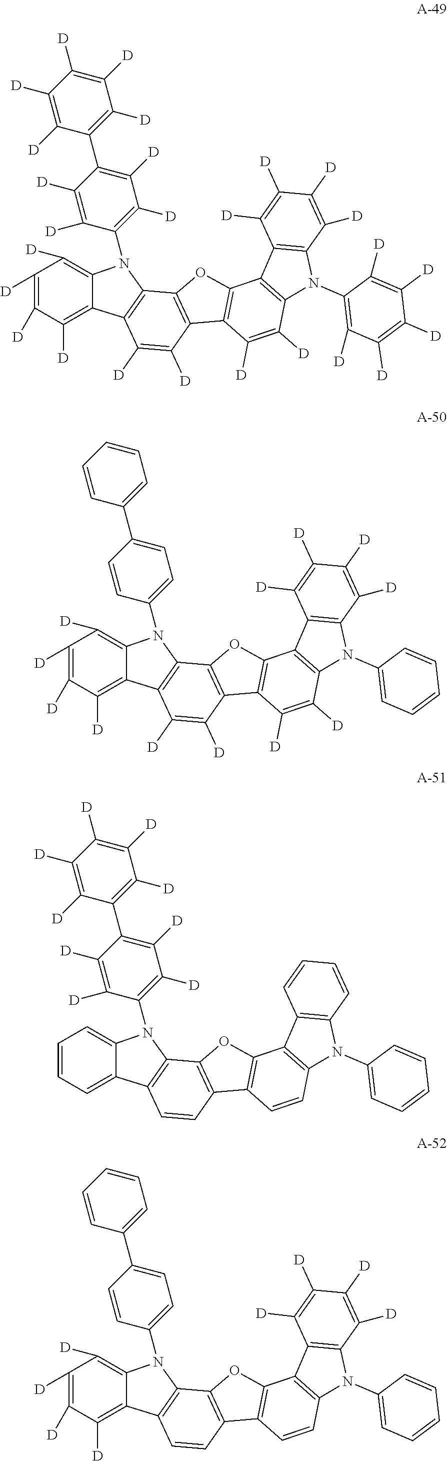

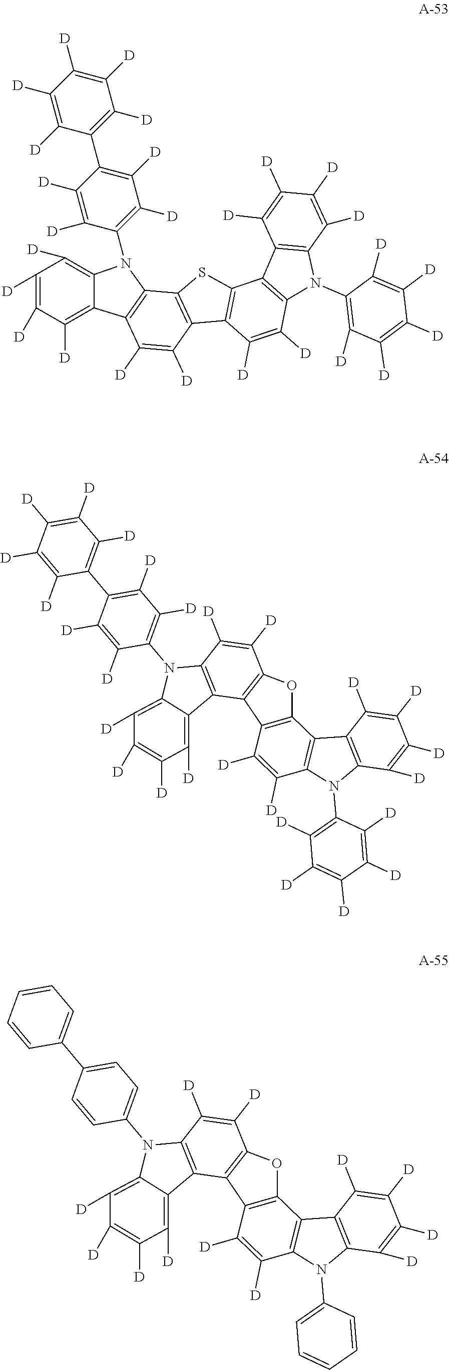

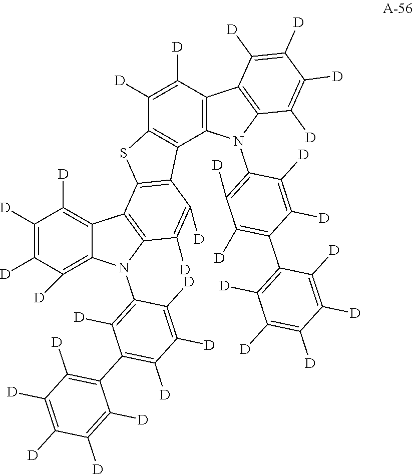 [SMILES: [2H]c1c([2H])c([2H])c(-c2c([2H])c([2H])c(-n3c4c([2H])c([2H])c([2H])c([2H])c4c4c([2H])c([2H])c5sc6c(c([2H])c([2H])c7c6c6c([2H])c([2H])c([2H])c([2H])c6n7-c6c([2H])c([2H])c([2H])c(-c7c([2H])c([2H])c([2H])c([2H])c7[2H])c6[2H])c5c43)c([2H])c2[2H])c([2H])c1[2H]]

In an embodiment, the second compound may be represented by Chemical Formula 3-I or Chemical Formula 3-II.

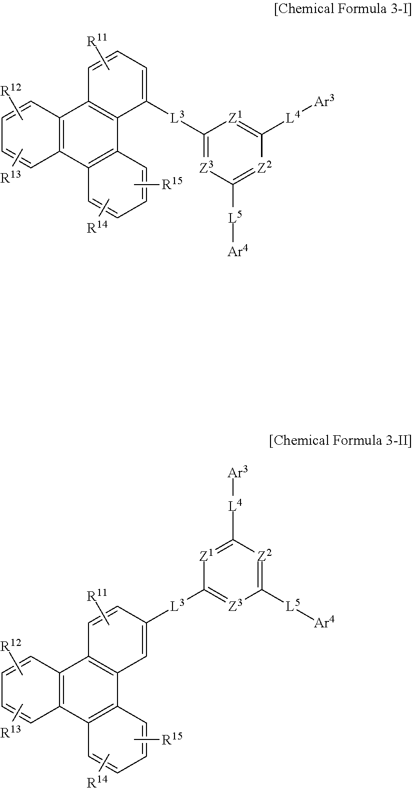

In Chemical Formula 3-I or Chemical Formula 3-II, Z¹ to Z³, L³ to L⁵, Ar³, Ar⁴, and R¹¹ to R¹⁵ are the same as described above.

In a specific embodiment, the second compound may be represented by Chemical Formula 3-II.

For example, each of Z¹ to Z³ may be N.

For example, Ar³ and Ar⁴ may each independently be a substituted or unsubstituted phenyl group, a substituted or unsubstituted biphenyl group, or a substituted or unsubstituted naphthyl group.

For example, Ar³ and Ar⁴ may each independently be selected from the substituents listed in Group II.

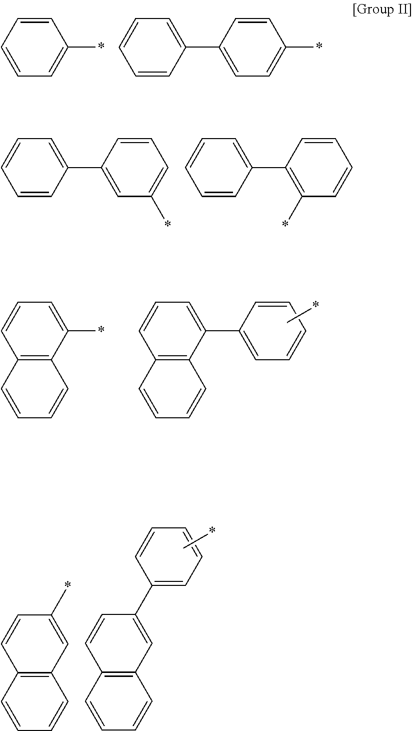

In Group II, * is a linking point.

For example, L³ to L⁵ may each independently be a single bond or a substituted or unsubstituted phenylene group.

As a specific example, L³ may be a substituted or unsubstituted phenylene group or a substituted or unsubstituted biphenylene group, and L⁴ and L⁵ may each independently be a single bond or a substituted or unsubstituted phenylene group.

For example, L³ may be a substituted or unsubstituted meta-phenylene group or a substituted or unsubstituted meta-biphenylene group.

For example, R¹¹ to R¹⁵ may each independently be hydrogen, deuterium, a cyano group, a halogen group, a substituted or unsubstituted C1 to C10 alkyl group, or a substituted or unsubstituted C6 to C12 aryl group.

As a specific example, R¹¹ to R¹⁵ may each independently be hydrogen, deuterium, a substituted or unsubstituted C1 to C5 alkyl group, a substituted or unsubstituted phenyl group, a substituted or unsubstituted biphenyl group, or a substituted or unsubstituted naphthyl group.

For example, each of R¹¹ to R¹⁵ may be hydrogen.

For example, the second compound may be one selected from compounds listed in Group 2.

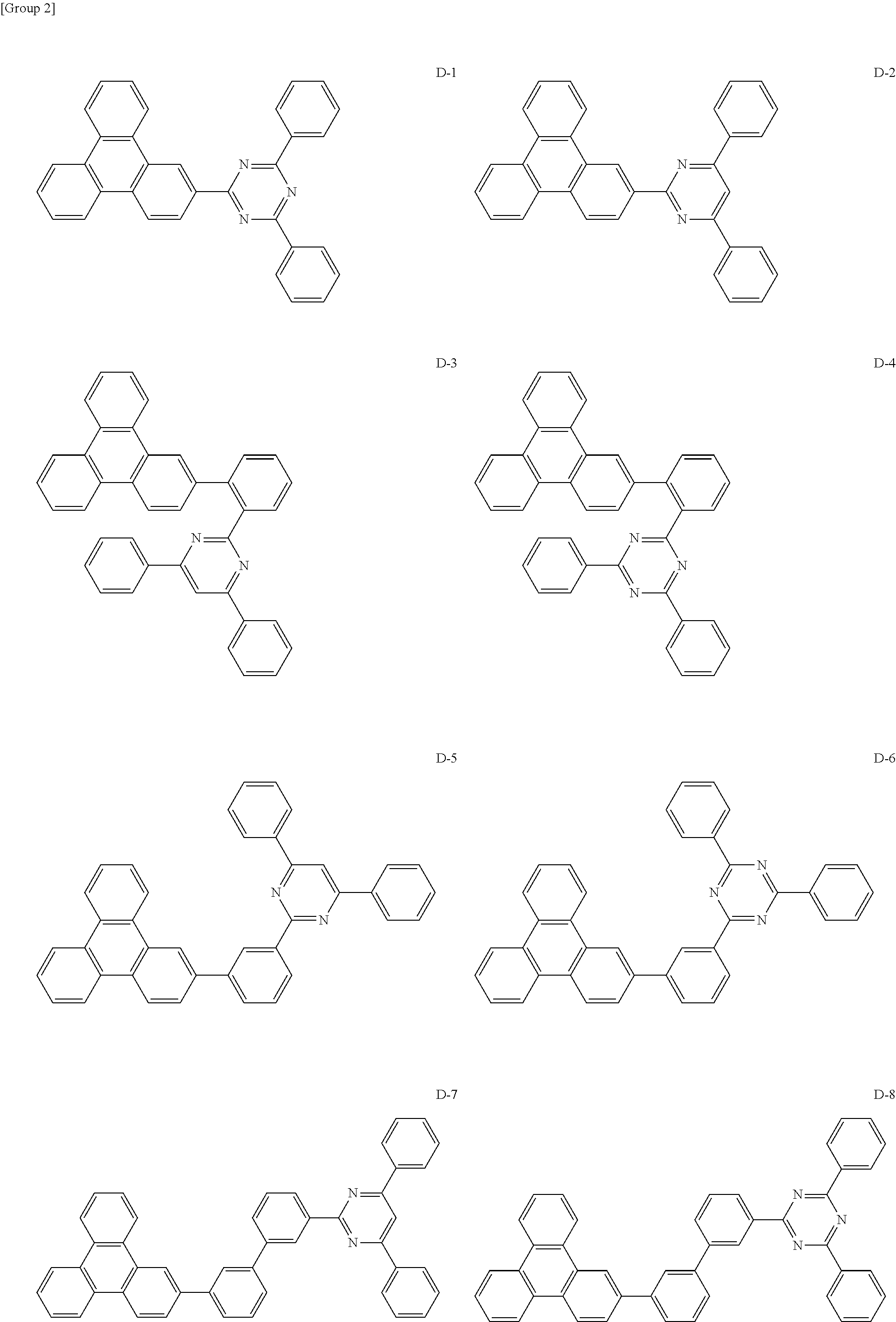

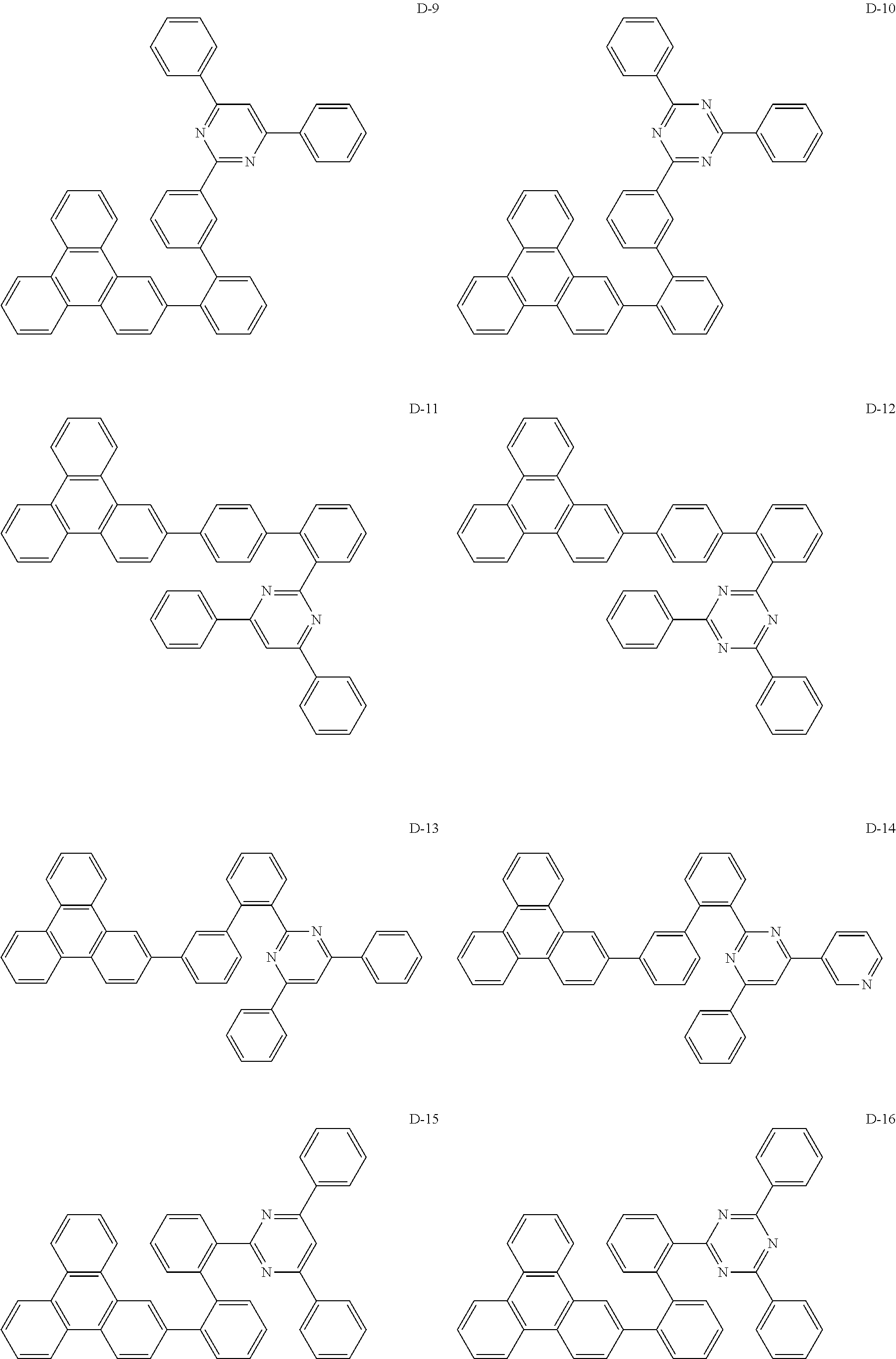

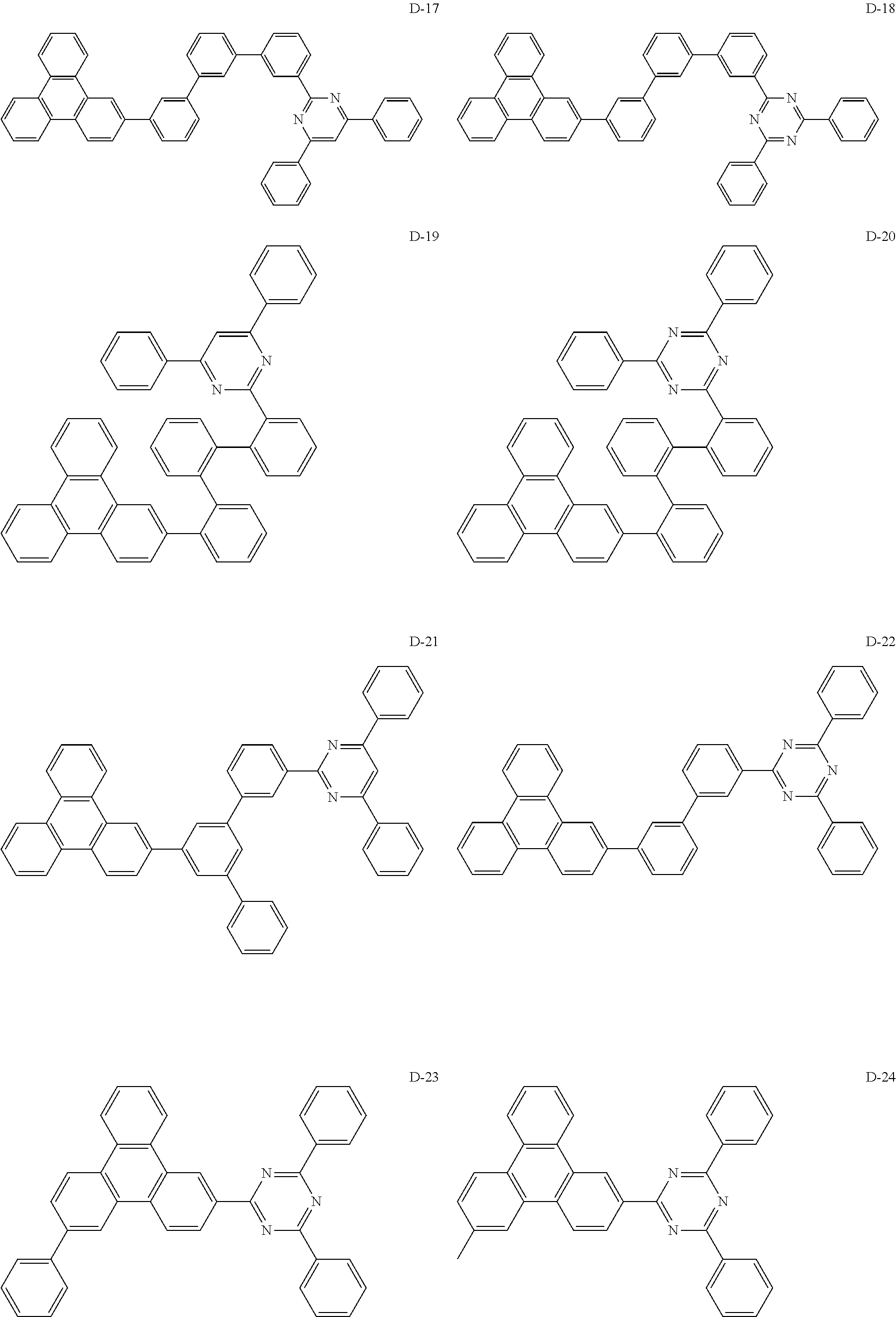

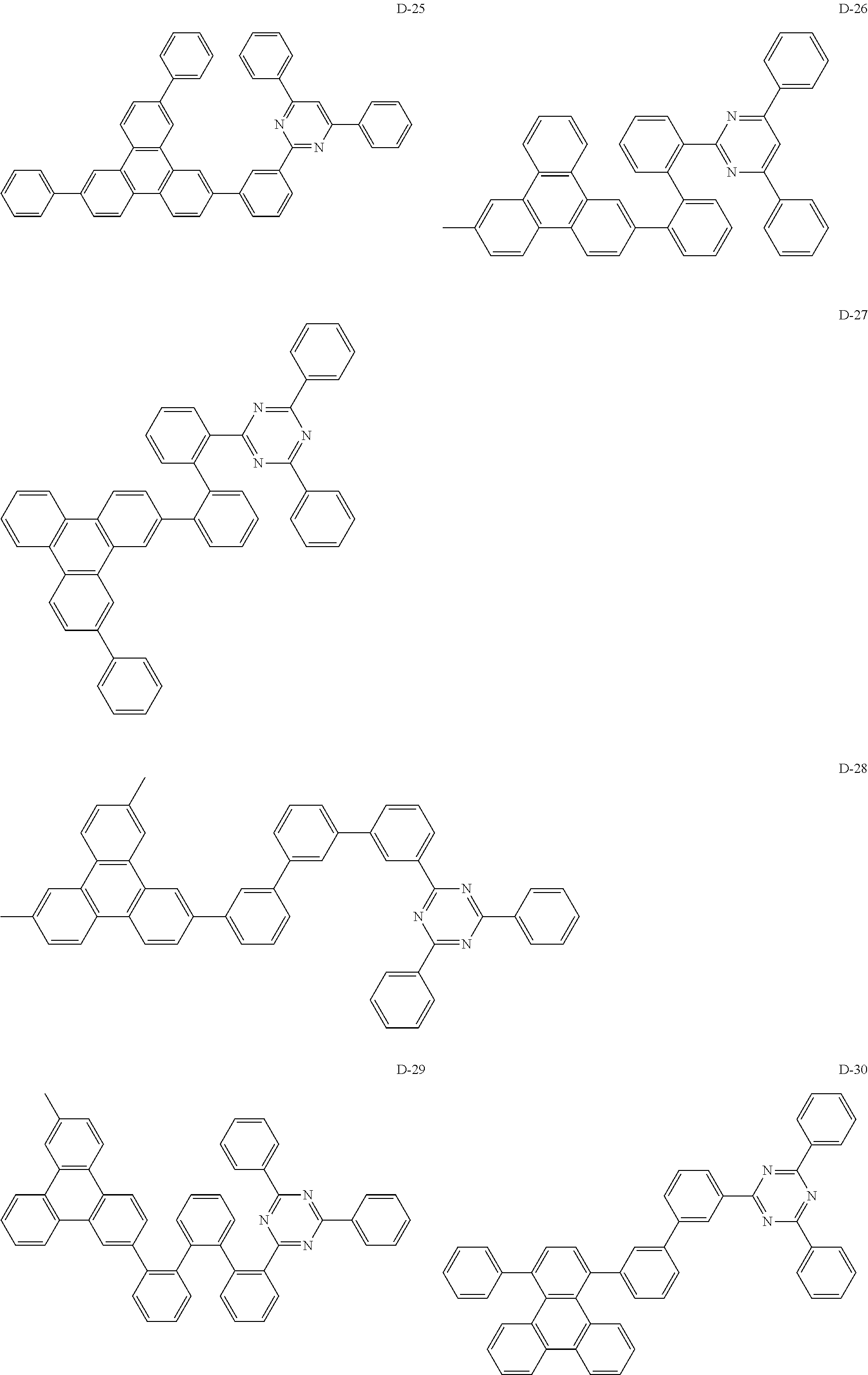

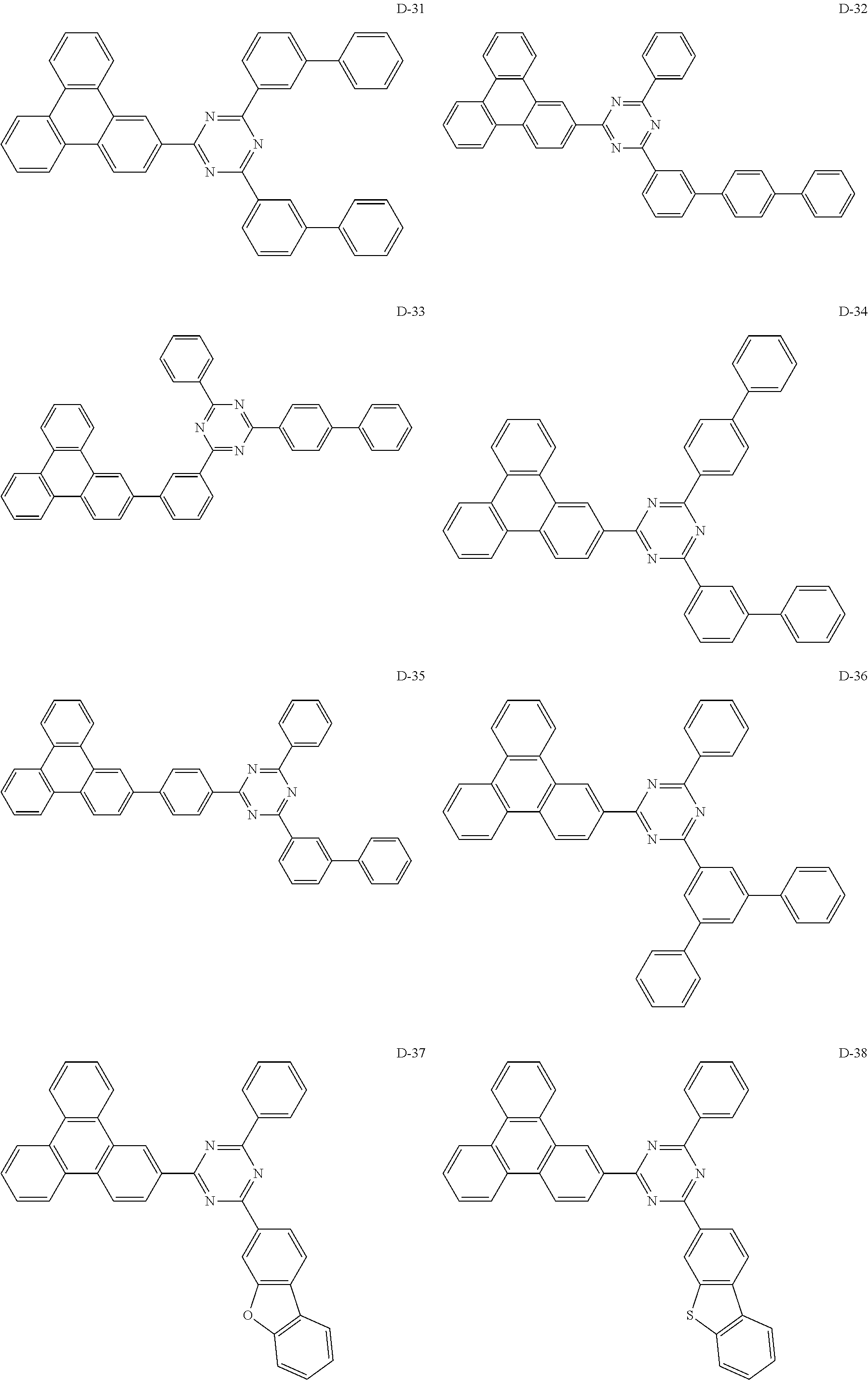

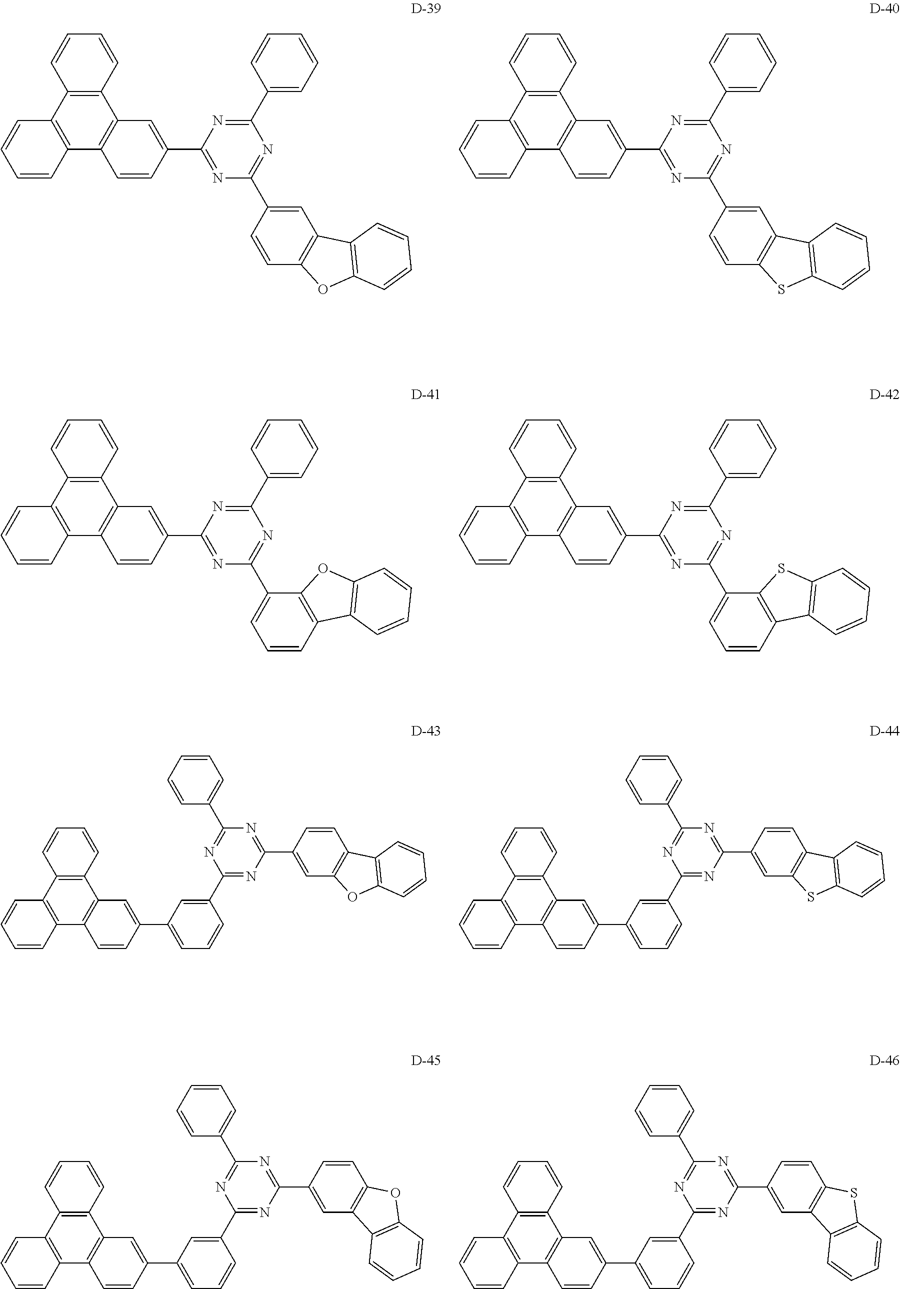

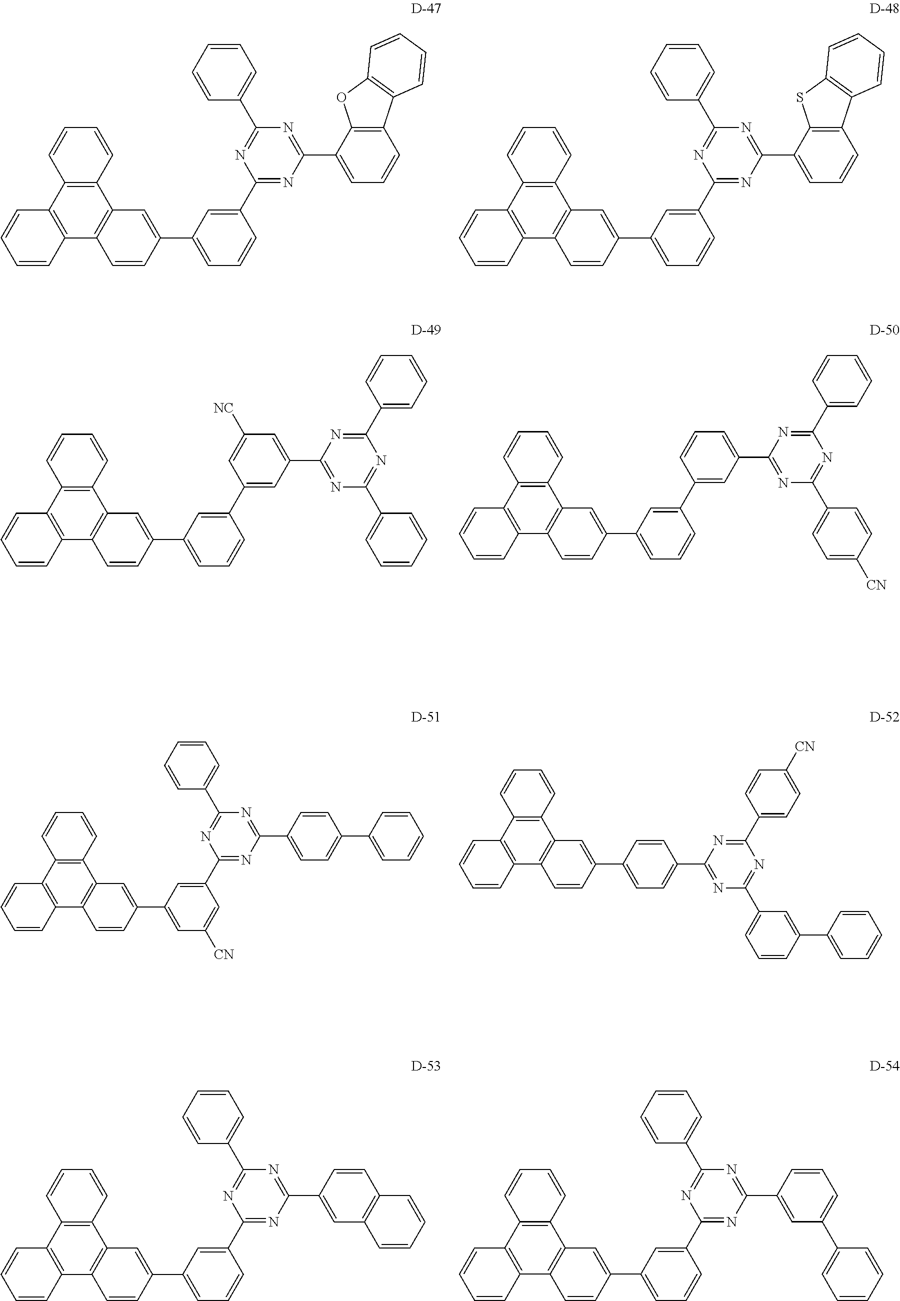

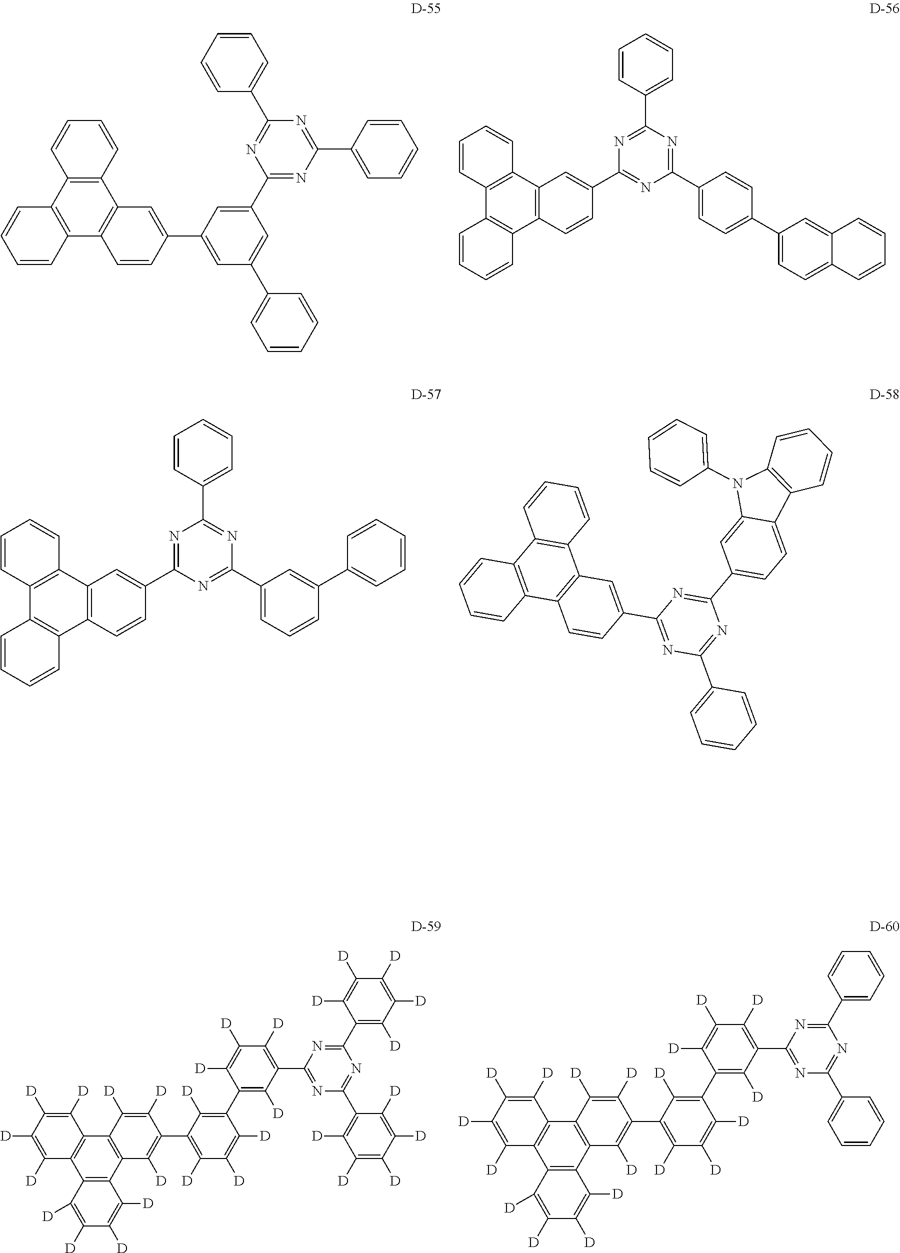

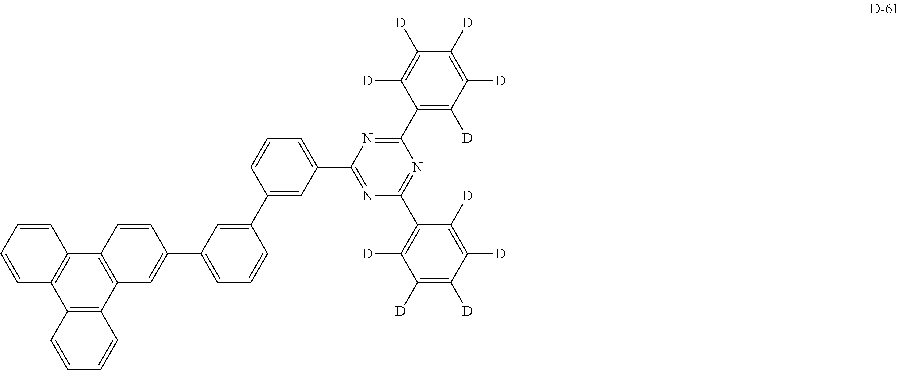 [SMILES: [2H]c1c([2H])c([2H])c(-c2nc(-c3cccc(-c4cccc(-c5ccc6c7ccccc7c7ccccc7c6c5)c4)c3)nc(-c3c([2H])c([2H])c([2H])c([2H])c3[2H])n2)c([2H])c1[2H]]

The composition for an organic optoelectronic device according to a more specific embodiment of the present invention may include a first compound represented by Chemical Formula 1A or Chemical Formula 1B and a second compound represented by Chemical Formula 3-II.

The first compound and the second compound may be for example included in a weight ratio of 1:99 to 99:1. Within the range, a desirable weight ratio may be adjusted using a hole transport capability of the first compound and an electron transport capability of the second compound to realize bipolar characteristics and thus to improve efficiency and life-span. Within the range, they may be for example included in a weight ratio of about 90:10 to 10:90, about 80:20 to 10:90, about 70:30 to 10:90, or about 60:40 to 10:90. For example, they may be included in a weight ratio of 60:40 to 20:80, for example, 60:40 to 30:70.

According to the most specific embodiment, they may be included in a weight ratio of about 50:50 to about 30:70.

In an embodiment of the present invention, each of the first compound and the second compound may be included as a host of the light emitting layer, for example, a phosphorescent host.

The aforementioned composition for an organic optoelectronic device may be formed into a film by a dry film formation method such as chemical vapor deposition (CVD).

Hereinafter, an organic optoelectronic device including the aforementioned composition for an organic optoelectronic device is described.

The organic optoelectronic device may be any device to convert electrical energy into photoenergy and vice versa without particular limitation, and may be, for example an organic photoelectric device, an organic light emitting diode, an organic solar cell, and an organic photoconductor drum.

Herein, an organic light emitting diode as one example of an organic optoelectronic device is described referring to drawings.

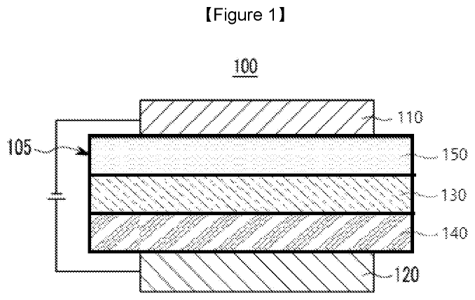

FIG. 1 is a cross-sectional view illustrating an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, LiF/Al, and BaF₂/Ca, but is not limited thereto.

The organic layer 105 may include the aforementioned composition for an organic optoelectronic device.

The organic layer 105 may include the light emitting layer 130, and the light emitting layer 130 may include the aforementioned composition for an organic optoelectronic device.

The light emitting layer 130 may include, for example, the aforementioned composition for an organic optoelectronic device as a phosphorescent host.

The light emitting layer may further include one or more compounds in addition to the aforementioned host.

The light emitting layer may further include a dopant. The dopant may be, for example, a phosphorescent dopant, such as a phosphorescent dopant of red, green, or blue color, and may be, for example, a red or green phosphorescent dopant.

The composition for an organic optoelectronic device further including a dopant may be, for example, a red or green light emitting composition.

The dopant is a material mixed with the compound or composition for an organic optoelectronic device in a small amount to cause light emission, and may be generally a material such as a metal complex that emits light by multiple excitation into a triplet or more. The dopant may be, for example, an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z, but is not limited thereto.

L⁶MX² [Chemical Formula Z]

In Chemical Formula Z, M is a metal, and L⁶ and X² are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof and L⁶ and X² may be, for example a bidentate ligand.

The organic layer may further include a charge transport region in addition to the light emitting layer.

The charge transport region may be, for example, a hole transport region 140.

The hole transport region 140 may further increase hole injection and/or hole mobility and block electrons between the anode 120 and the light emitting layer 130.

Specifically, the hole transport region 140 may include a hole transport layer between the anode 120 and the light emitting layer 130, and a hole transport auxiliary layer between the light emitting layer 130 and the hole transport layer, and at least one of the compounds of Group A may be included in at least one of the hole transport layer and the hole transport auxiliary layer.

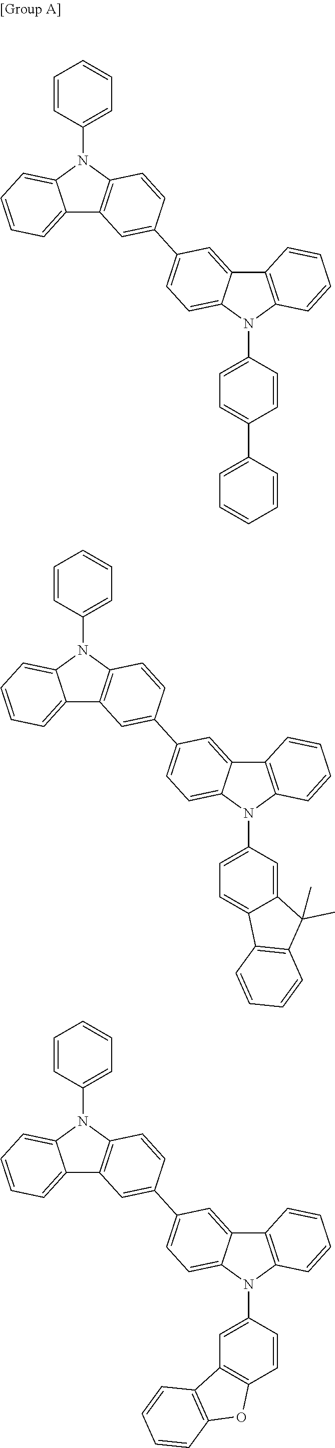 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

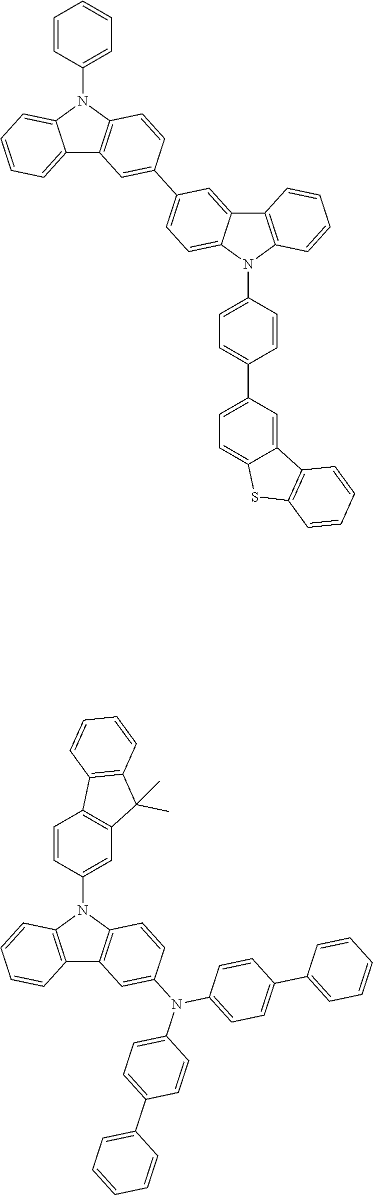 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

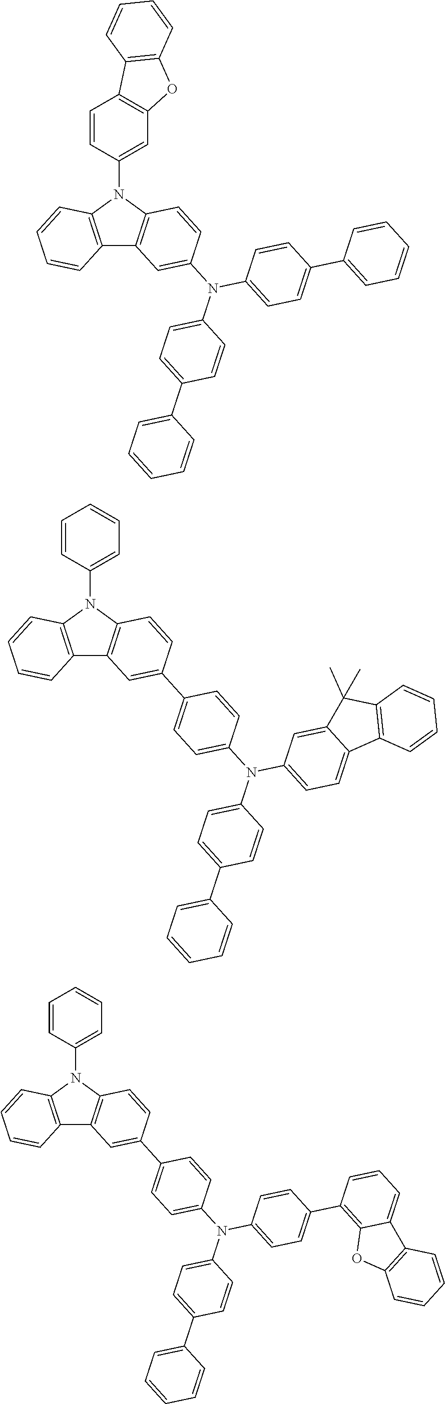 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

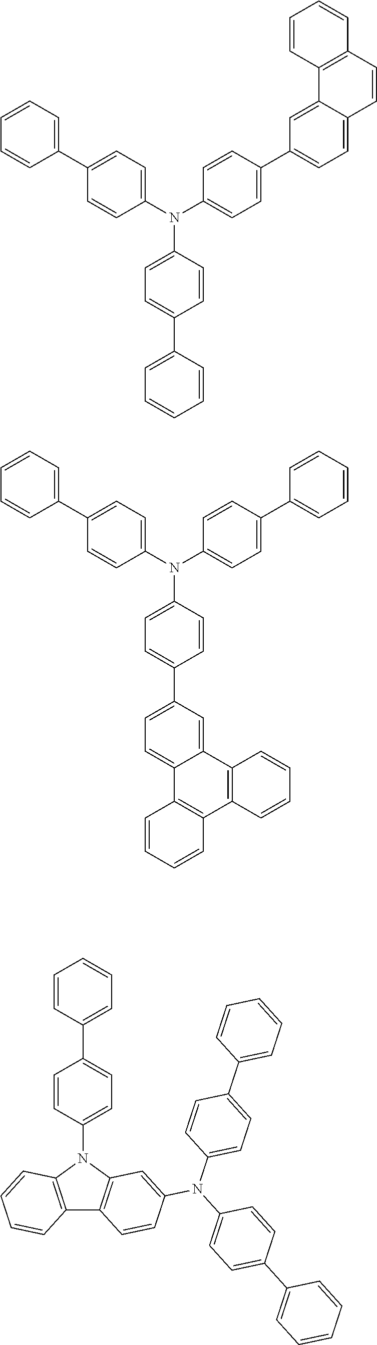 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

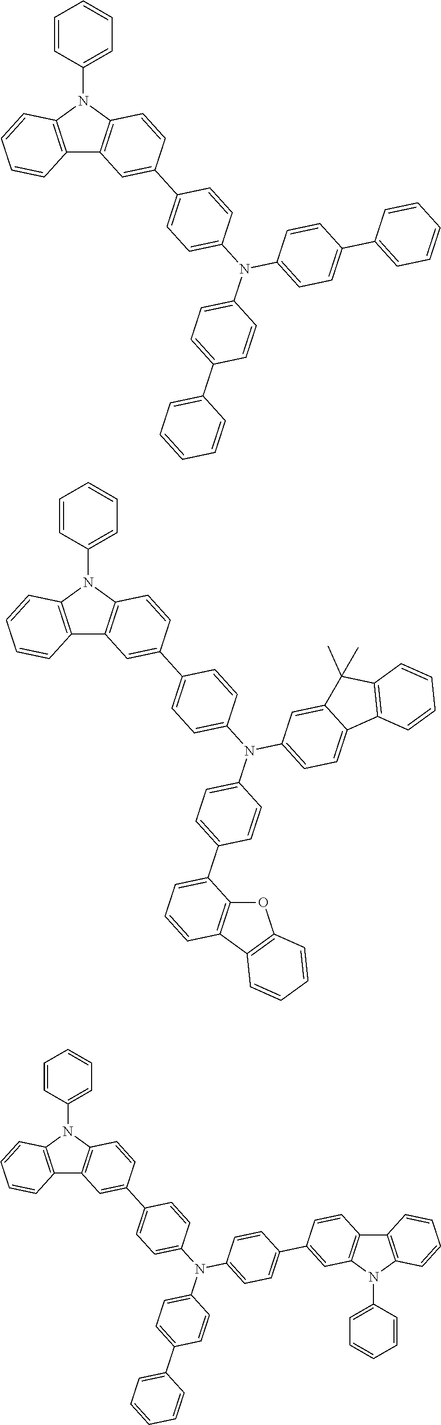 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

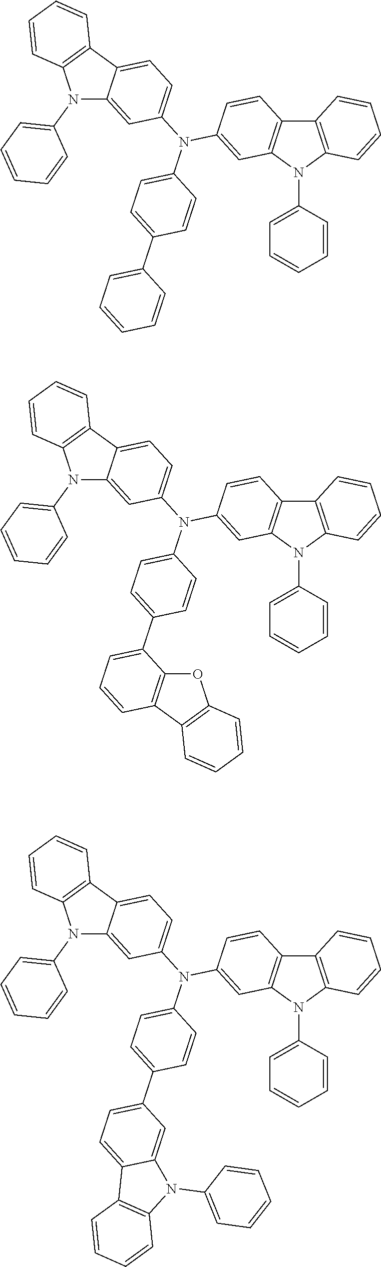 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

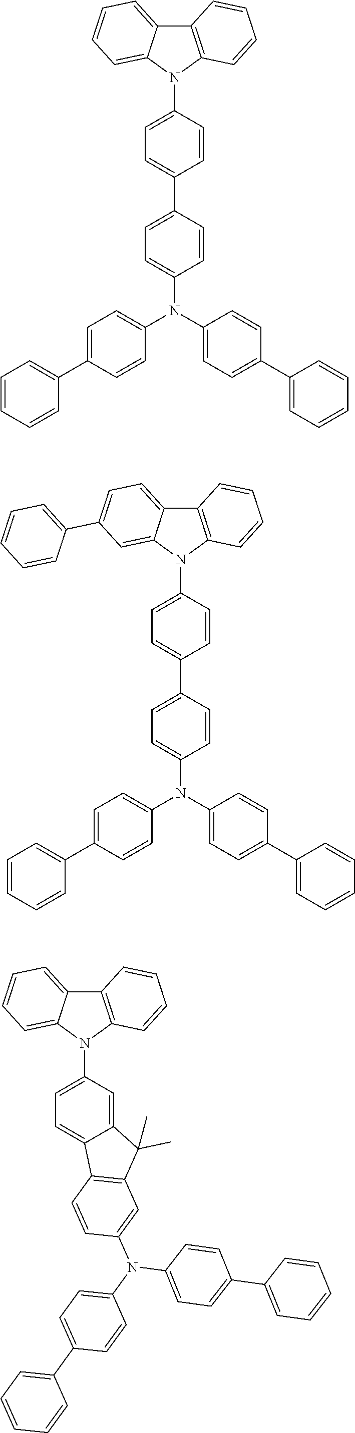 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21]

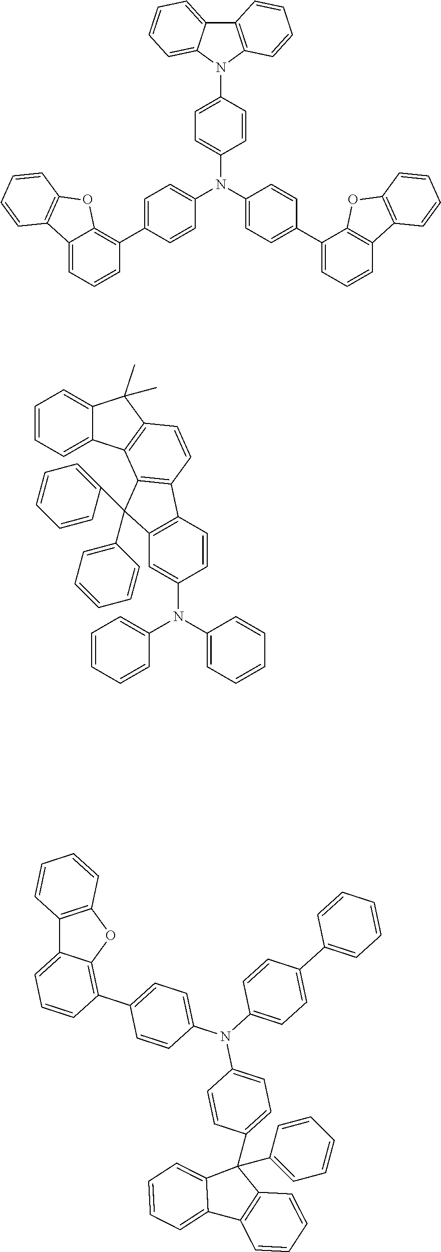 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1]

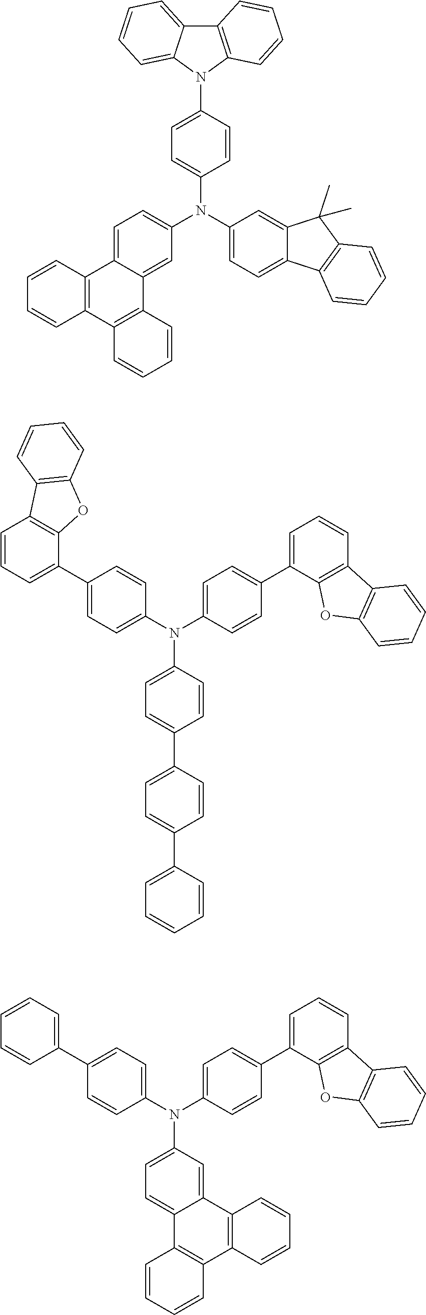 [SMILES: CC1(C)c2ccccc2-c2ccc(N(c3ccc(-n4c5ccccc5c5ccccc54)cc3)c3ccc4c5ccccc5c5ccccc5c4c3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1]

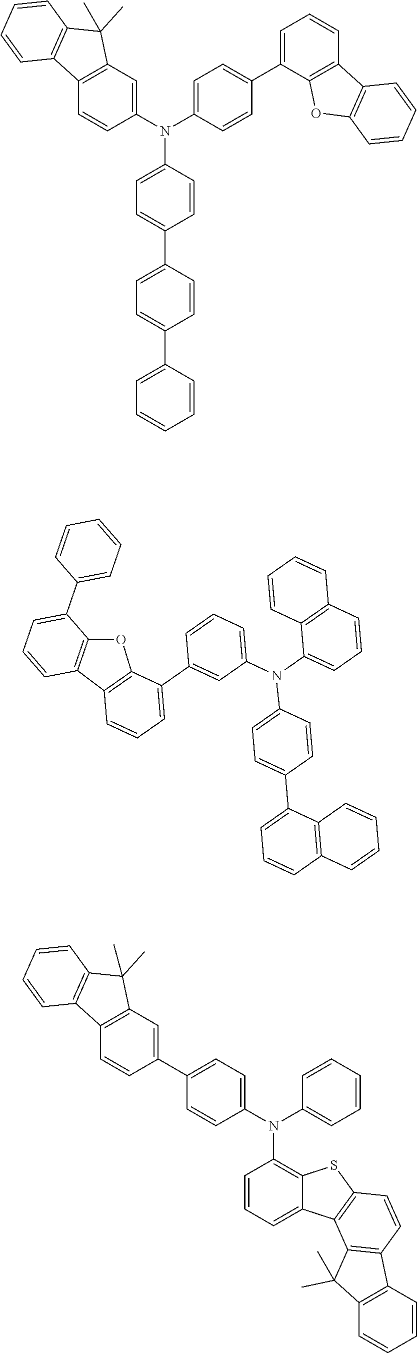 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21]

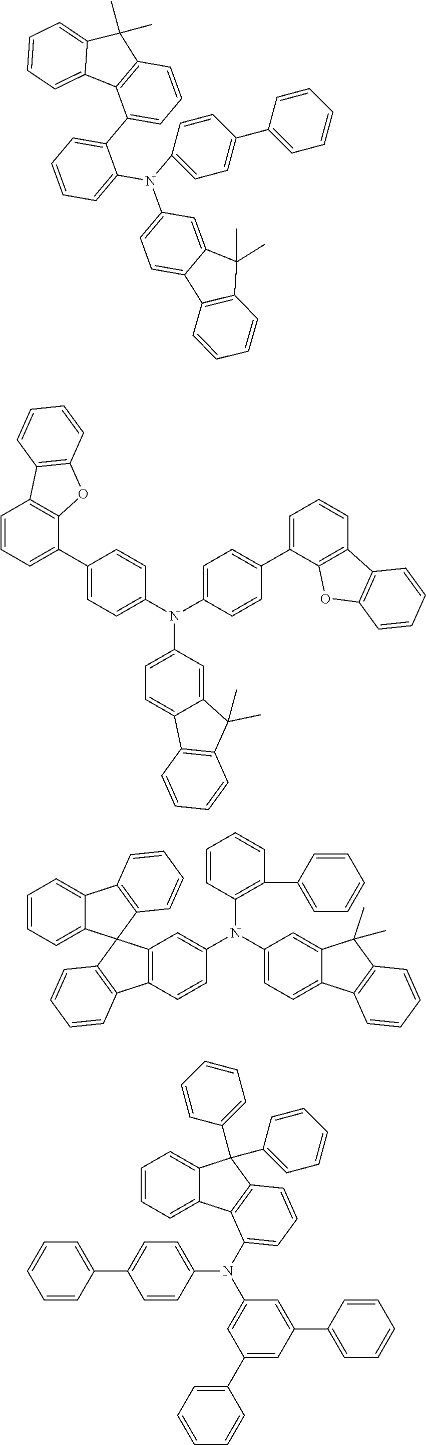

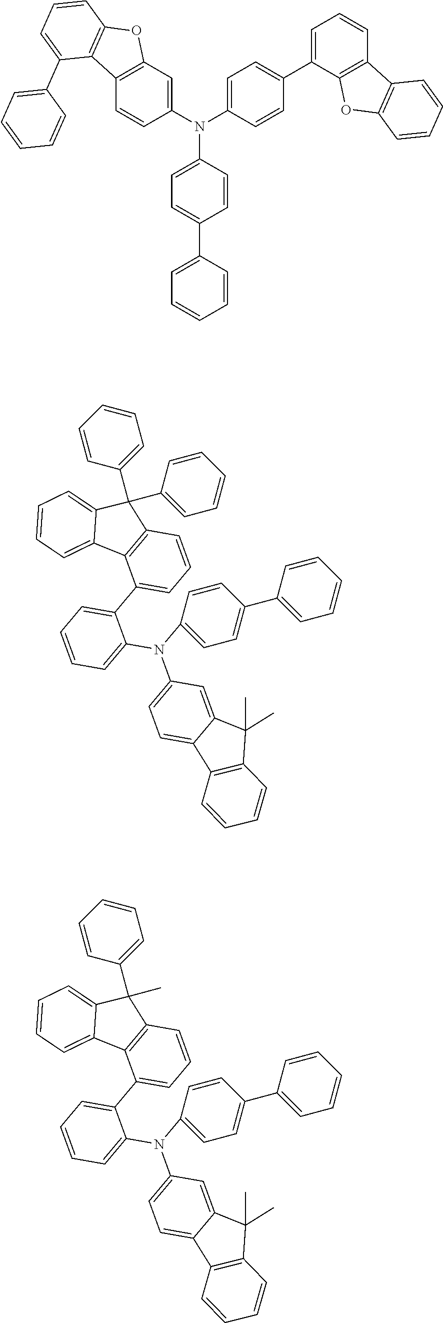 [SMILES: c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21]

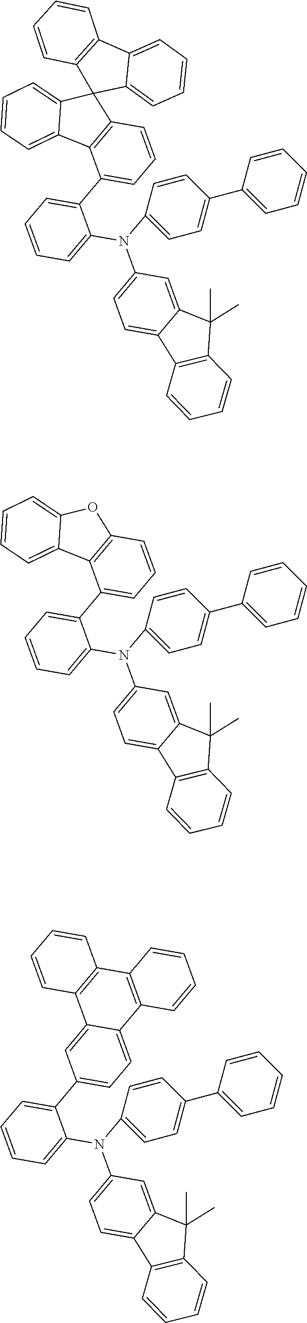 [SMILES: CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21 ; CC1(C)c2ccccc2-c2ccc(N(c3ccc(-c4ccccc4)cc3)c3ccccc3-c3ccc4c5ccccc5c5ccccc5c4c3)cc21]

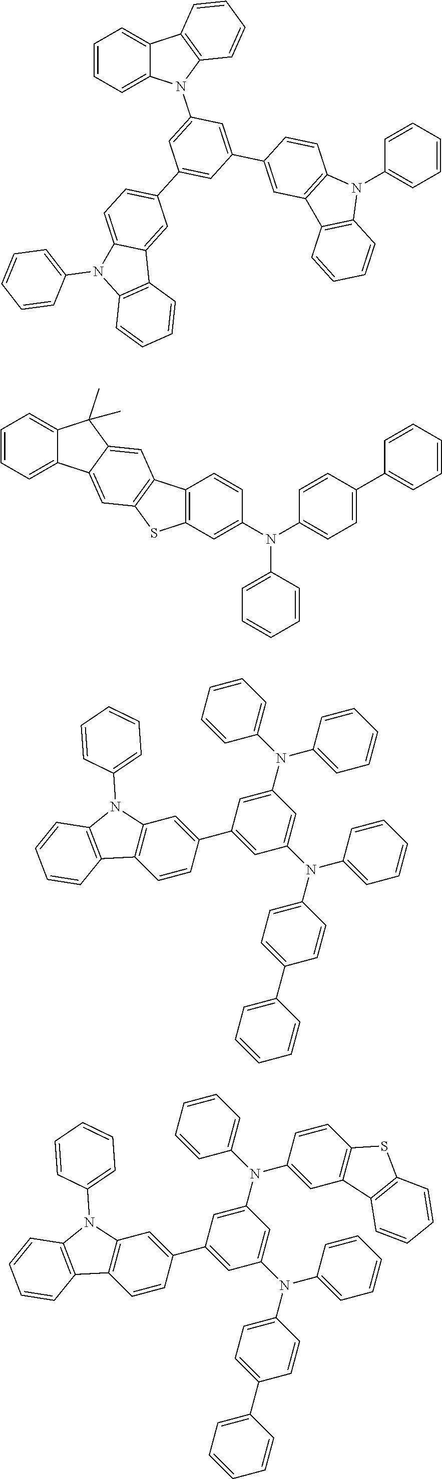 [SMILES: c1ccc(-n2c3ccccc3c3cc(-c4cc(-c5ccc6c(c5)c5ccccc5n6-c5ccccc5)cc(-n5c6ccccc6c6ccccc65)c4)ccc32)cc1 ; CC1(C)c2ccccc2-c2cc3sc4cc(N(c5ccccc5)c5ccc(-c6ccccc6)cc5)ccc4c3cc21 ; c1ccc(-c2ccc(N(c3ccccc3)c3cc(-c4ccc5c6ccccc6n(-c6ccccc6)c5c4)cc(N(c4ccccc4)c4ccccc4)c3)cc2)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1]

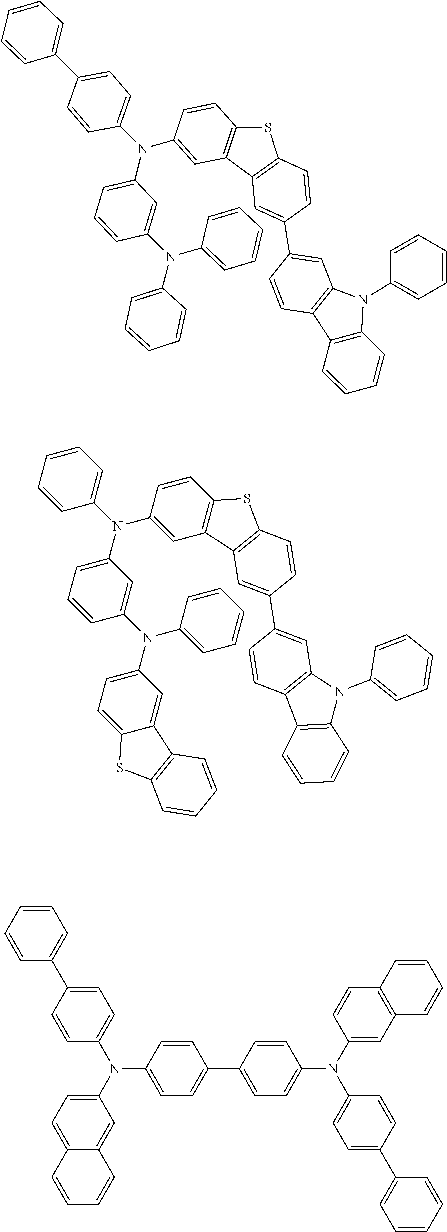 [SMILES: c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1]

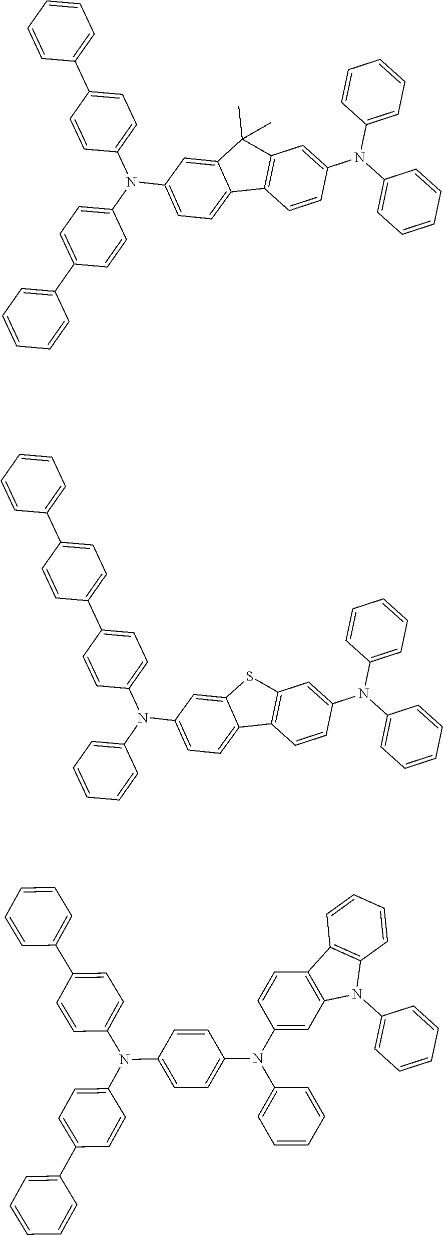 [SMILES: CC1(C)c2cc(N(c3ccccc3)c3ccccc3)ccc2-c2ccc(N(c3ccc(-c4ccccc4)cc3)c3ccc(-c4ccccc4)cc3)cc21 ; c1ccc(-c2ccc(-c3ccc(N(c4ccccc4)c4ccc5c(c4)sc4cc(N(c6ccccc6)c6ccccc6)ccc45)cc3)cc2)cc1 ; c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1]

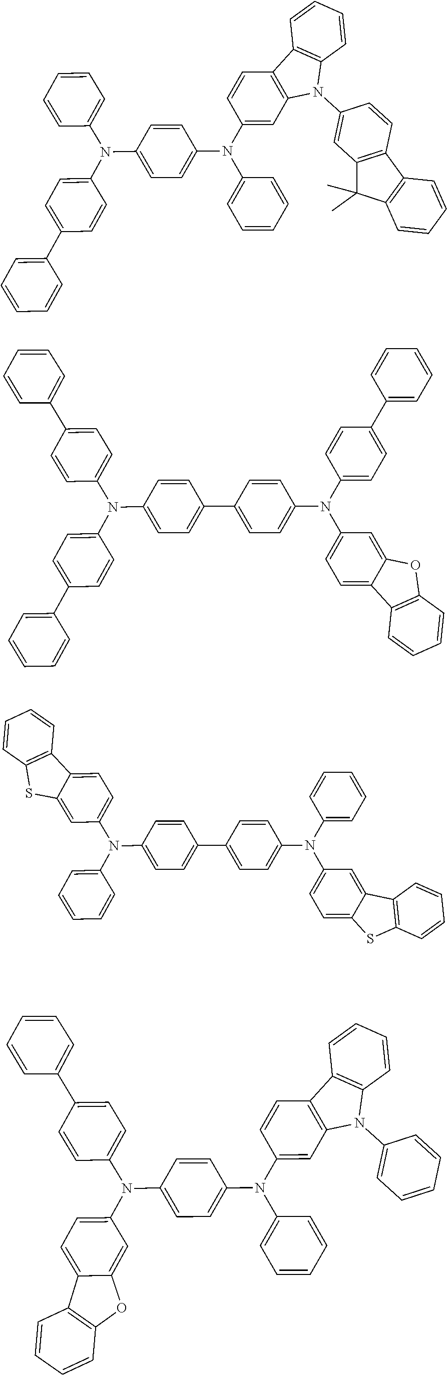

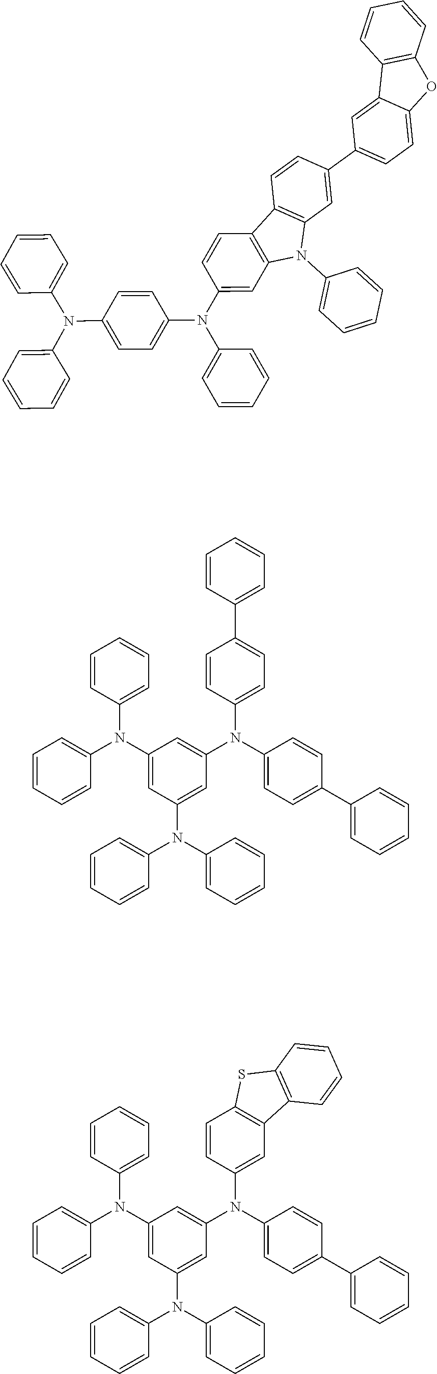 [SMILES: c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1]

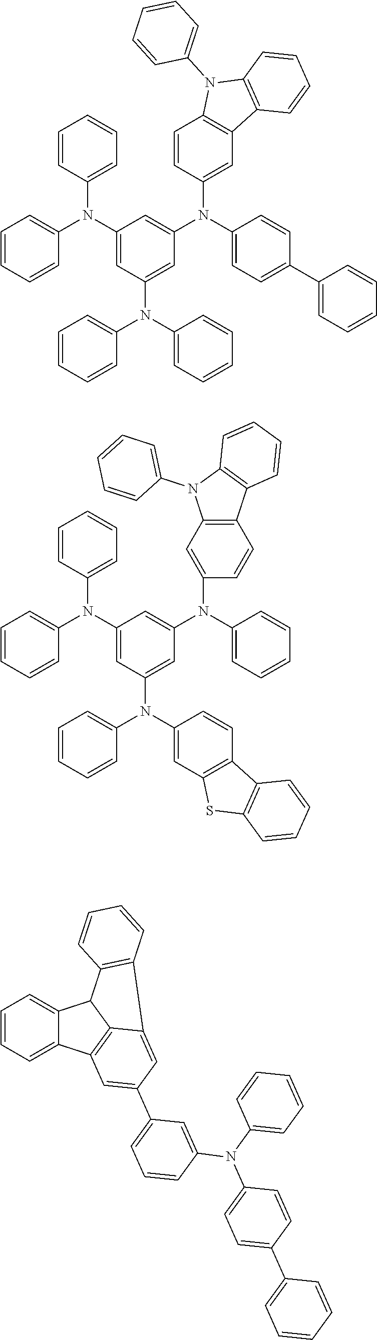 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6c(c4)-c4ccccc4C6c4ccccc4-5)c3)cc2)cc1]

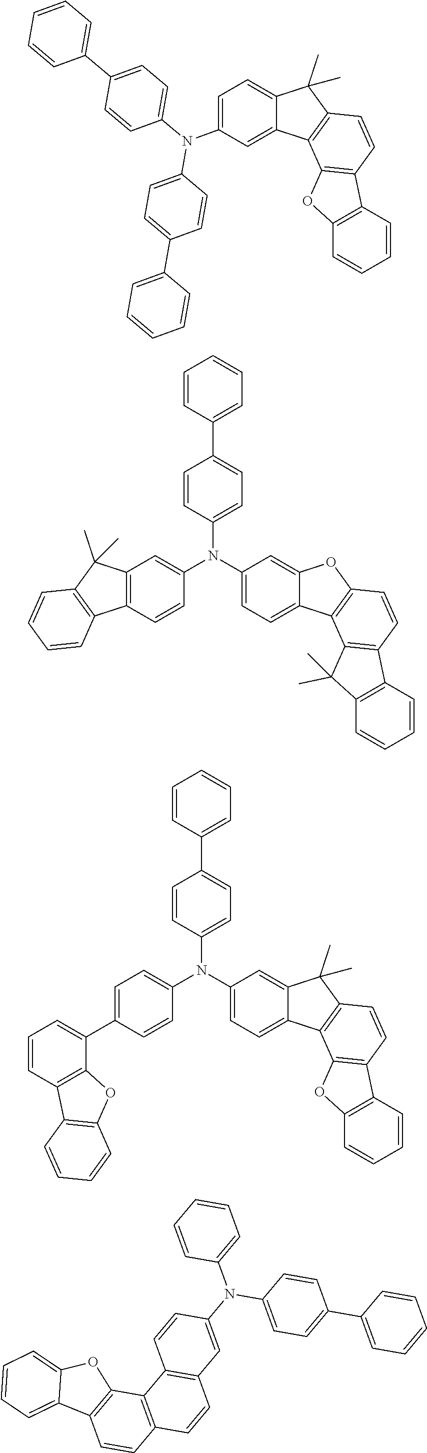

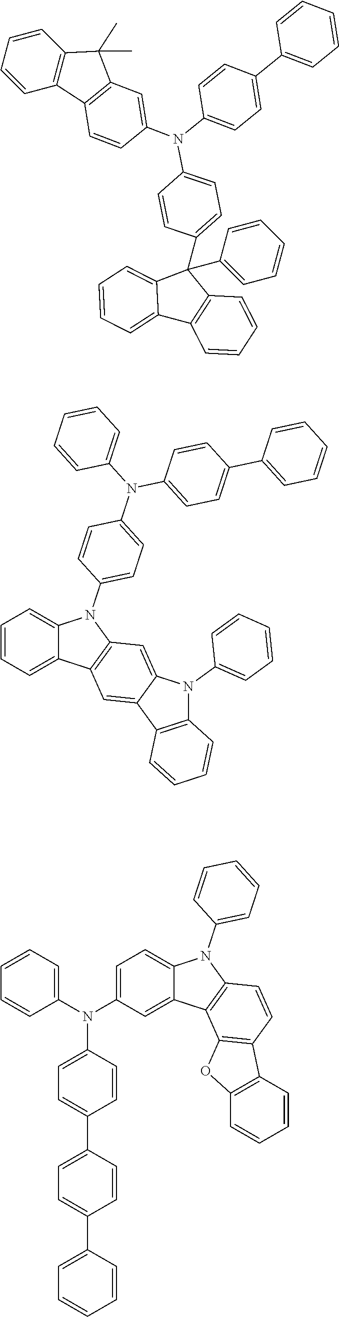 [SMILES: CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1]

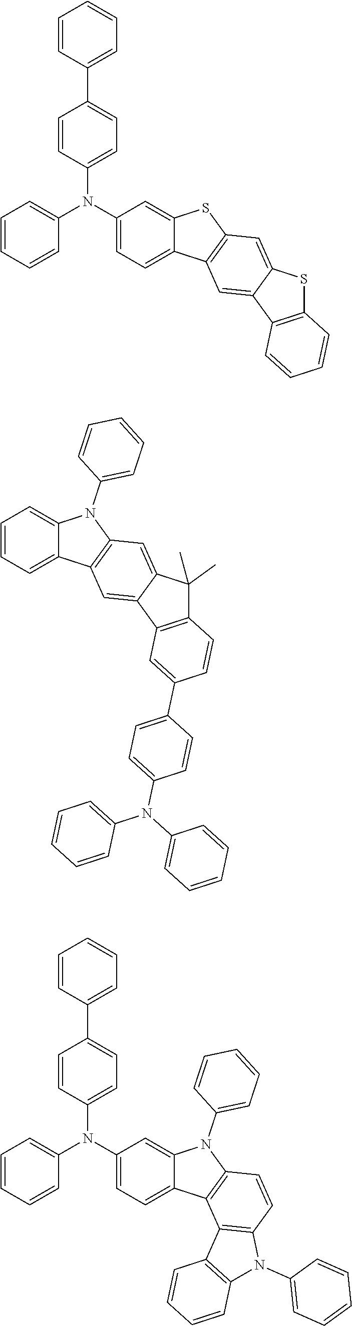 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

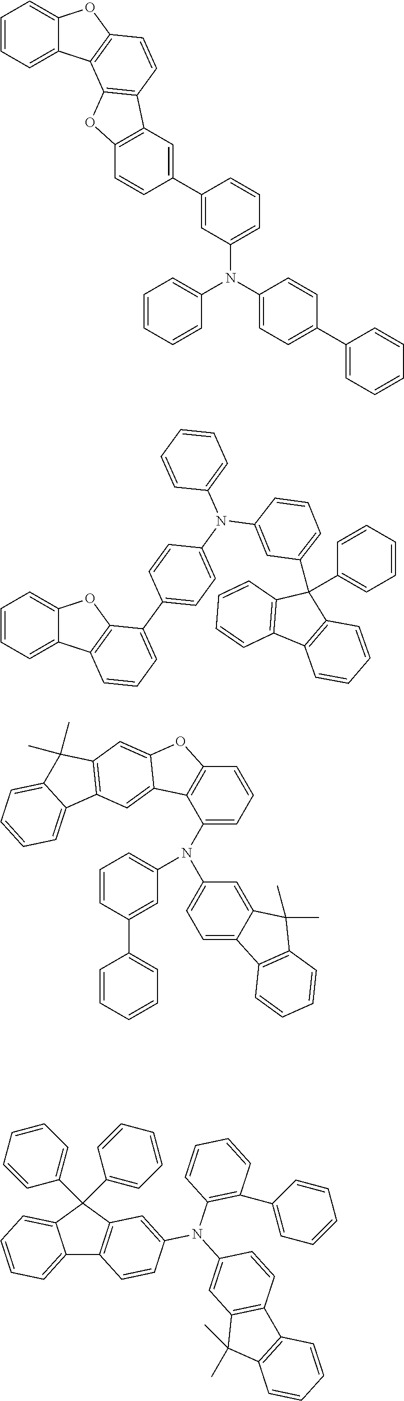 [SMILES: c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1 ; CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21]

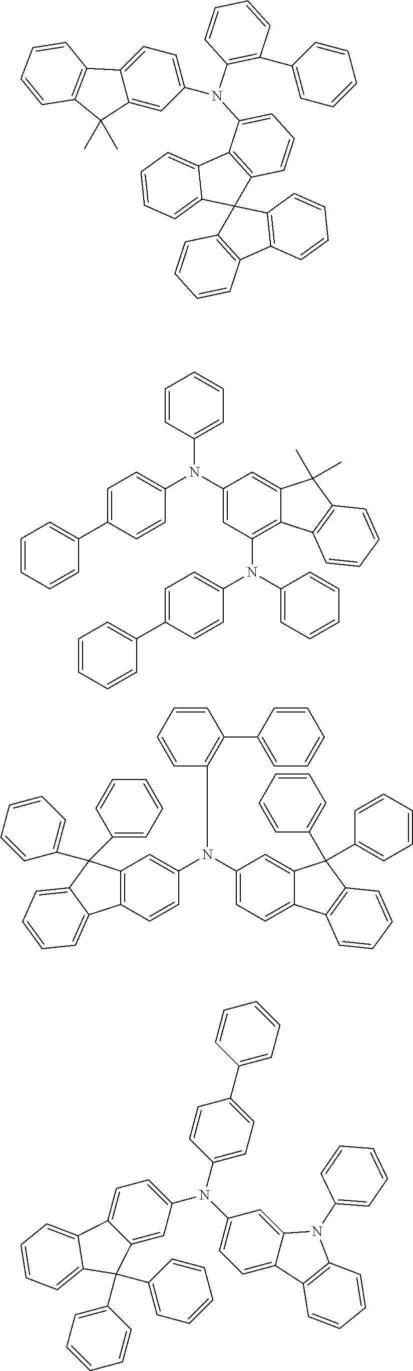 [SMILES: CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2c(N(c3ccccc3)c3ccc(-c4ccccc4)cc3)cc(N(c3ccccc3)c3ccc(-c4ccccc4)cc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1]

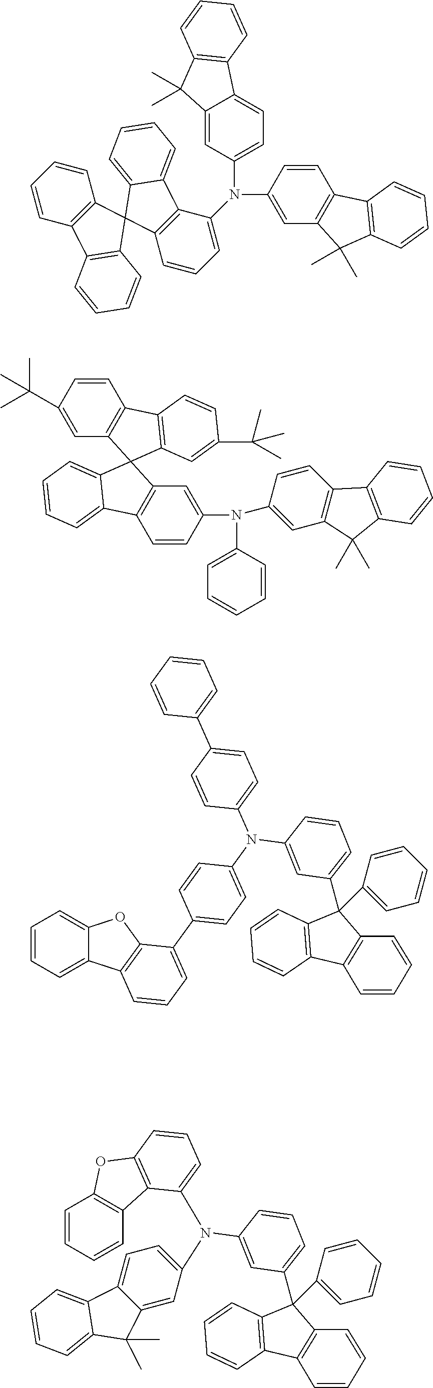 [SMILES: CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2 ; c1ccc(-c2ccc(N(c3ccc(-c4cccc5c4oc4ccccc45)cc3)c3cccc(C4(c5ccccc5)c5ccccc5-c5ccccc54)c3)cc2)cc1 ; CC1(C)c2ccccc2-c2ccc(N(c3cccc(C4(c5ccccc5)c5ccccc5-c5ccccc54)c3)c3cccc4oc5ccccc5c34)cc21]

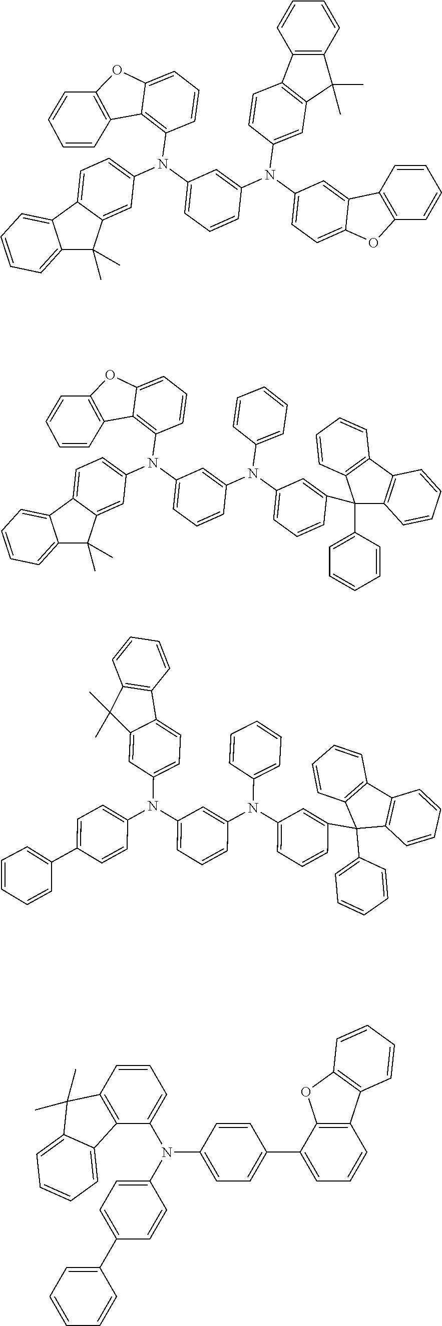 [SMILES: CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21 ; CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21]

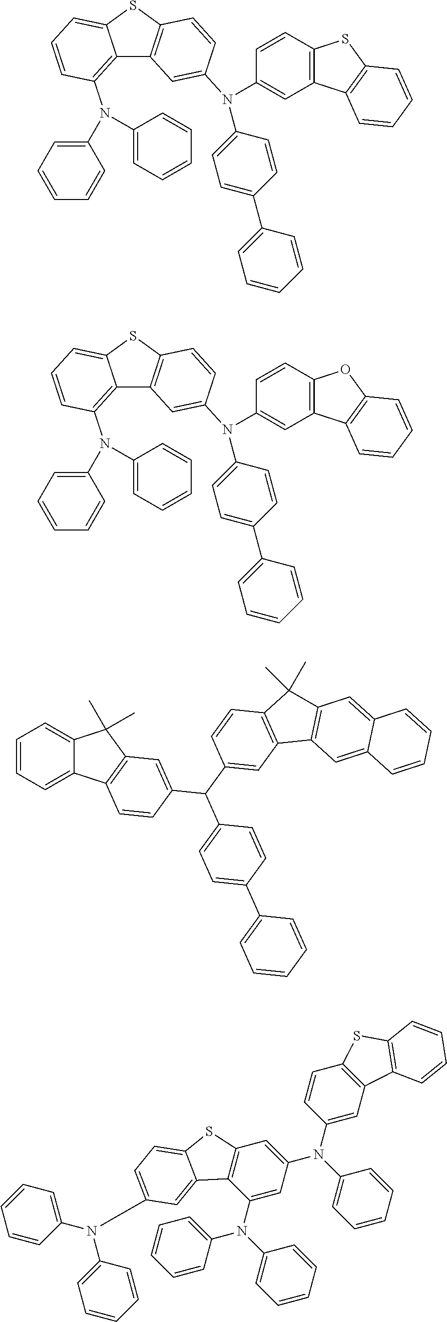

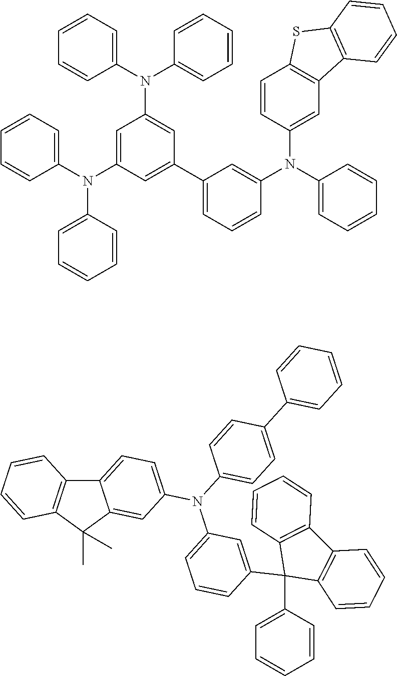 [SMILES: c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In the hole transport region 140, known compounds disclosed in U.S. Pat. No. 5,061,569A, JP1993-009471A, WO1995-009147A1, JP1995-126615A, JP1998-095973A, and the like and compounds similar thereto may be used in addition to the aforementioned compound.

Also, the charge transport region may be, for example, an electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group B may be included in at least one of the electron transport layer and the electron transport auxiliary layer.

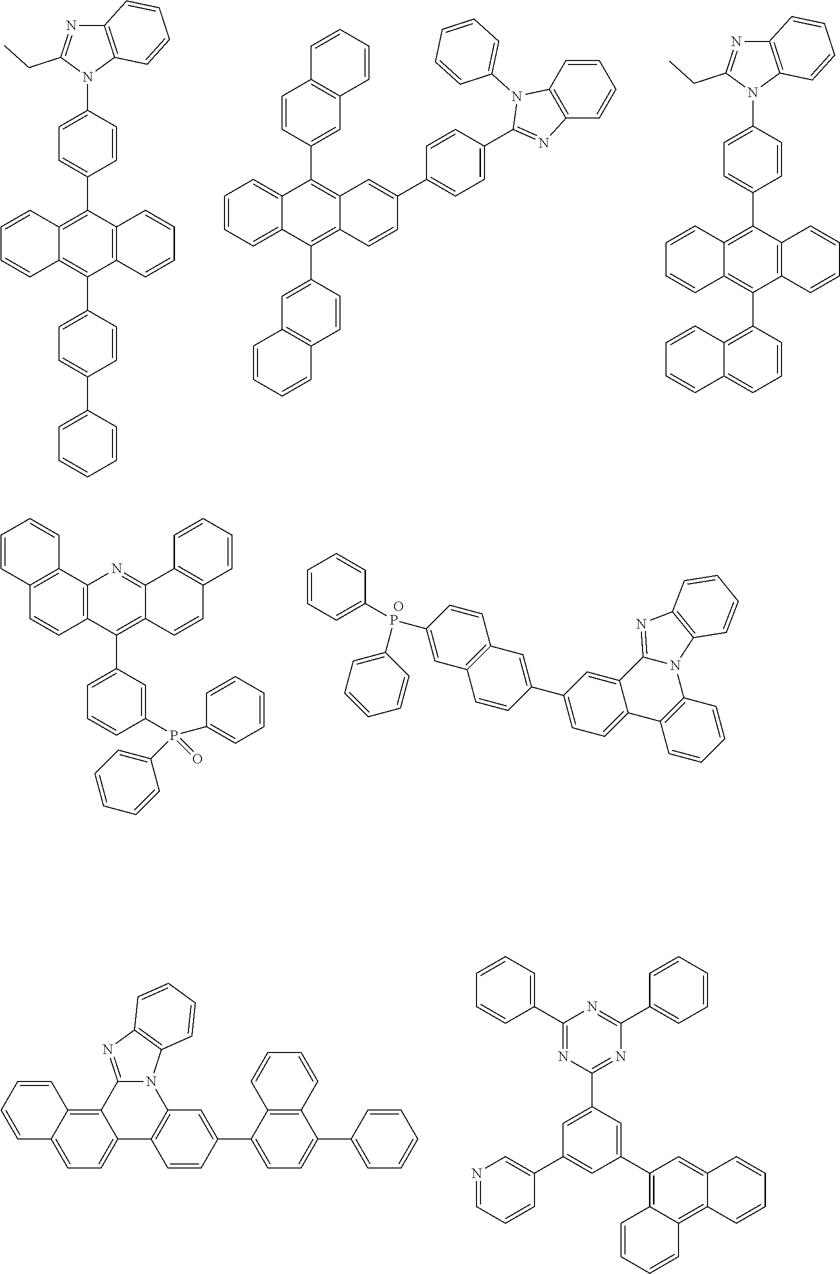

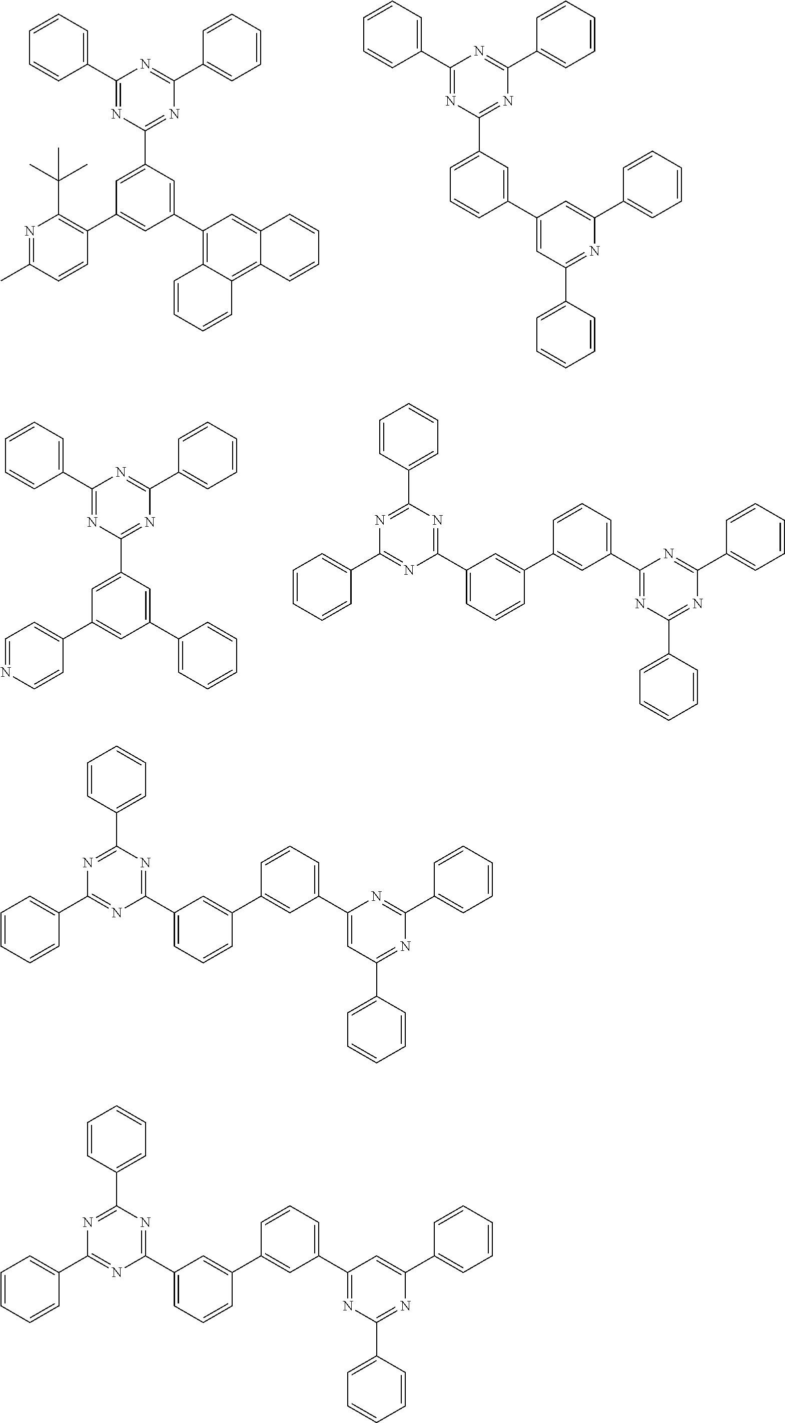 [SMILES: Cc1ccc(-c2cc(-c3nc(-c4ccccc4)nc(-c4ccccc4)n3)cc(-c3cc4ccccc4c4ccccc34)c2)c(C(C)(C)C)n1 ; c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

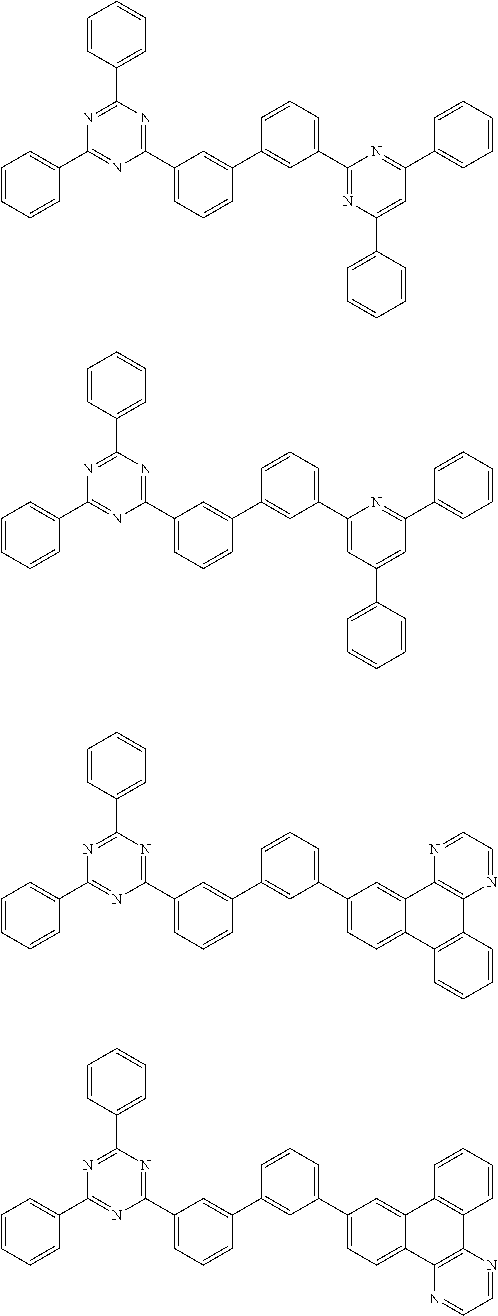 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

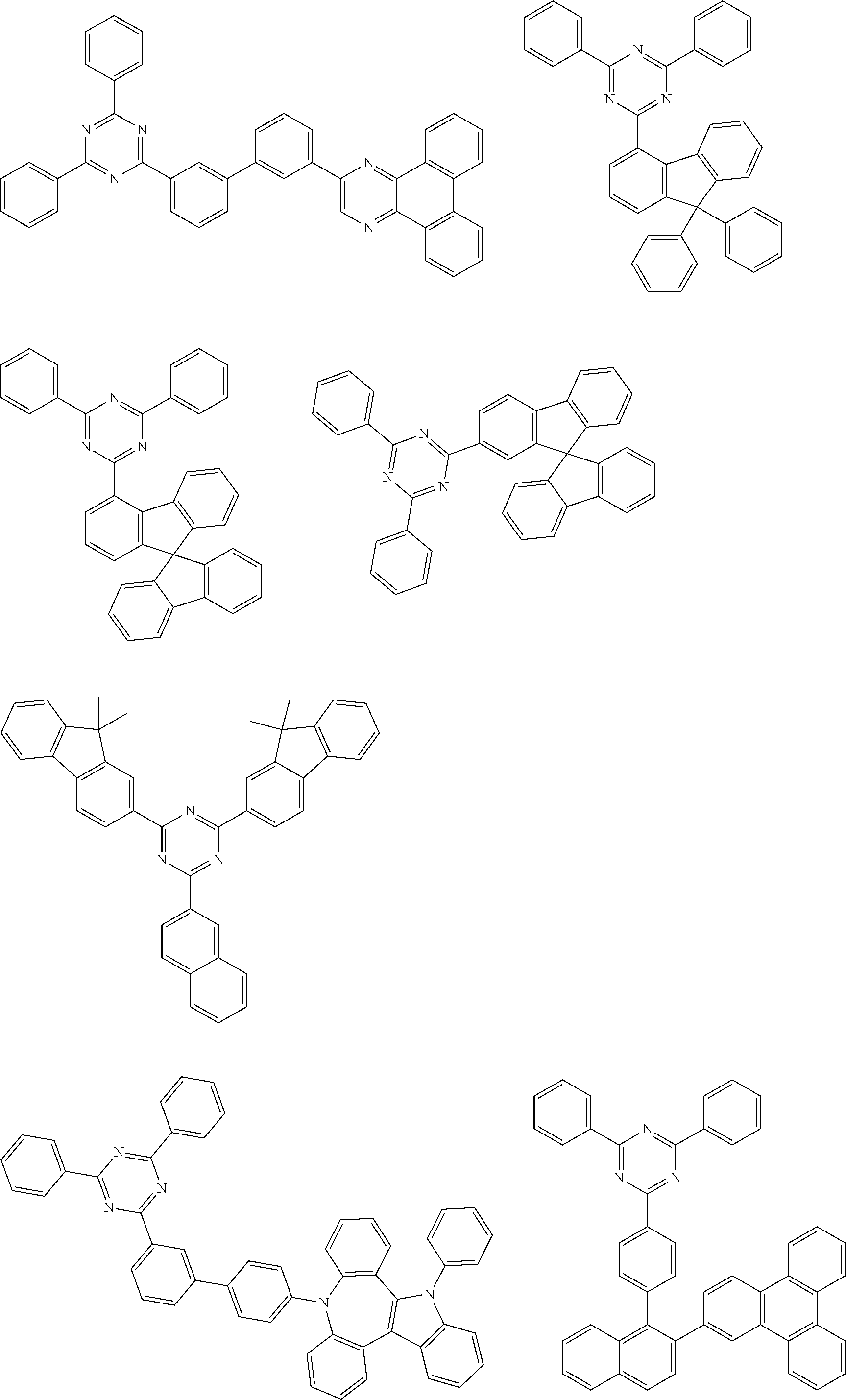

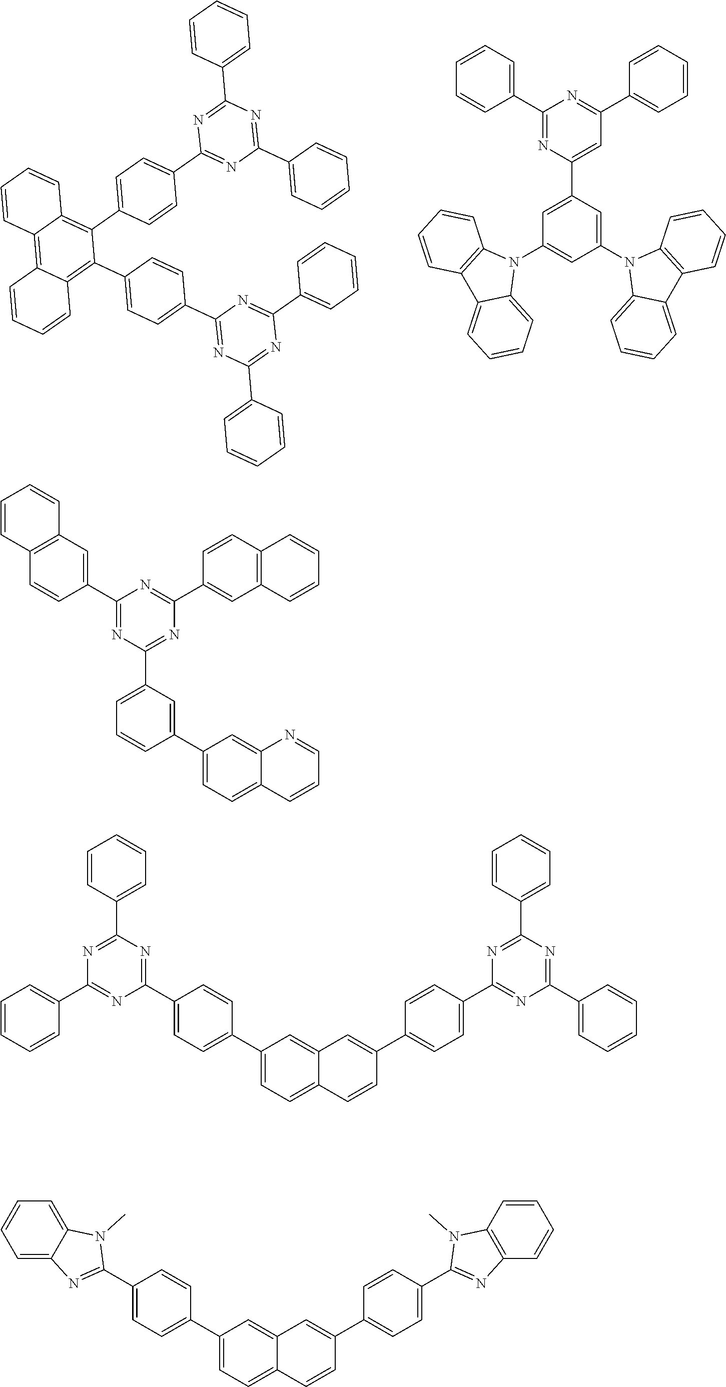 [SMILES: c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1 ; c1ccc(-c2cc(-c3cc(-n4c5ccccc5c5ccccc54)cc(-n4c5ccccc5c5ccccc54)c3)nc(-c3ccccc3)n2)cc1 ; c1cc(-c2ccc3cccnc3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21]

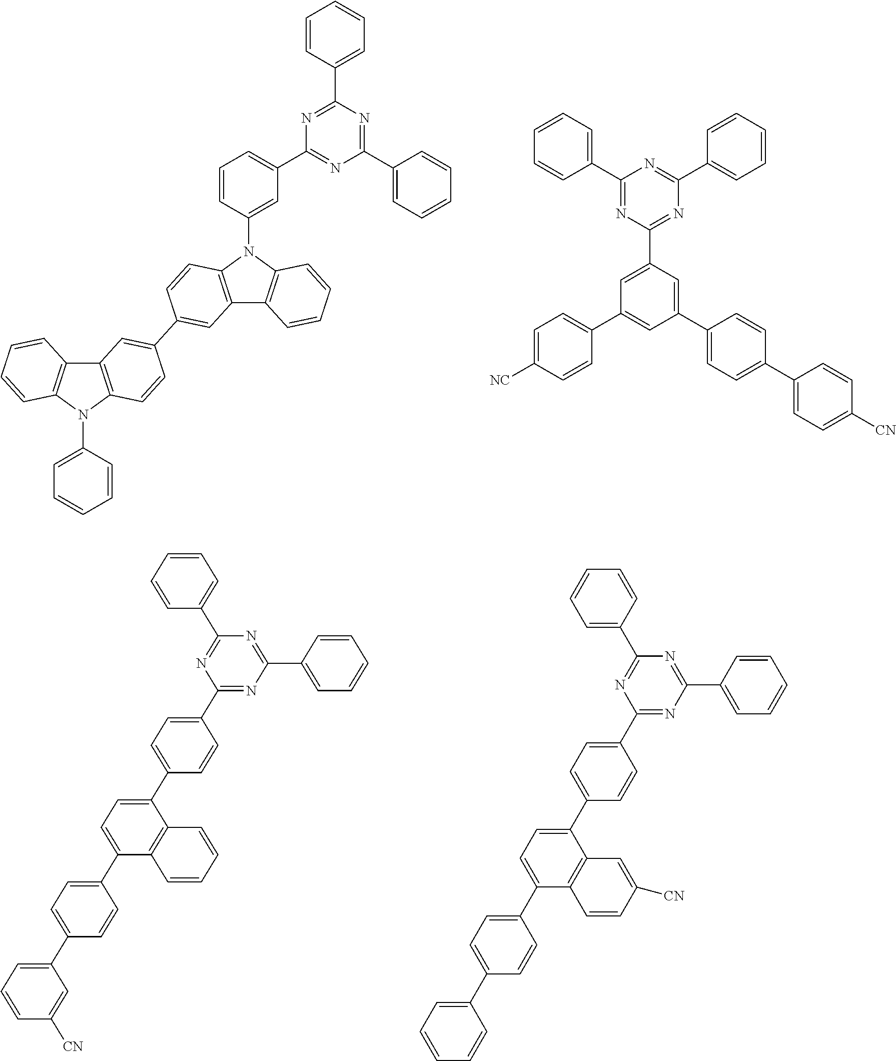 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

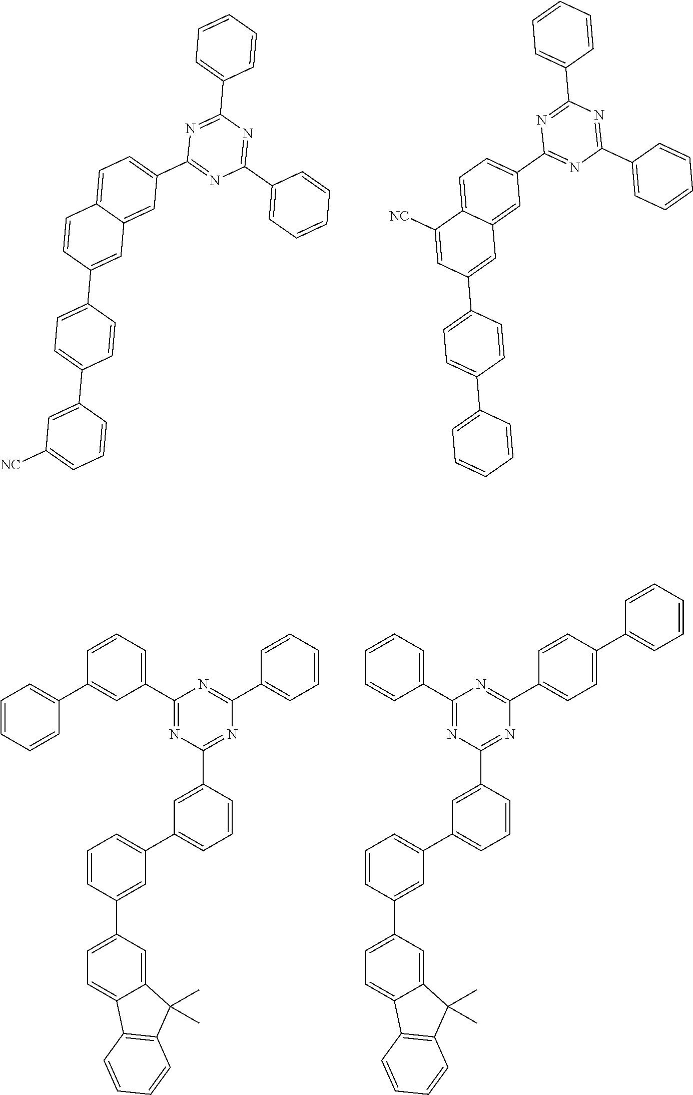

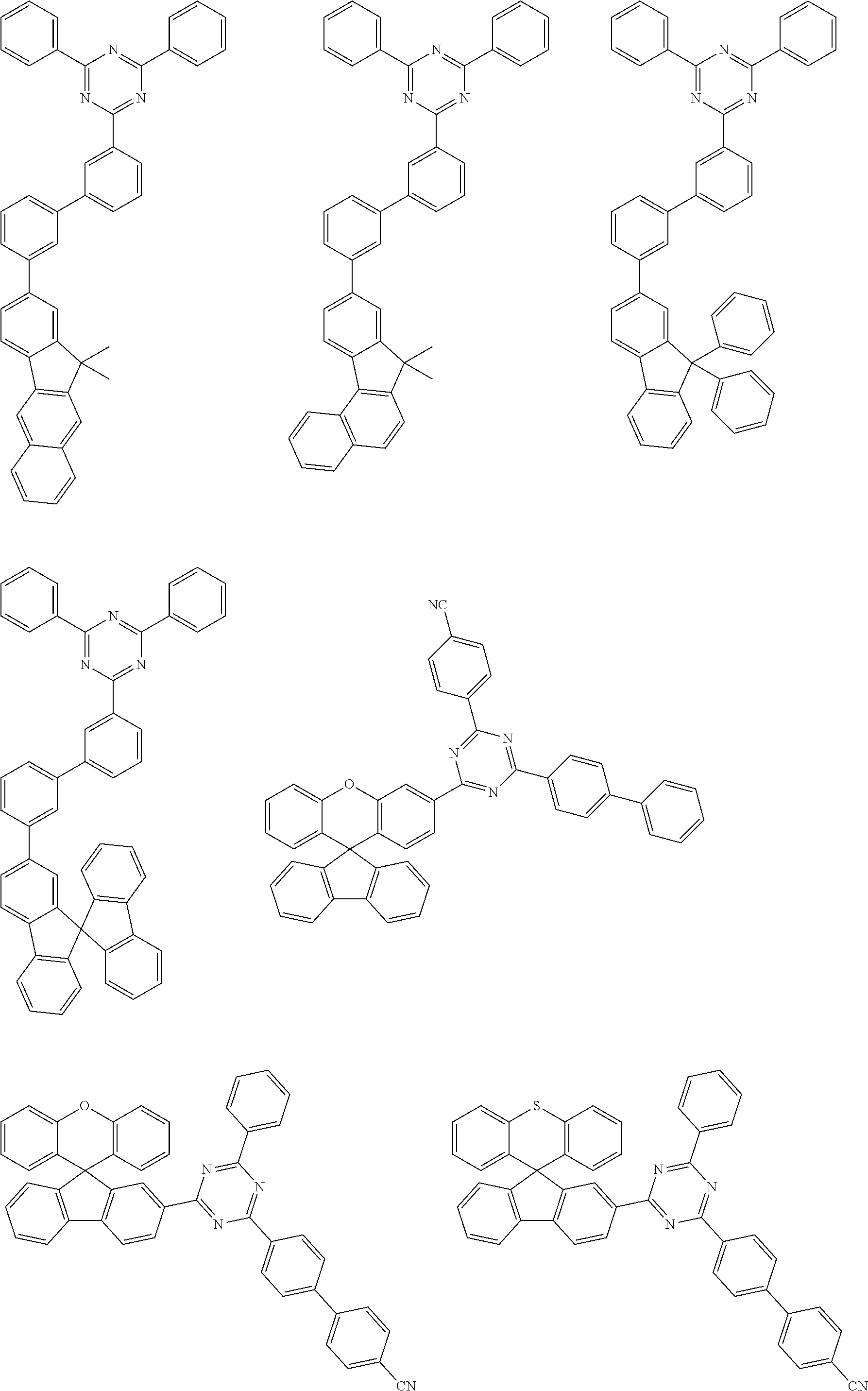

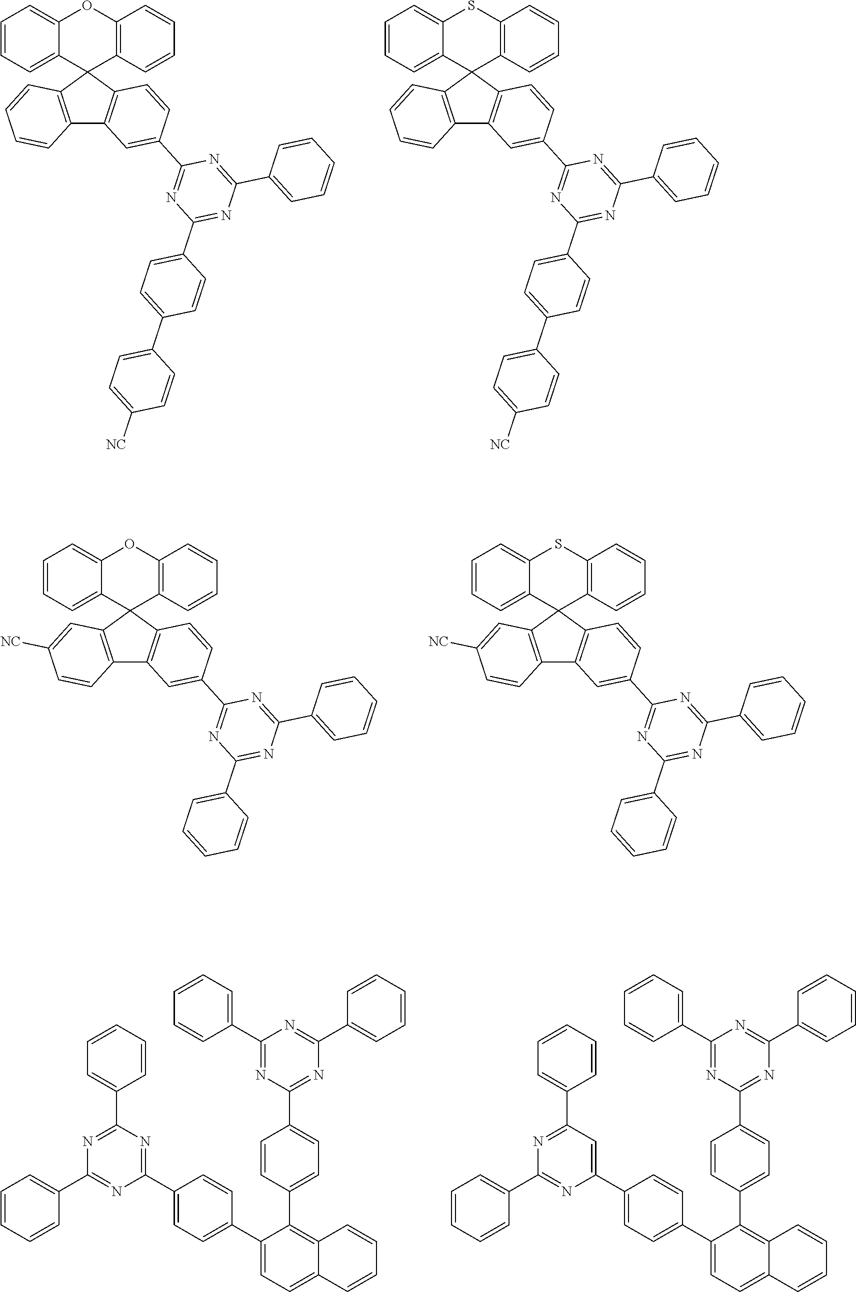

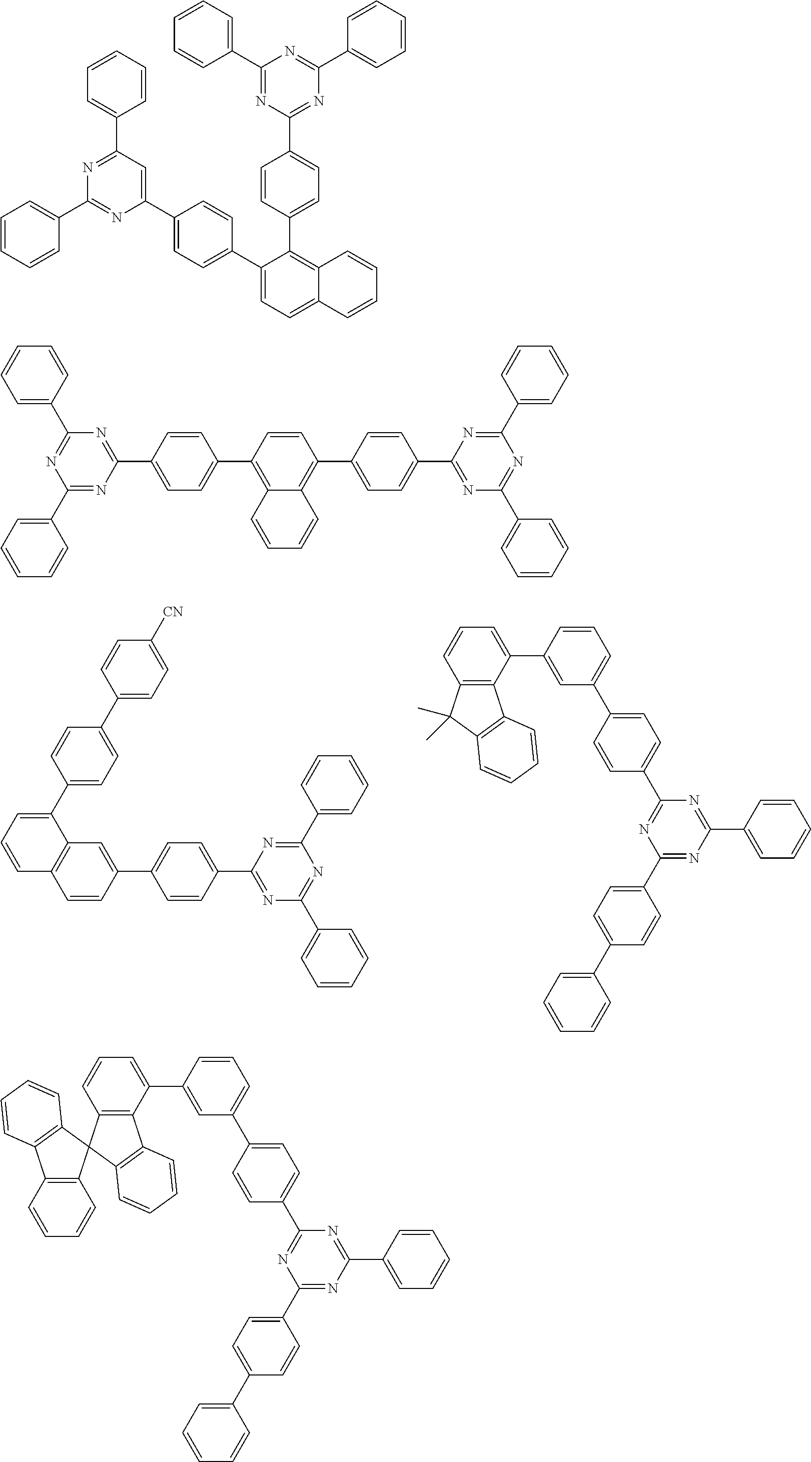 [SMILES: c1ccc(-c2cc(-c3ccc(-c4ccc5ccccc5c4-c4ccc(-c5nc(-c6ccccc6)nc(-c6ccccc6)n5)cc4)cc3)nc(-c3ccccc3)n2)cc1 ; c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1 ; N#Cc1ccc(-c2ccc(-c3cccc4ccc(-c5ccc(-c6nc(-c7ccccc7)nc(-c7ccccc7)n6)cc5)cc34)cc2)cc1 ; CC1(C)c2ccccc2-c2c(-c3cccc(-c4ccc(-c5nc(-c6ccccc6)nc(-c6ccc(-c7ccccc7)cc6)n5)cc4)c3)cccc21 ; c1ccc(-c2ccc(-c3nc(-c4ccccc4)nc(-c4ccc(-c5cccc(-c6cccc7c6-c6ccccc6C76c7ccccc7-c7ccccc76)c5)cc4)n3)cc2)cc1]

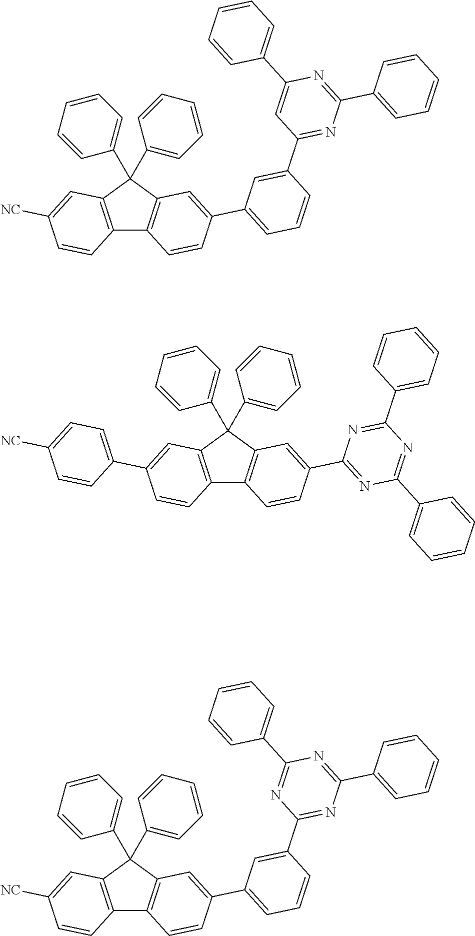 [SMILES: N#Cc1ccc2c(c1)C(c1ccccc1)(c1ccccc1)c1cc(-c3cccc(-c4cc(-c5ccccc5)nc(-c5ccccc5)n4)c3)ccc1-2 ; N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

An embodiment may provide an organic light emitting diode including a light emitting layer as an organic layer.

Another embodiment may provide an organic light emitting diode including a light emitting layer and a hole transport region as an organic layer.

Another embodiment may provide an organic light emitting diode including a light emitting layer and an electron transport region as an organic layer.

As shown in FIG. 1, the organic light emitting diode according to an embodiment of the present invention may include a hole transport region 140 and an electron transport region 150 in addition to the light emitting layer 130 as the organic layer 105.

On the other hand, the organic light emitting diode may further include an electron injection layer (not shown), a hole injection layer (not shown), etc. in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

### DETAILED DESCRIPTION OF THE EMBODIMENTS

Hereinafter, the embodiments are illustrated in more detail with reference to examples. However, these examples are exemplary, and the scope of claims is not limited thereto.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by known methods.

### Preparation of Compound for Organic Optoelectronic Device

The compound presented as a more specific example of the compound of the present invention was synthesized through the following steps.

### Synthesis of First Compound

### Synthesis Example 1: Synthesis of Compound A-6

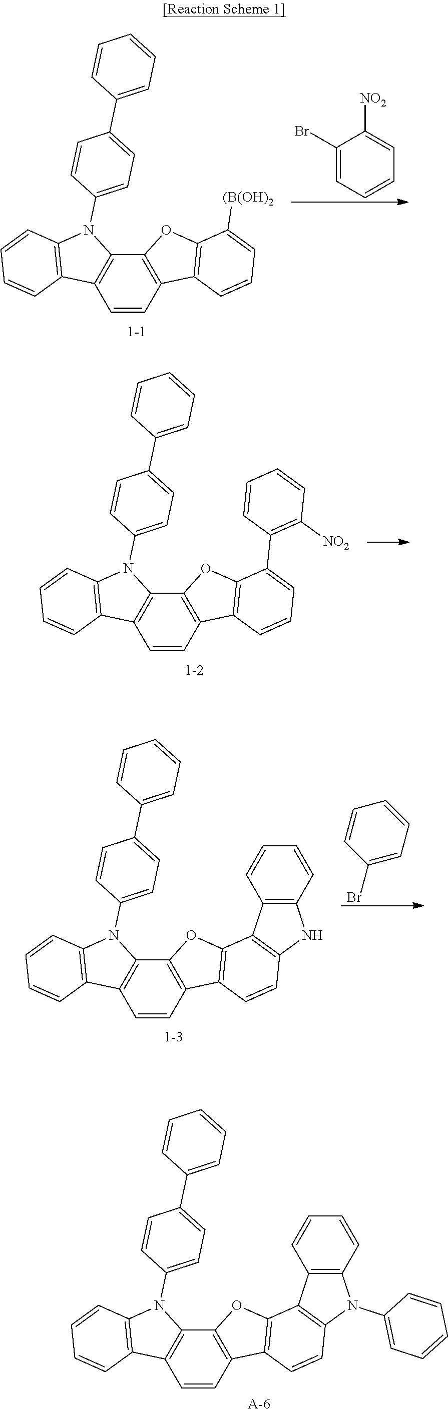

### 1 st Step: Synthesis of Intermediate 1-2

49 g (108.1 mmol) of Intermediate 1-1, 24.02 g (118.91 mmol) of 1-bromo-2-nitrobenzene, 37.35 g (270.24 mmol) of K₂CO₃, and 3.75 g (3.24 mmol) of Pd(PPh₃)₄ 3 were put in a round-bottomed flask and dissolved in 360 ml of toluene and 135 ml of distilled water and then, stirred under reflux at 110° C. for 12 hours. When a reaction was completed, after removing an aqueous layer, an organic layer therein was dissolved in monochlorobenzene (MCB) and silica gel-filtered, and after removing an appropriate amount of an organic solvent, 46.06 g (80%) of Intermediate 1-2 was obtained through recrystallization.

(LC/MS theoretical value: 530.16 g/mol, measured value: M+=531.39 g/mol)

### 2nd Step: Synthesis of Intermediate 1-3

46.06 g (86.81 mmol) of Intermediate 1-2, 68.31 g (260.44 mmol) of triphenylphosphine, and 145 ml of dichlorobenzene (DCB) were put in a round-bottomed flask and then, stirred under reflux at 200° C. for 12 hours. When a reaction was completed, after removing a DCB solvent, the residue was purified through column chromatography. Subsequently, 28.1 g (65%) of Intermediate 1-3 was obtained through recrystallization with MCB.

(LC/MS theoretical value: 498.17 g/mol, measured value: M+=499.31 g/mol)

### 3rd Step: Synthesis of Compound A-6

14 g (28.08 mmol) of Intermediate 1-3, 5.29 g (33.70 mmol) of 4-bromophenyl, 0.51 g (0.56 mmol) of Pd₂(dba)₃, 2.97 g (30.89 mmol) of sodium tert-butoxide, and 0.57 g (2.81 mmol) of tri-tert-butyl phosphine were put in a round-bottomed flask, and 94 ml of xylene was added thereto and then, stirred under reflux at 150° C. for 12 hours. When a reaction was completed, distilled water was added thereto and then, filtered, a solid obtained therefrom was dissolved in MCB and then, silica gel-filtered, and after removing an appropriate amount of an organic solvent, 13.01 g (81%) of Compound A-6 was obtained through recrystallization.

(LC/MS theoretical value: 574.20 g/mol, measured value: M+=575.30 g/mol)

### Synthesis Example 2: Synthesis of Compound A-5

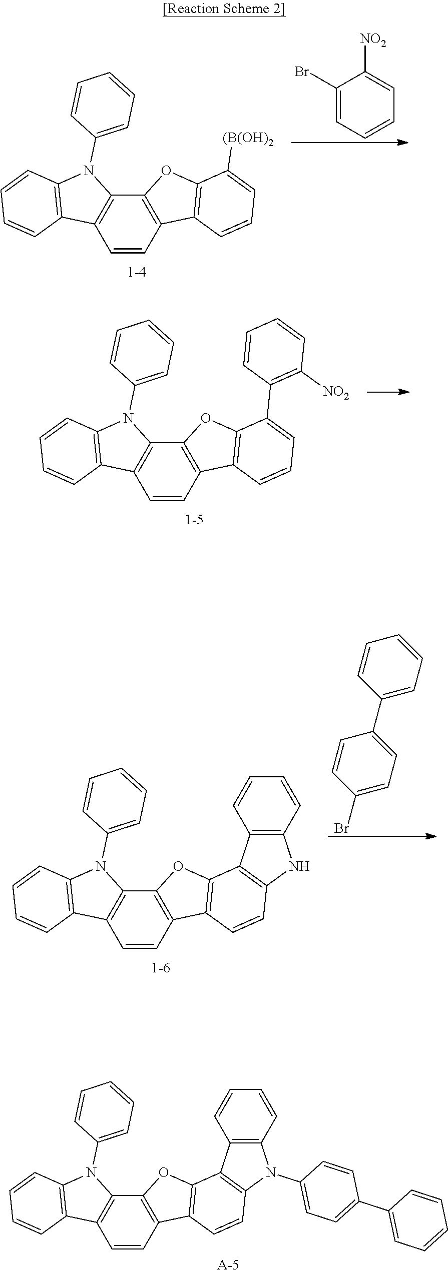

### 1st Step: Synthesis of Intermediate 1-5

55 g (145.81 mmol) of Intermediate 1-4, 30.93 g (153.10 mmol) of 1-bromo-2-nitrobenzene, 50.38 g (364.53 mmol) of K₂CO₃, and 5.05 g (4.37 mmol) of Pd(PPh₃)₄ were put in a round-bottomed flask and then, dissolved in 480 ml of toluene and 180 ml of distilled water and then, stirred under reflux at 110° C. for 12 hours. When a reaction was completed, after removing an aqueous layer, an organic layer therefrom was dissolved in monochlorobenzene (MCB) and then, silica gel-filtered, and after removing an appropriate amount of an organic solvent, 40.1 g (61%) of Intermediate 1-5 was obtained through recrystallization.

(LC/MS theoretical value: 454.13 g/mol, measured value: M+=455.19 g/mol)

### 2nd Step: Synthesis of Intermediate 1-6

40.1 g (88.23 mmol) of Intermediate 1-5, 69.43 g (264.70 mmol) of triphenylphosphine, and 147 ml of dichlorobenzene (DCB) were put in a round-bottomed flask and then, stirred under reflux at 200° C. for 12 hours. When a reaction was completed, after removing a DCB solvent, the residue was purified through column chromatography. Subsequently, 20.1 g (54%) of Intermediate 1-6 was obtained through recrystallization with MCB.

(LC/MS theoretical value: 422.14 g/mol, measured value: M+=423.41 g/mol)

### 3rd Step: Synthesis of Compound A-5

20.1 g (47.58 mmol) of Intermediate 1-6, 13.31 g (57.09 mmol) of 4-bromobiphenyl, 0.87 g (0.95 mmol) of Pd₂(dba)₃, 5.03 g (52.33 mmol) of sodium tert-butoxide, and 0.96 g (4.76 mmol) of tri-tert-butyl phosphine were put in a round-bottomed flask, and 160 ml of xylene was added thereto and then, stirred under reflux at 150° C. for 12 hours. When a reaction was completed, distilled water was added thereto and then, filtered, a solid therefrom was dissolved in MCB and then, filtered with a silica gel, and after removing an appropriate amount of an organic solvent, 20.01 g (73%) of Compound A-5 was obtained through recrystallization.

(LC/MS theoretical value: 574.20 g/mol, measured value: M+=575.41 g/mol)

### Synthesis Example 3: Synthesis of Compound A-7

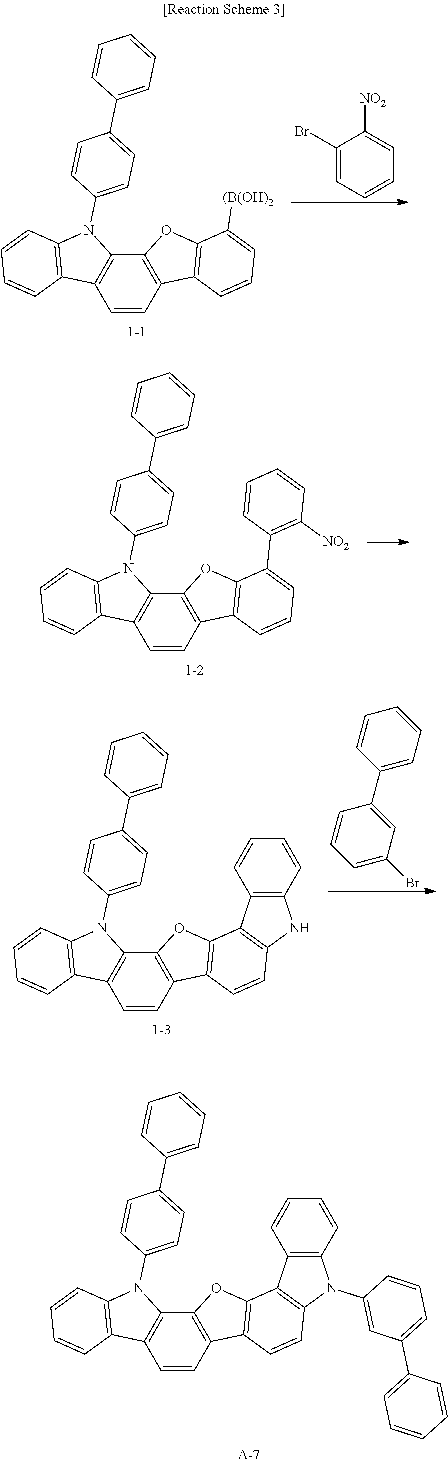

18.0 g (36.10 mmol) of Intermediate 1-3, 10.10 g (43.32 mmol) of 3-bromobiphenyl, 0.66 g (0.72 mmol) of Pd₂(dba)₃, 3.82 g (39.71 mmol) of sodium tert-butoxide, and 0.73 g (3.61 mmol) of tri-tert-butyl phosphine were put in a round-bottomed flask, and 160 ml of xylene was added thereto and then, stirred under reflux at 150° C. for 12 hours. When a reaction was completed, distilled water was added thereto and then, stirred and filtered, a solid therefrom was dissolved in MCB and silica gel-filtered, and after removing an appropriate amount of an organic solvent, 19.01 g (81%) of compound A-7 was obtained through recrystallization.

(LC/MS theoretical value: 650.24 g/mol, measured value: M+=651.31 g/mol)

### Synthesis Example 4: Synthesis of Compound A-1

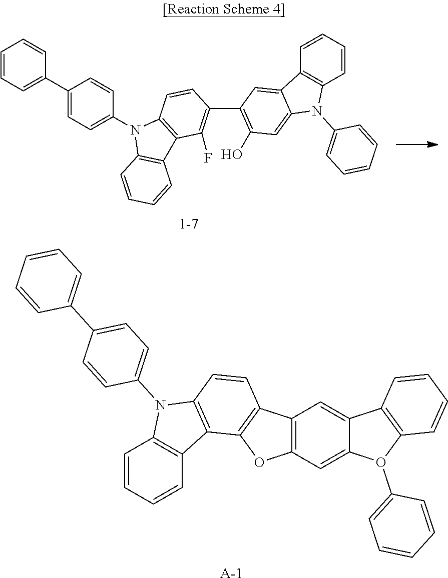

10.0 g (16.81 mmol) of Intermediate 1-7 and 6.97 g (50.43 mmol) of K₂CO₃ were put in a round-bottomed flask, and 21 ml of NMP was added thereto and then, stirred under reflux at 220° C. for 12 hours. When a reaction was completed, distilled water was added thereto and then, stirred and filtered, a solid obtained therefrom was dissolved in MCB and then, filtered with silica gel, and after removing appropriate amount of an organic solvent, 7.73 g (80%) of Compound A-1 was obtained through recrystallization.

(LC/MS theoretical value: 574.20 g/mol, measured value: M+=575.35 g/mol)

### Comparative Synthesis Example 1: Synthesis of Compound Y-1

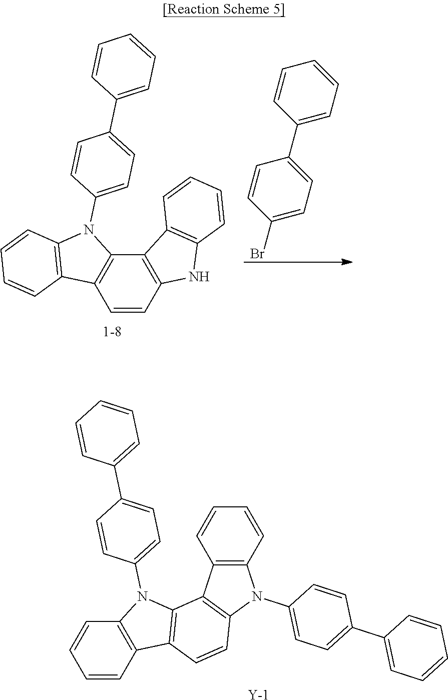

18.0 g (44.06 mmol) of Intermediate 1-8, 8.30 g (52.88 mmol) of 4-bromobiphenyl, 0.81 g (0.88 mmol) of Pd₂(dba)₃, 4.66 g (48.47 mmol) of sodium tert-butoxide, and 0.89 g (4.41 mmol) of tri-tert-butyl phosphine were put in a round-bottomed flask, and 150 ml of xylene was added thereto and then, stirred under reflux at 150° C. for 12 hours. When a reaction was completed, distilled water was added thereto and then, stirred and filtered, a solid obtained therefrom was dissolved in MCB and filtered with silica gel, and after removing an appropriate amount of an organic solvent, 20.01 g (81%) of Compound Y-1 was obtained through recrystallization.

(LC/MS theoretical value: 574.67 g/mol, measured value: M+=575.85 g/mol)

### Comparative Synthesis Example 2: Synthesis of Compound Y-2

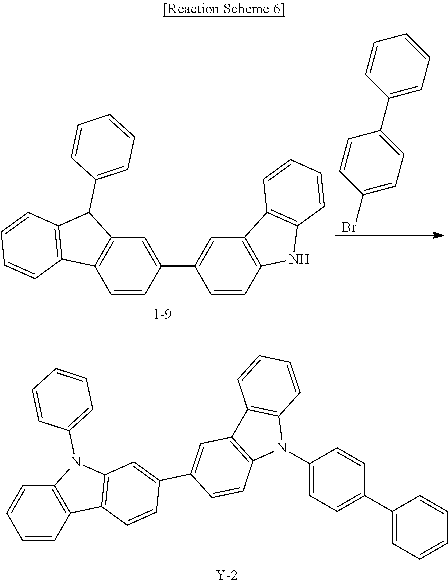

18.0 g (44.06 mmol) of Intermediate 1-9, 8.30 g (52.88 mmol) of 4-bromobiphenyl, 0.81 g (0.88 mmol) of Pd₂(dba)₃, 4.66 g (48.47 mmol) of sodium tert-butoxide, and 0.89 g (4.41 mmol) of tri-tert-butyl phosphine were put in a round-bottomed flask, and 150 ml of xylene was added thereto and then, stirred under reflux at 150° C. for 12 hours. When a reaction was completed, distilled water was added thereto and then, stirred and filtered, a solid obtained therefrom was dissolved in MCB and then, filtered with silica gel, and after removing an organic solvent, 18.53 g (75%) of Compound Y-2 was obtained through recrystallization.

(LC/MS theoretical value: 574.67 g/mol, measured value: M+=575.81 g/mol)

### Synthesis of Second Compound

### Synthesis Example 5: Synthesis of Compound D-8

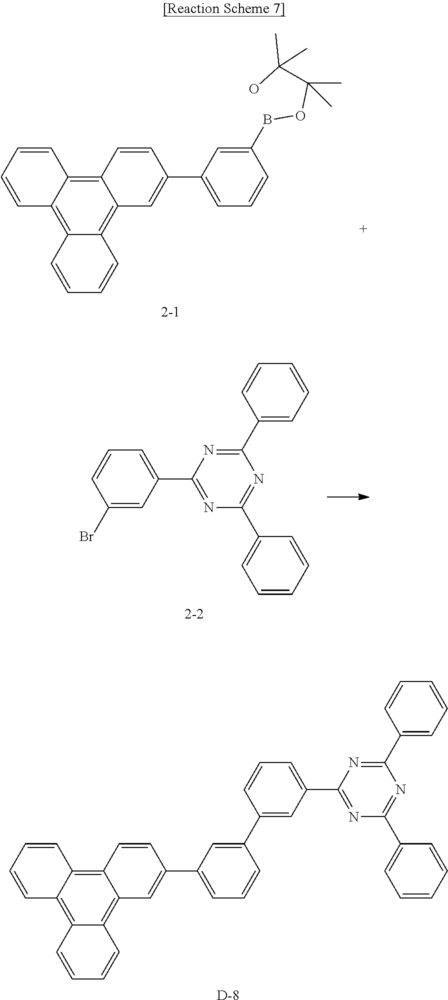

10 g (23.24 mmol) of Intermediate 2-1, 9.93 g (25.57 mmol) of Intermediate 2-2, 8.03 g (58.11 mmol) of K₂CO₃, and 0.81 g (0.70 mmol) of Pd(PPh₃)₄ were put in a round-bottomed flask and dissolved in 78 ml of toluene and 30 ml of distilled water and then, stirred under reflux at 110° C. for 12 hours. When a reaction was completed, after removing an aqueous layer, an organic layer therefrom was dissolved in DCB and then, filtered with silica gel, and after removing an appropriate amount of an organic solvent, 11.52 g (81%) of Compound D-8 was obtained through recrystallization.

(LC/MS theoretical value: 611.24 g/mol, measured value: M+=612.19 g/mol)

### Synthesis Example 6: Synthesis of D-33

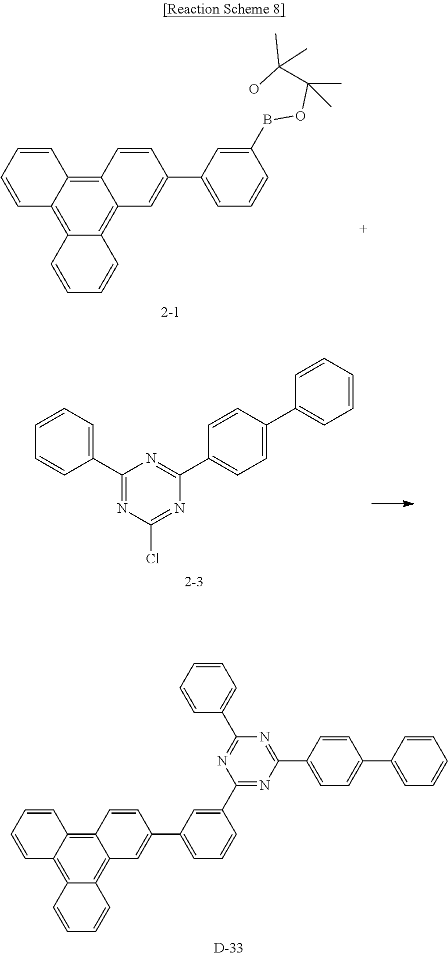

10 g (23.24 mmol) of Intermediate 2-1, 8.79 g (25.57 mmol) of Intermediate 2-3, 8.03 g (58.11 mmol) of K₂CO₃, and 0.81 g (0.70 mmol) of Pd(PPh₃)₄ were put in a round-bottomed flask and dissolved in 78 ml of toluene and 30ml of distilled water and then, stirred under reflux at 110° C. for 12 hours. When a reaction was completed, after removing an aqueous layer, an organic layer was dissolved in DCB and then, silica gel-filtered, and after removing an appropriate amount of an organic solvent, 11.80 g (83%) of Compound D-33 was obtained through recrystallization.

(LC/MS theoretical value: 611.24 g/mol, measured value: M+=612.18 g/mol)

### Synthesis Example 7: Synthesis of D-54

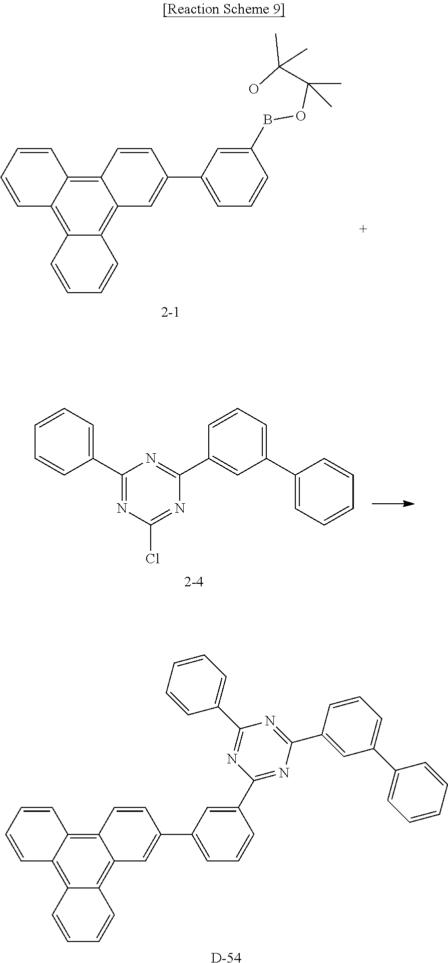

10 g (23.24 mmol) of Intermediate 2-1, 8.79 g (25.57 mmol) of Intermediate 2-4, 8.03 g (58.11 mmol) of K₂CO₃, and 0.81 g (0.70 mmol) of Pd(PPh₃)₄ were put in a round-bottomed flask and dissolved in 78 ml of toluene and 30 ml of distilled water and then, stirred under reflux at 110° C. for 12 hours. When a reaction was completed, after removing an aqueous layer, an organic layer therefrom was dissolved in DCB and then, filtered with silica gel, and after removing an appropriate amount of an organic solvent, 10.95 g (79%) of Compound D-54 was obtained through recrystallization.

(LC/MS theoretical value: 611.24 g/mol, measured value: M+=612.15 g/mol)

### Manufacture of Organic Light Emitting Diode

### Example 1

A glass substrate coated with ITO (Indium tin oxide) was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited to on the hole injection layer to form a 1350 Å-thick hole transport layer. On the hole transport layer, a 350 Å-thick hole transport auxiliary layer was formed by depositing Compound B. On the hole transport auxiliary layer, a 400 Å-thick light emitting layer was formed by simultaneously vacuum-depositing Compound A-6 of Synthesis Example 1 and Compound D-8 of Synthesis Example 5 as a host and doping 10 wt % of PhGD as a dopant and the Compound A-6 and Compound D-8 were used in a weight ratio of 4:6. Subsequently, Compound C was deposited on the light emitting layer to form a 50 Å-thick electron transport auxiliary layer, and Compound D and Liq were simultaneously vacuum-deposited at a weight ratio of 1:1 to form a 300 Å-thick electron transport layer. LiQ (15 Å) and Al (1200 Å) were sequentially vacuum-deposited on the electron transport layer to form a cathode, thereby manufacturing an organic light emitting diode.

ITO/Compound A (3% NDP-9 doping, 100 Å)/Compound A (1350 Å)/Compound B (350 Å)/EML [90 wt % of Compound A-6:Compound D-8=weight ratio of 4:6), 10 wt % of dopant (PhGD)] (400 Å)/Compound C (50 Å)/Compound D:LiQ (300 Å)/LiQ (15 Å)/Al (1200 Å).

Compound A: N-(biphenyl-4-yl)-9,9-dimethyl-N-(4-(9-phenyl-9H-carbazol-3-yl)phenyl)-9H-fluoren-2-amine

Compound B: N,N-bis(9,9-dimethyl-9H-fluoren-4-yl)-9,9-spirobi(fluorene)-2-amine

Compound C: 2-[3′-(9,9-dimethyl-9H-fluoren-2-yl)[1,1′-biphenyl]-3-yl]-4,6-diphenyl-1,3,5-triazine

Compound D: 2-[4-[4-(4′-cyano-1,1′-biphenyl-4-yl)-1-naphthyl]phenyl]-4,6-diphenyl-1,3,5-triazine

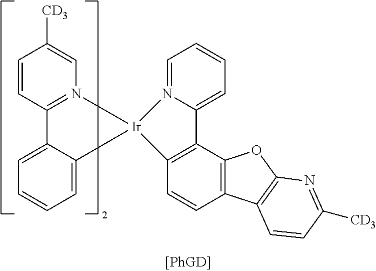

### Examples 2 to 12 and Comparative Examples 1 and 2

Diodes of Examples 2 to 12 and Comparative Examples 1 and 2 were manufactured in the same manner as in Example 1, except that the host composition was changed as shown in Table 1.

### Evaluations

The driving voltages of the organic light emitting diodes according to Examples 1 to 12 and Comparative Examples 1 and 2 were measured as follows, and the results are shown in Table 1.

### Measurement of Driving Voltage

The driving voltage of each diode at 15 mA/cm² was measured using a current-voltmeter (Keithley 2400) to obtain results.

The relative comparison values with the driving voltage of Comparative Example 3 are shown in Table 1.

TABLE 1

| | First | Second | Driving |
|---|---|---|---|
| | host | host | voltage (%) |
| Example 1 | A-6 | D-8 | 90 |
| Example 2 | A-5 | D-8 | 92 |
| Example 3 | A-7 | D-8 | 92 |
| Example 4 | A-1 | D-8 | 91 |
| Example 5 | A-6 | D-33 | 89 |
| Example 6 | A-5 | D-33 | 91 |
| Example 7 | A-7 | D-33 | 91 |
| Example 8 | A-1 | D-33 | 90 |
| Example 9 | A-6 | D-54 | 91 |
| Example 10 | A-5 | D-54 | 93 |
| Example 11 | A-7 | D-54 | 93 |
| Example 12 | A-1 | D-54 | 92 |
| Comparative Example 1 | Y-1 | D-8 | 100 |
| Comparative Example 2 | Y-2 | D-8 | 101 |

Referring to Table 1, the driving voltages of the compositions according to the present invention are greatly improved compared to the compositions according to Comparative Examples.

While this invention has been described in connection with what is presently considered to be practical embodiments, it is to be understood that the invention is not limited to the disclosed embodiments, but, on the contrary, is intended to cover various modifications and equivalent arrangements included within the spirit and scope of the appended claims.

1. A composition for an organic optoelectronic device, the composition comprising:
a first compound represented by a combination of Chemical Formula 1 and Chemical Formula 2, and
a second compound represented by Chemical Formula 3: 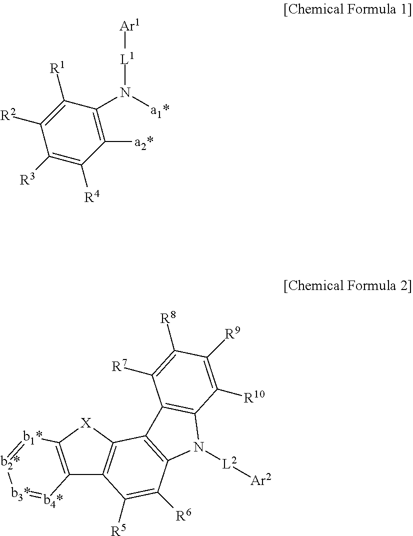
wherein, in Chemical Formula 1 and Chemical Formula 2,
a1* and a2* are each a linking carbon (C),
b1* to b4* are each independently CR^{a} or a linking carbon (C),
a1* and a2* are each linked to adjacent two of b1* to b4*,
R^{a} and R¹ to R¹⁰ are each independently hydrogen, deuterium, a cyano group, a halogen, a substituted or unsubstituted C1 to C30 alkyl group, or a substituted or unsubstituted C6 to C30 aryl group,
L¹ and L² are each independently a single bond or a substituted or unsubstituted C6 to C20 arylene group,
Ar¹ and Ar² are each independently a substituted or unsubstituted C6 to C30 aryl group, and
X is O or S; 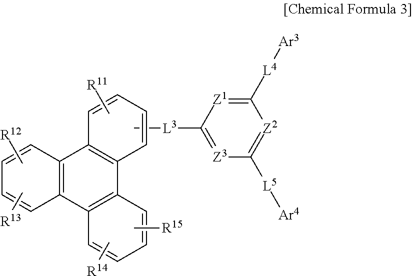
wherein, in Chemical Formula 3,
Z¹ to Z³ are each independently N or C-L^{b}-R^{b},
at least two of Z¹ to Z³ are N,
L^{b} and L³ to L⁵ are each independently a single bond or a substituted or unsubstituted C6 to C20 arylene group,
Ar³ and Ar⁴ are each independently a substituted or unsubstituted C6 to C30 aryl group or a substituted or unsubstituted C2 to C30 heterocyclic group, and
R^{b} and R¹¹ to R¹⁵ are each independently hydrogen, deuterium, a cyano group, a halogen, a substituted or unsubstituted C1 to C30 alkyl group, or a substituted or unsubstituted C6 to C30 aryl group.

2. The composition for an organic optoelectronic device of claim 1, wherein:
the first compound is represented by any one of Chemical Formula 1A to Chemical Formula 1F: 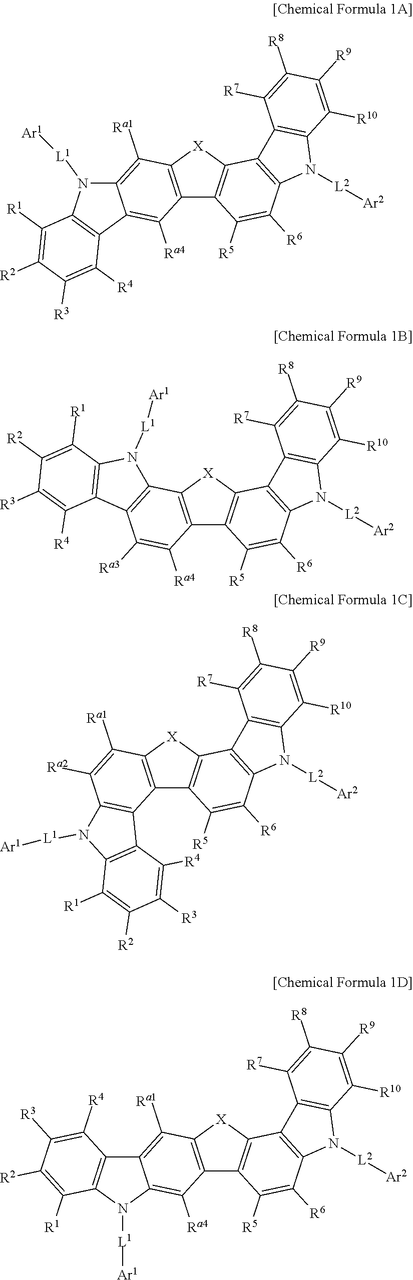 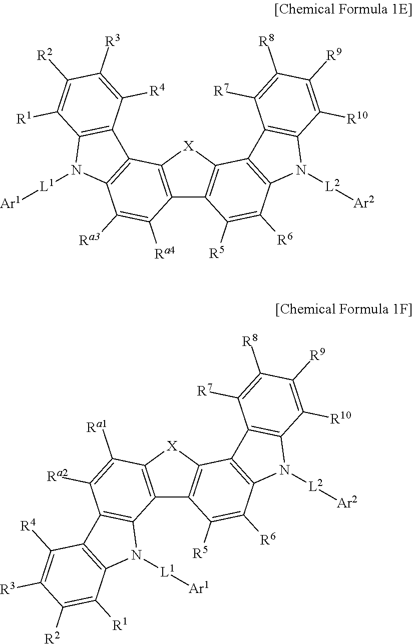
in Chemical Formula 1A to Chemical Formula 1F,
R¹ to R¹⁰, L¹, L², Ar¹, Ar², and X are defined the same as those of Chemical Formula 1 and Chemical Formula 2, and
R^{a1} to R^{a4} are each independently defined the same as R^{a} of Chemical Formula 1 and Chemical Formula 2.

3. The composition for an organic optoelectronic device of claim 2, wherein the first compound is represented by Chemical Formula 1A or Chemical Formula 1B.

4. The composition for an organic optoelectronic device of claim 1, wherein Ar¹ and Ar² are each independently a substituted or unsubstituted phenyl group, a substituted or unsubstituted biphenyl group, or a substituted or unsubstituted naphthyl group.

5. The composition for an organic optoelectronic device of claim 1, wherein:
Ar¹ and Ar² are each independently one a group of Group I: 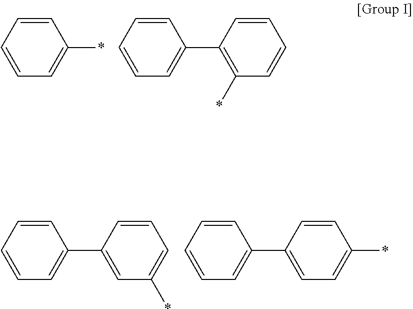
in Group I, * is a linking point.

6. The composition for an organic optoelectronic device of claim 1, wherein the first compound is a compound of Group 1:

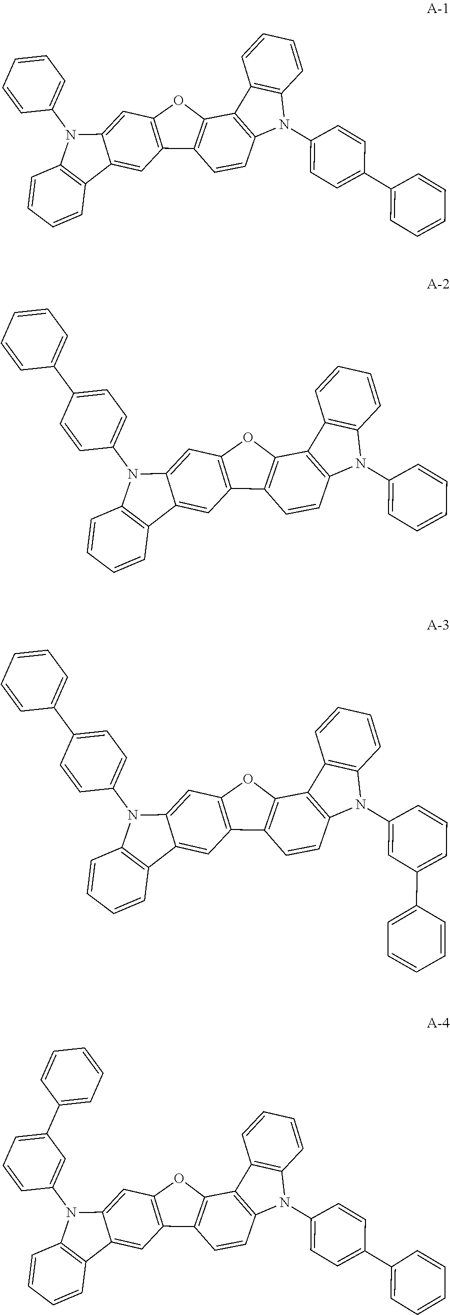 [SMILES: c1ccc(-c2ccc(-n3c4ccccc4c4c5oc6cc7c(cc6c5ccc43)c3ccccc3n7-c3ccccc3)cc2)cc1 ; c1ccc(-c2ccc(-n3c4ccccc4c4cc5c(cc43)oc3c5ccc4c3c3ccccc3n4-c3ccccc3)cc2)cc1 ; c1ccc(-c2ccc(-n3c4ccccc4c4cc5c(cc43)oc3c5ccc4c3c3ccccc3n4-c3cccc(-c4ccccc4)c3)cc2)cc1 ; c1ccc(-c2ccc(-n3c4ccccc4c4c5oc6cc7c(cc6c5ccc43)c3ccccc3n7-c3cccc(-c4ccccc4)c3)cc2)cc1]

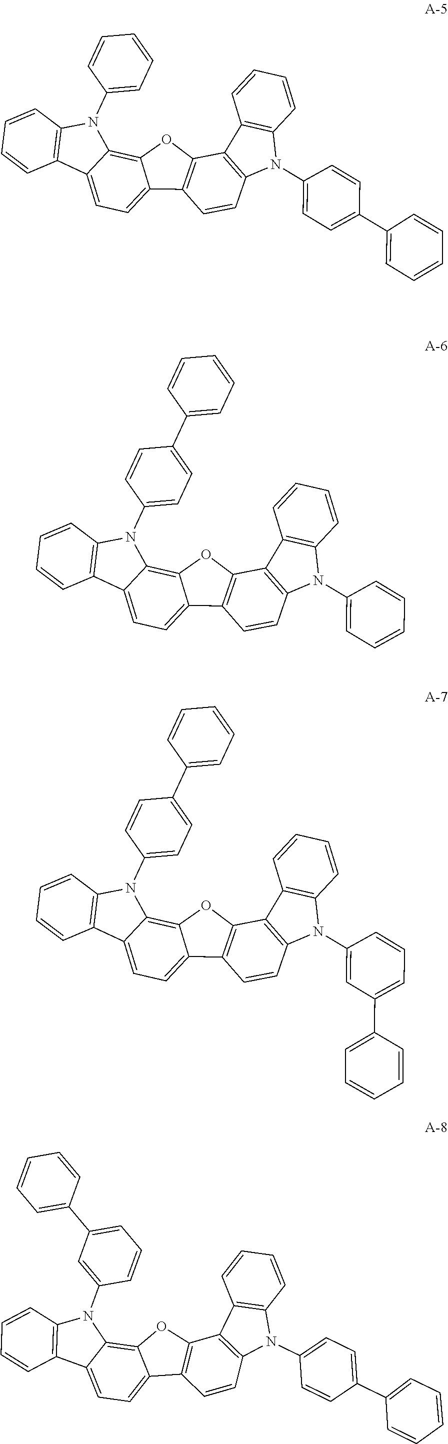 [SMILES: c1ccc(-c2ccc(-n3c4ccccc4c4c5oc6c(ccc7c8ccccc8n(-c8ccccc8)c76)c5ccc43)cc2)cc1 ; c1ccc(-c2ccc(-n3c4ccccc4c4ccc5c6ccc7c(c8ccccc8n7-c7ccccc7)c6oc5c43)cc2)cc1 ; c1ccc(-c2ccc(-n3c4ccccc4c4ccc5c6ccc7c(c8ccccc8n7-c7cccc(-c8ccccc8)c7)c6oc5c43)cc2)cc1 ; c1ccc(-c2ccc(-n3c4ccccc4c4c5oc6c(ccc7c8ccccc8n(-c8cccc(-c9ccccc9)c8)c76)c5ccc43)cc2)cc1]

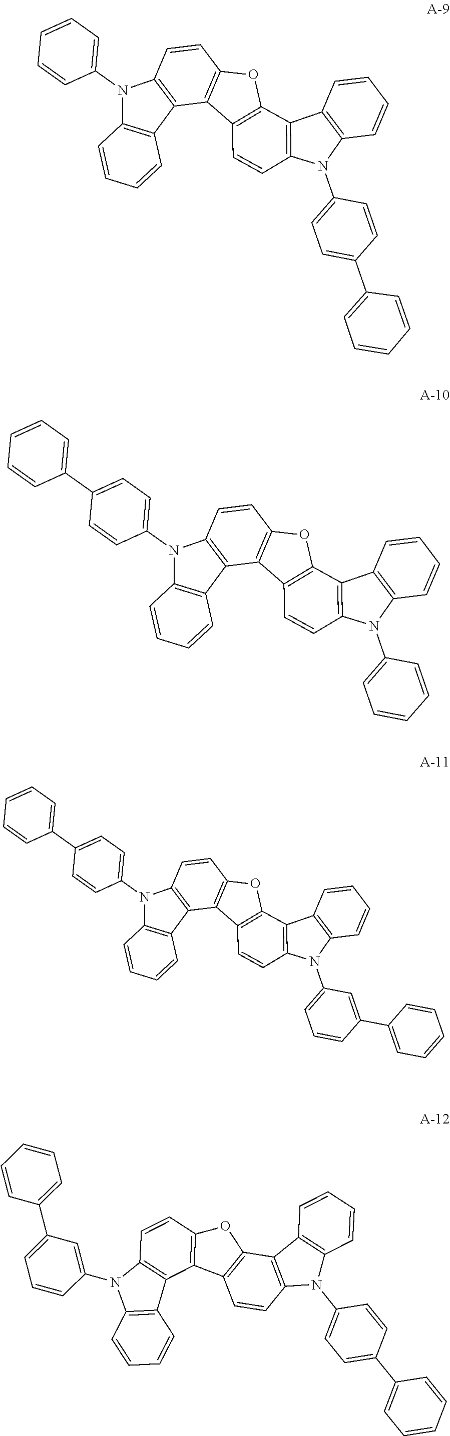 [SMILES: c1ccc(-c2ccc(-n3c4ccccc4c4c5oc6ccc7c(c8ccccc8n7-c7ccccc7)c6c5ccc43)cc2)cc1 ; c1ccc(-c2ccc(-n3c4ccccc4c4c5c(ccc43)oc3c5ccc4c3c3ccccc3n4-c3ccccc3)cc2)cc1 ; c1ccc(-c2ccc(-n3c4ccccc4c4c5c(ccc43)oc3c5ccc4c3c3ccccc3n4-c3cccc(-c4ccccc4)c3)cc2)cc1 ; c1ccc(-c2ccc(-n3c4ccccc4c4c5oc6ccc7c(c8ccccc8n7-c7cccc(-c8ccccc8)c7)c6c5ccc43)cc2)cc1]

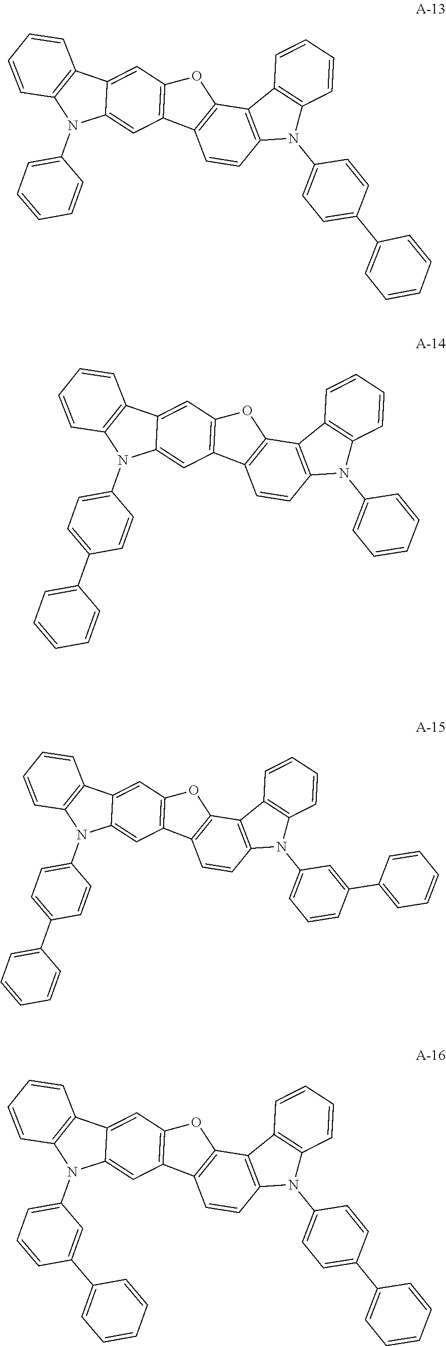 [SMILES: c1ccc(-c2ccc(-n3c4ccccc4c4c5oc6cc7c8ccccc8n(-c8ccccc8)c7cc6c5ccc43)cc2)cc1 ; c1ccc(-c2ccc(-n3c4ccccc4c4cc5oc6c(ccc7c6c6ccccc6n7-c6ccccc6)c5cc43)cc2)cc1 ; c1ccc(-c2ccc(-n3c4ccccc4c4cc5oc6c(ccc7c6c6ccccc6n7-c6cccc(-c7ccccc7)c6)c5cc43)cc2)cc1 ; c1ccc(-c2ccc(-n3c4ccccc4c4c5oc6cc7c8ccccc8n(-c8cccc(-c9ccccc9)c8)c7cc6c5ccc43)cc2)cc1]

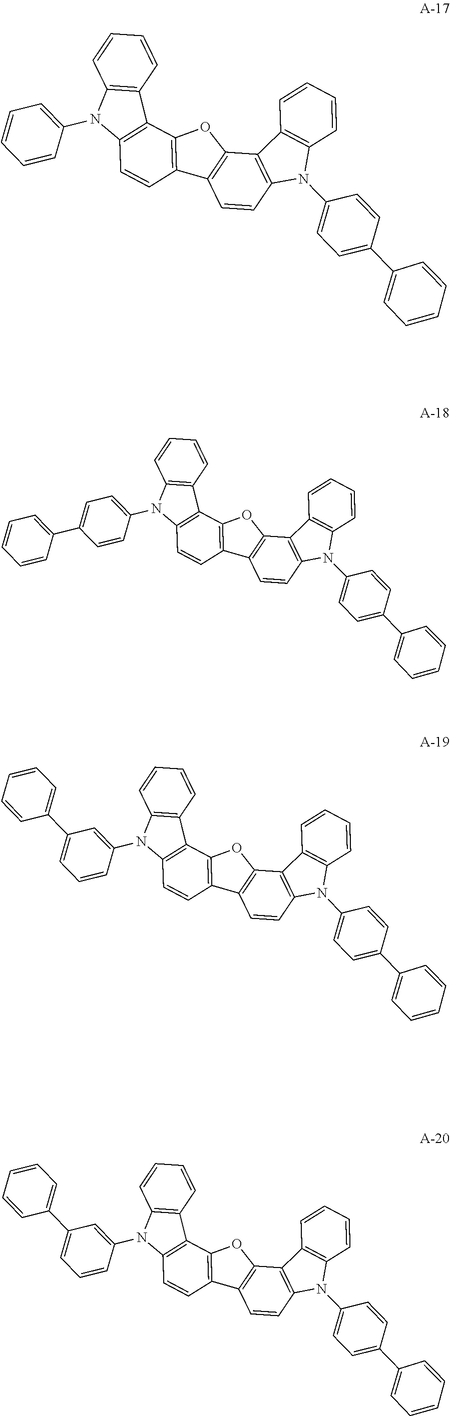 [SMILES: c1ccc(-c2ccc(-n3c4ccccc4c4c5oc6c(ccc7c6c6ccccc6n7-c6ccccc6)c5ccc43)cc2)cc1 ; c1ccc(-c2ccc(-n3c4ccccc4c4c5oc6c(ccc7c6c6ccccc6n7-c6ccc(-c7ccccc7)cc6)c5ccc43)cc2)cc1 ; c1ccc(-c2ccc(-n3c4ccccc4c4c5oc6c(ccc7c6c6ccccc6n7-c6cccc(-c7ccccc7)c6)c5ccc43)cc2)cc1 ; c1ccc(-c2ccc(-n3c4ccccc4c4c5oc6c(ccc7c6c6ccccc6n7-c6cccc(-c7ccccc7)c6)c5ccc43)cc2)cc1]

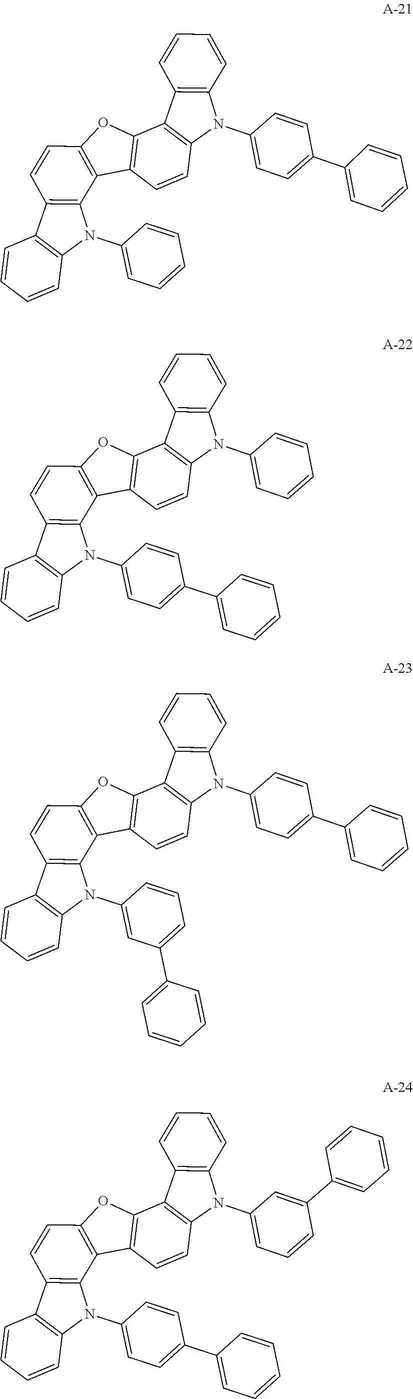 [SMILES: c1ccc(-c2ccc(-n3c4ccccc4c4c5oc6ccc7c8ccccc8n(-c8ccccc8)c7c6c5ccc43)cc2)cc1 ; c1ccc(-c2ccc(-n3c4ccccc4c4ccc5oc6c(ccc7c6c6ccccc6n7-c6ccccc6)c5c43)cc2)cc1 ; c1ccc(-c2ccc(-n3c4ccccc4c4c5oc6ccc7c8ccccc8n(-c8cccc(-c9ccccc9)c8)c7c6c5ccc43)cc2)cc1 ; c1ccc(-c2ccc(-n3c4ccccc4c4ccc5oc6c(ccc7c6c6ccccc6n7-c6cccc(-c7ccccc7)c6)c5c43)cc2)cc1]

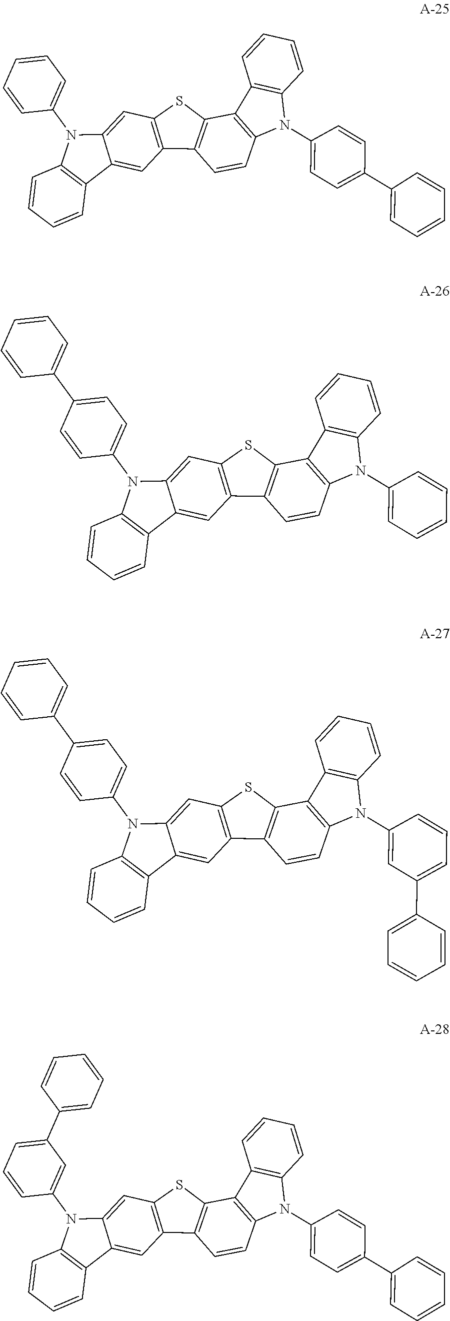 [SMILES: c1ccc(-c2ccc(-n3c4ccccc4c4c5sc6cc7c(cc6c5ccc43)c3ccccc3n7-c3ccccc3)cc2)cc1 ; c1ccc(-c2ccc(-n3c4ccccc4c4cc5c(cc43)sc3c5ccc4c3c3ccccc3n4-c3ccccc3)cc2)cc1 ; c1ccc(-c2ccc(-n3c4ccccc4c4cc5c(cc43)sc3c5ccc4c3c3ccccc3n4-c3cccc(-c4ccccc4)c3)cc2)cc1 ; c1ccc(-c2ccc(-n3c4ccccc4c4c5sc6cc7c(cc6c5ccc43)c3ccccc3n7-c3cccc(-c4ccccc4)c3)cc2)cc1]

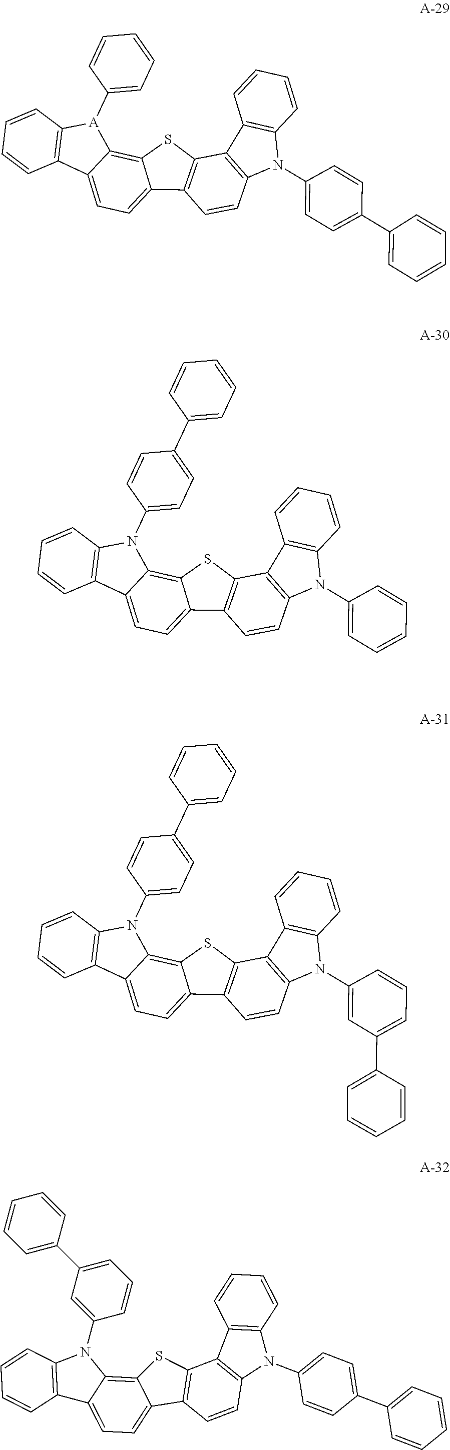

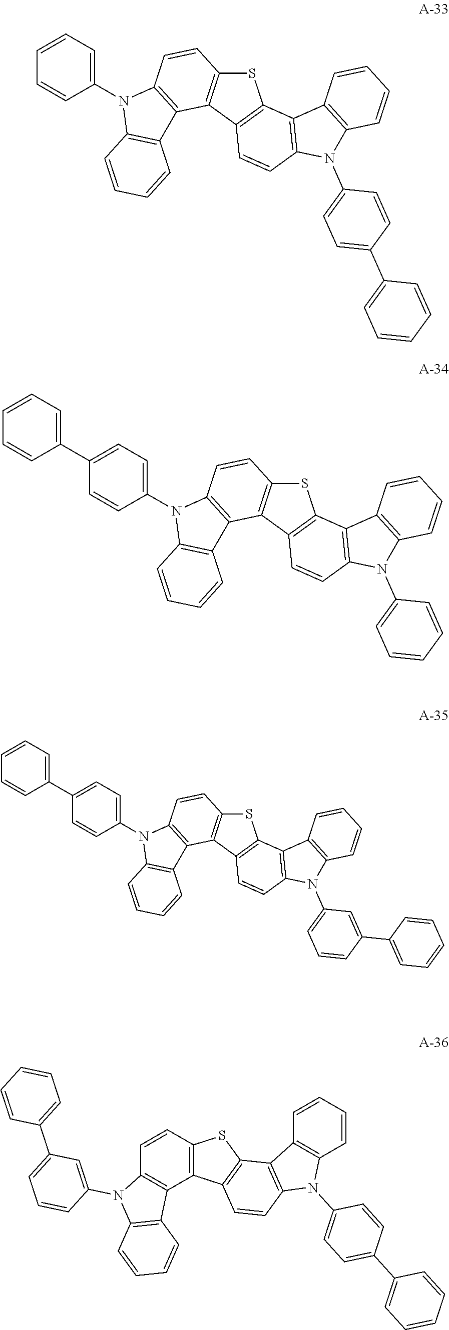 [SMILES: c1ccc(-c2ccc(-n3c4ccccc4c4c5sc6ccc7c(c8ccccc8n7-c7ccccc7)c6c5ccc43)cc2)cc1 ; c1ccc(-c2ccc(-n3c4ccccc4c4c5c(ccc43)sc3c5ccc4c3c3ccccc3n4-c3ccccc3)cc2)cc1 ; c1ccc(-c2ccc(-n3c4ccccc4c4c5c(ccc43)sc3c5ccc4c3c3ccccc3n4-c3cccc(-c4ccccc4)c3)cc2)cc1 ; c1ccc(-c2ccc(-n3c4ccccc4c4c5sc6ccc7c(c8ccccc8n7-c7cccc(-c8ccccc8)c7)c6c5ccc43)cc2)cc1]

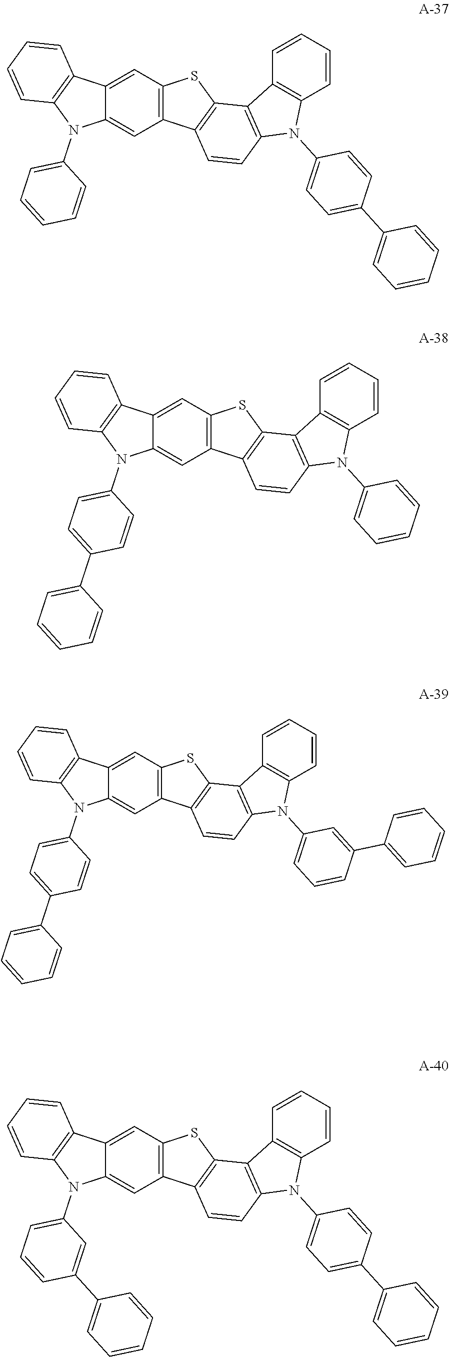 [SMILES: c1ccc(-c2ccc(-n3c4ccccc4c4c5sc6cc7c8ccccc8n(-c8ccccc8)c7cc6c5ccc43)cc2)cc1 ; c1ccc(-c2ccc(-n3c4ccccc4c4cc5sc6c(ccc7c6c6ccccc6n7-c6ccccc6)c5cc43)cc2)cc1 ; c1ccc(-c2ccc(-n3c4ccccc4c4cc5sc6c(ccc7c6c6ccccc6n7-c6cccc(-c7ccccc7)c6)c5cc43)cc2)cc1 ; c1ccc(-c2ccc(-n3c4ccccc4c4c5sc6cc7c8ccccc8n(-c8cccc(-c9ccccc9)c8)c7cc6c5ccc43)cc2)cc1]

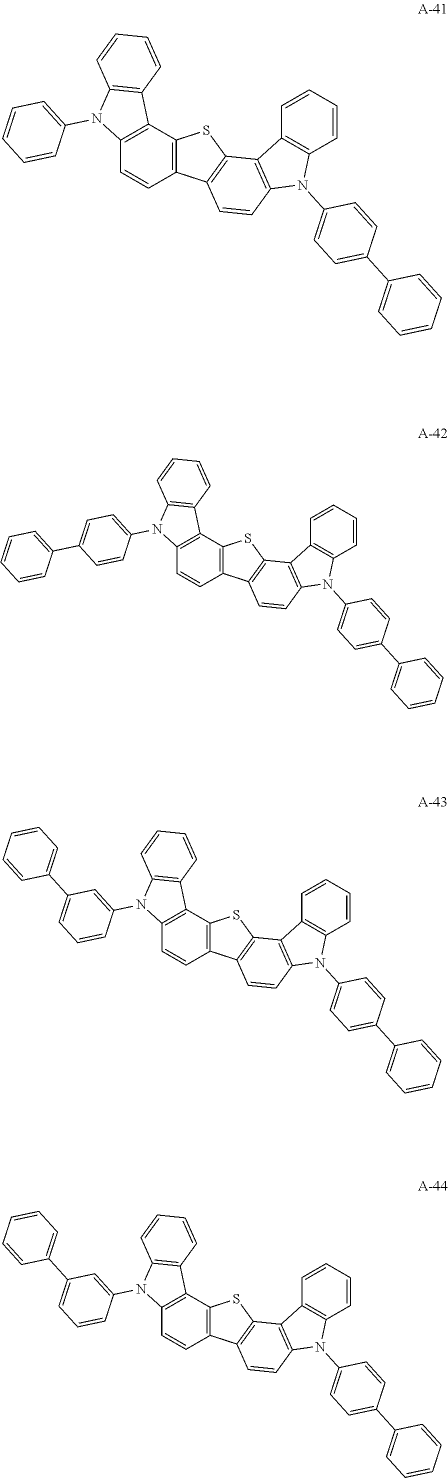 [SMILES: c1ccc(-c2ccc(-n3c4ccccc4c4c5sc6c(ccc7c6c6ccccc6n7-c6ccccc6)c5ccc43)cc2)cc1 ; c1ccc(-c2ccc(-n3c4ccccc4c4c5sc6c(ccc7c6c6ccccc6n7-c6ccc(-c7ccccc7)cc6)c5ccc43)cc2)cc1 ; c1ccc(-c2ccc(-n3c4ccccc4c4c5sc6c(ccc7c6c6ccccc6n7-c6cccc(-c7ccccc7)c6)c5ccc43)cc2)cc1 ; c1ccc(-c2ccc(-n3c4ccccc4c4c5sc6c(ccc7c6c6ccccc6n7-c6cccc(-c7ccccc7)c6)c5ccc43)cc2)cc1]

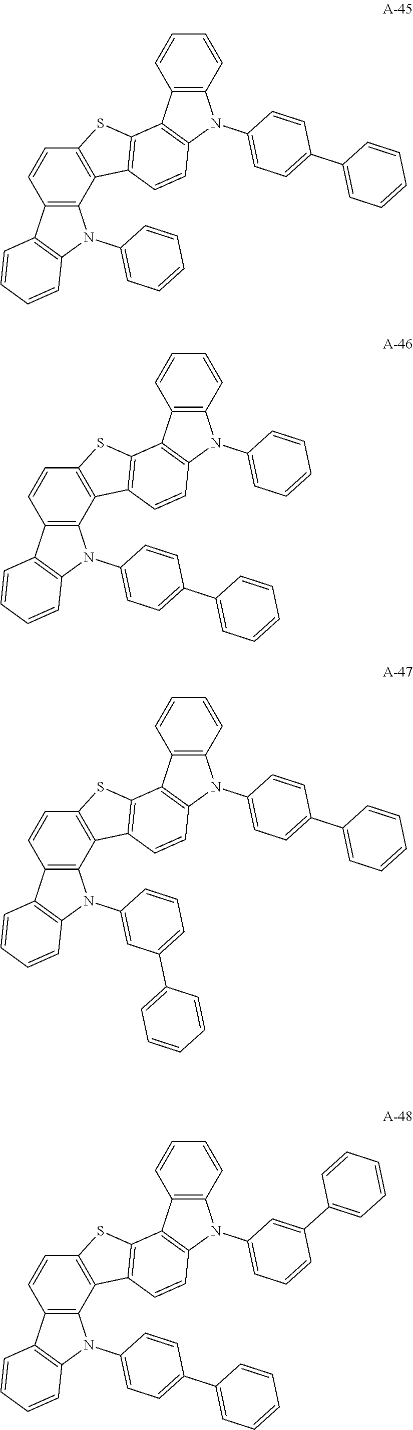 [SMILES: c1ccc(-c2ccc(-n3c4ccccc4c4c5sc6ccc7c8ccccc8n(-c8ccccc8)c7c6c5ccc43)cc2)cc1 ; c1ccc(-c2ccc(-n3c4ccccc4c4ccc5sc6c(ccc7c6c6ccccc6n7-c6ccccc6)c5c43)cc2)cc1 ; c1ccc(-c2ccc(-n3c4ccccc4c4c5sc6ccc7c8ccccc8n(-c8cccc(-c9ccccc9)c8)c7c6c5ccc43)cc2)cc1 ; c1ccc(-c2ccc(-n3c4ccccc4c4ccc5sc6c(ccc7c6c6ccccc6n7-c6cccc(-c7ccccc7)c6)c5c43)cc2)cc1]

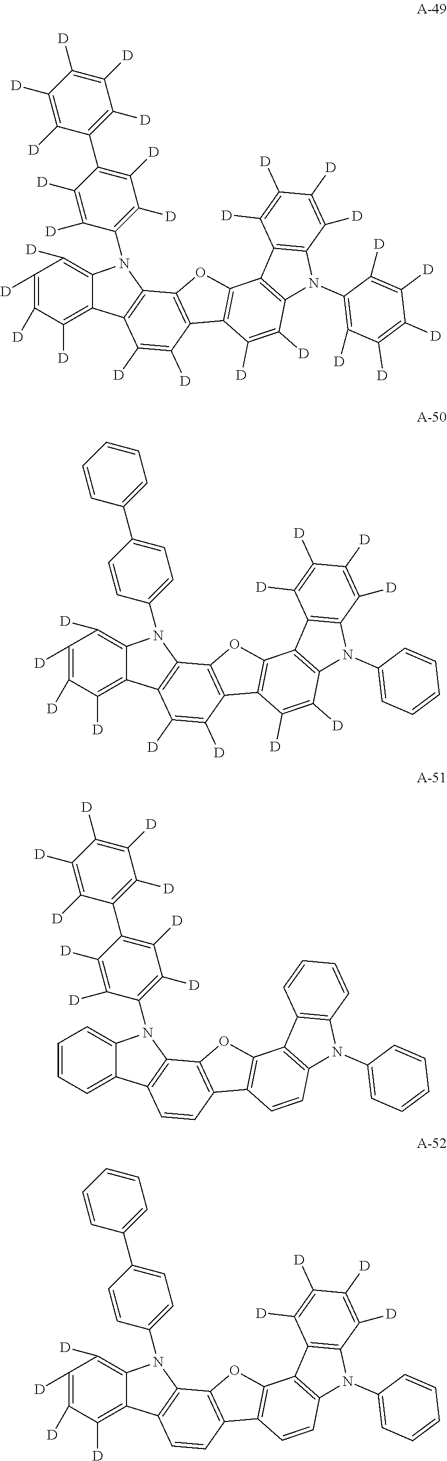

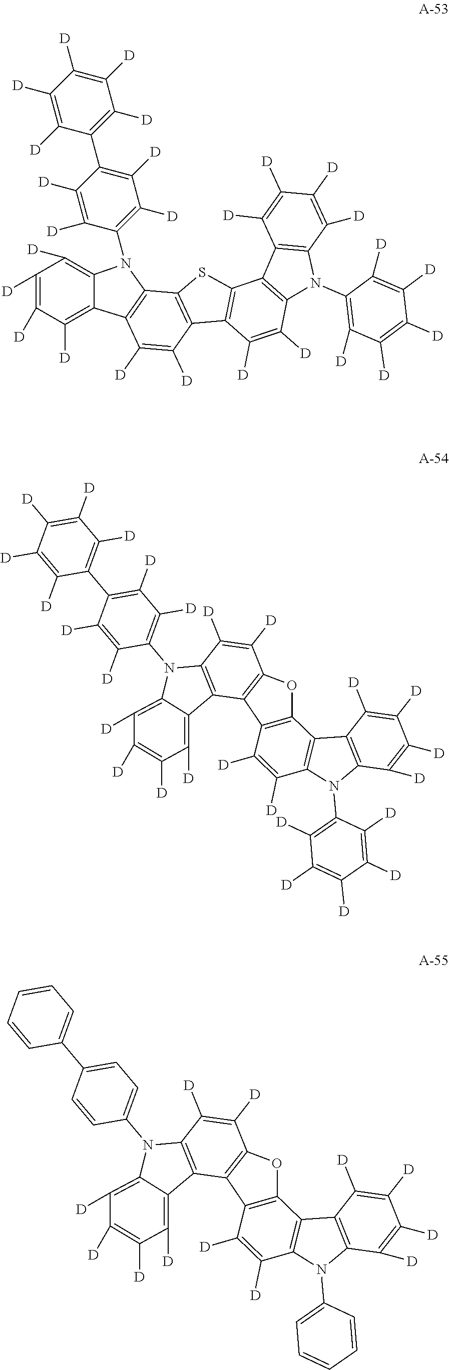

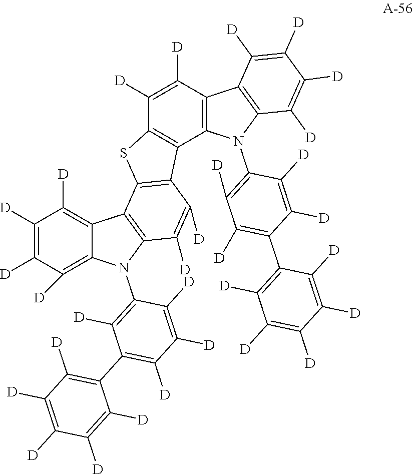 [SMILES: [2H]c1c([2H])c([2H])c(-c2c([2H])c([2H])c(-n3c4c([2H])c([2H])c([2H])c([2H])c4c4c([2H])c([2H])c5sc6c(c([2H])c([2H])c7c6c6c([2H])c([2H])c([2H])c([2H])c6n7-c6c([2H])c([2H])c([2H])c(-c7c([2H])c([2H])c([2H])c([2H])c7[2H])c6[2H])c5c43)c([2H])c2[2H])c([2H])c1[2H]]

7. The composition for an organic optoelectronic device of claim 1, wherein:
the second compound is represented by Chemical Formula 3-I or Chemical Formula 3-II: 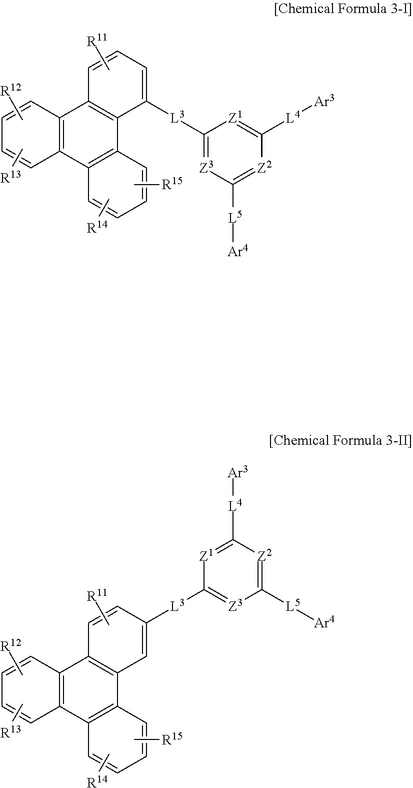
in Chemical Formula 3-I or Chemical Formula 3-II, Z¹ to Z³, L³ to L⁵, Ar³, Ar⁴ and R¹¹ to R¹⁵ are defined the same as those of Chemical Formula 3.

8. The composition for an organic optoelectronic device of claim 1, wherein Ar³ and Ar⁴ are each independently a substituted or unsubstituted phenyl group, a substituted or unsubstituted biphenyl group, or a substituted or unsubstituted naphthyl group.

9. The composition for an organic optoelectronic device of claim 1, wherein:
Ar³ and Ar⁴ are each independently a group of Group II: 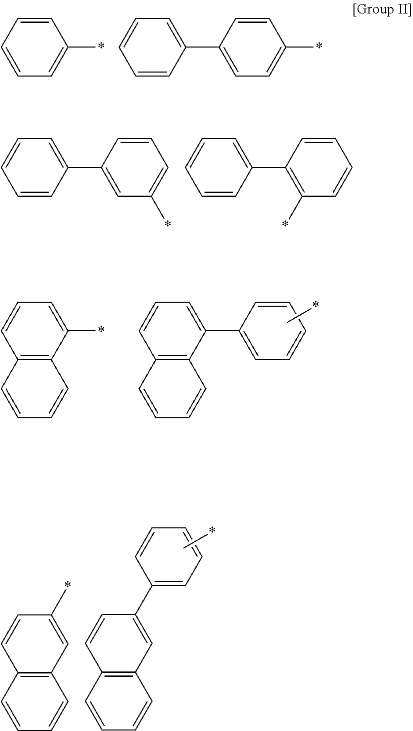
in Group II, * is a linking point.

10. The composition for an organic optoelectronic device of claim 1, wherein the second compound is is a compound of Group 2:

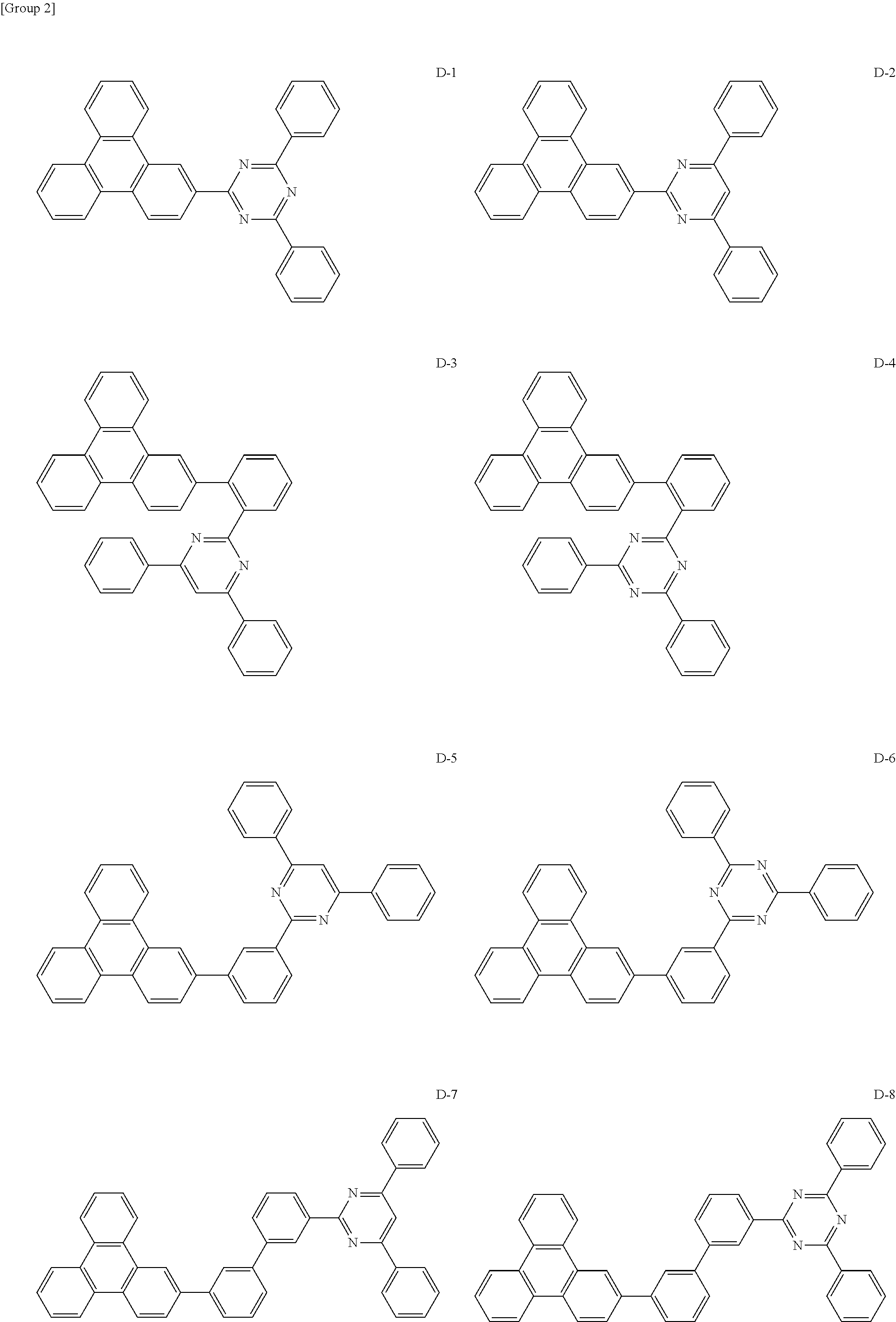

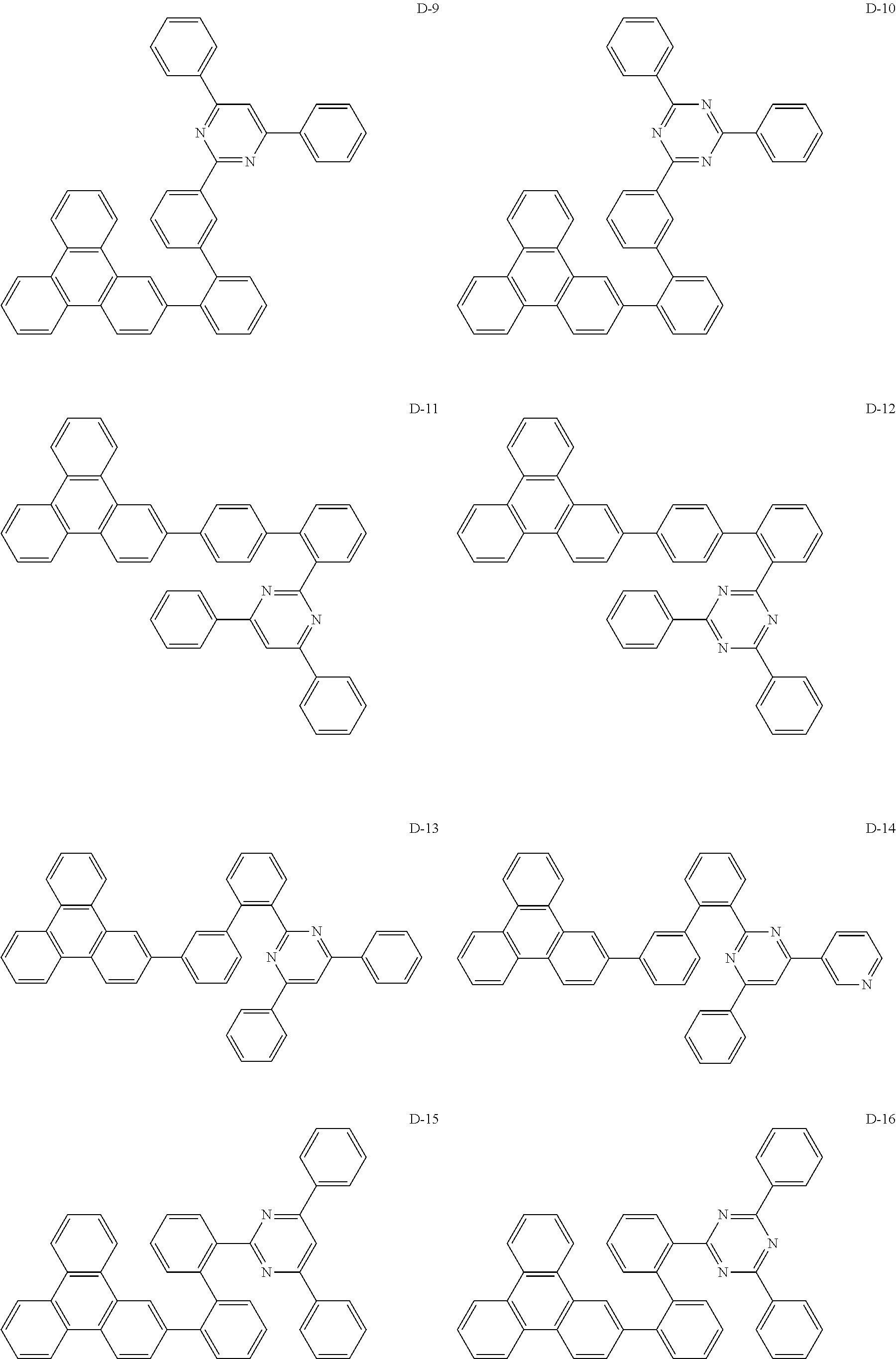

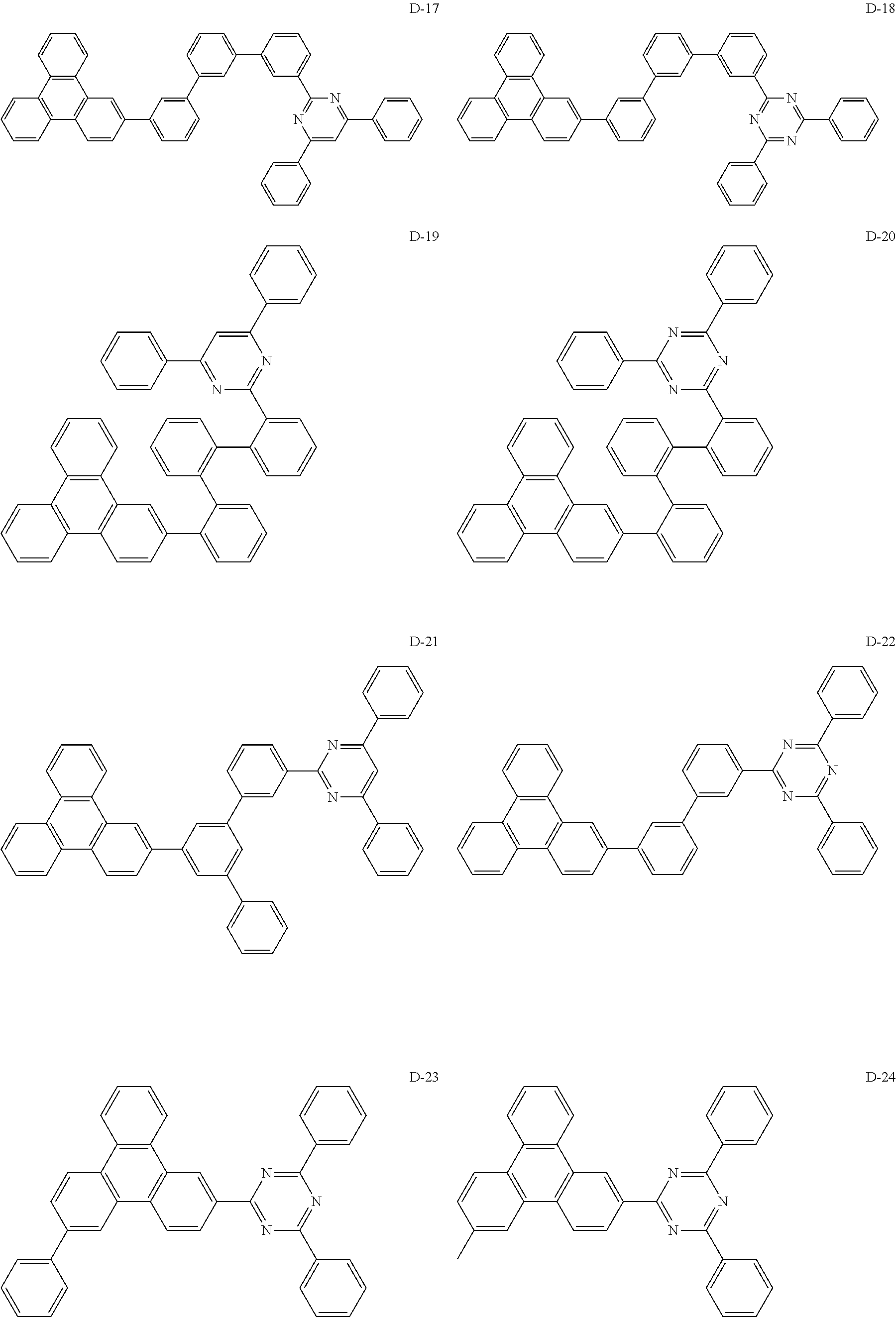

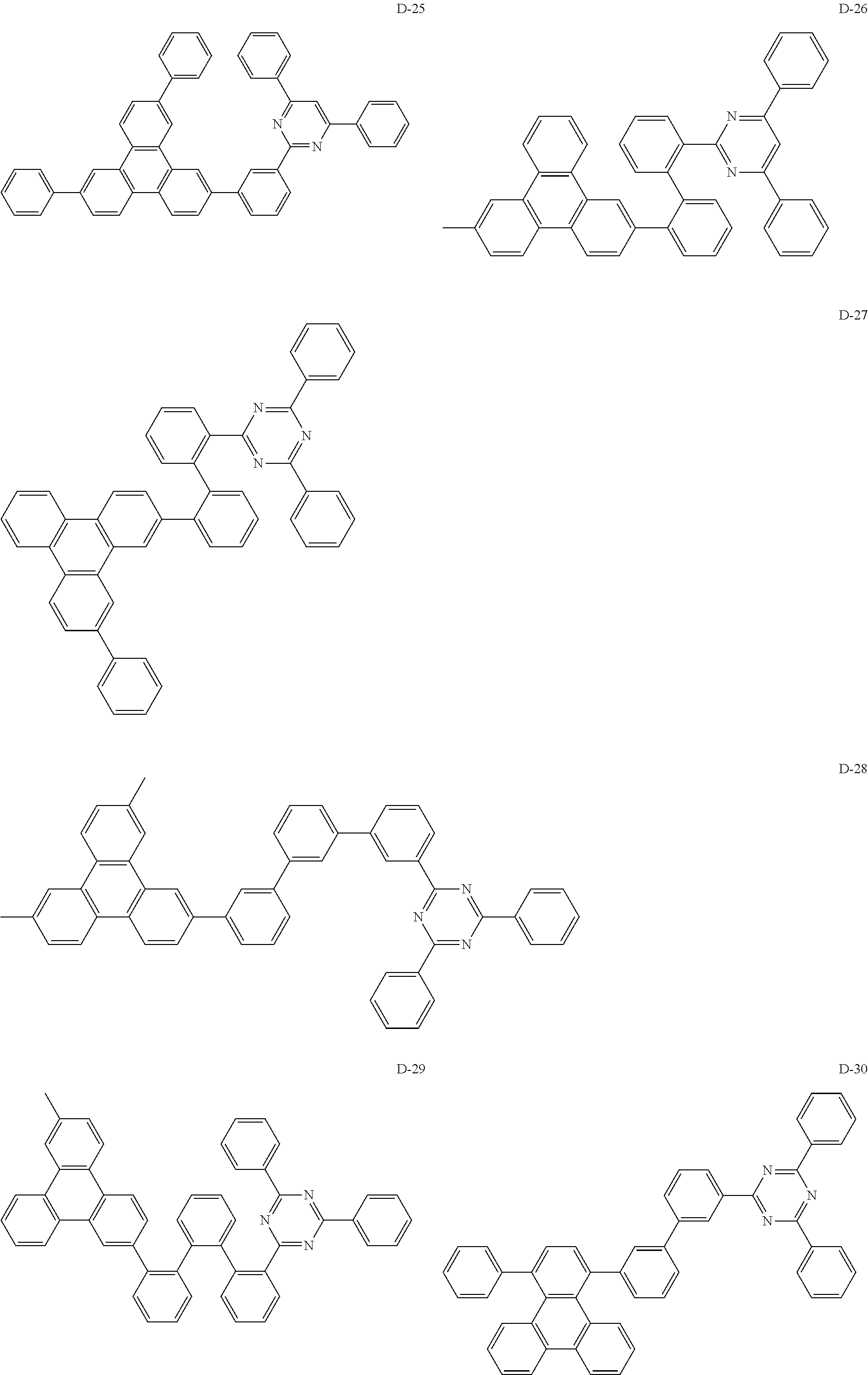

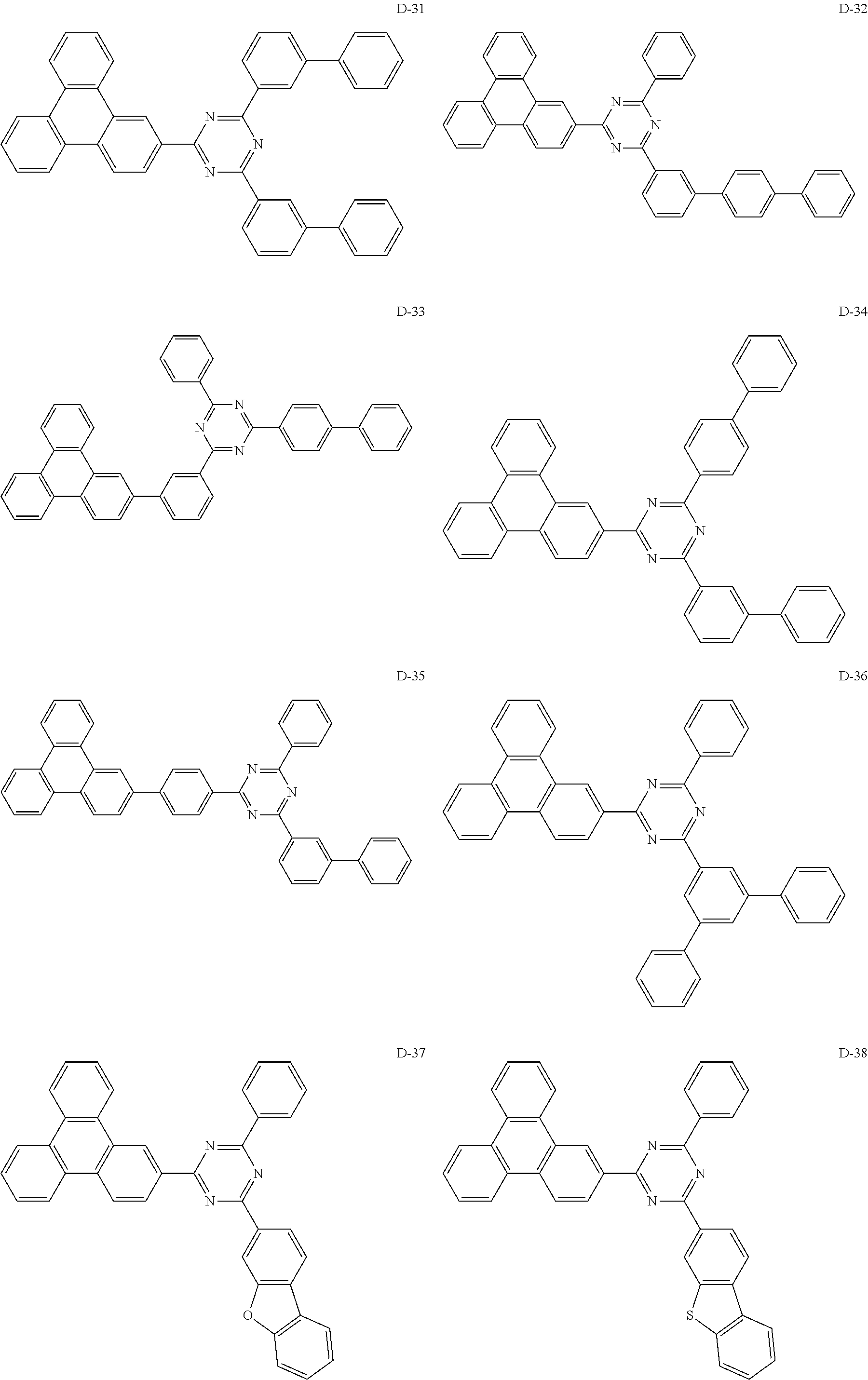

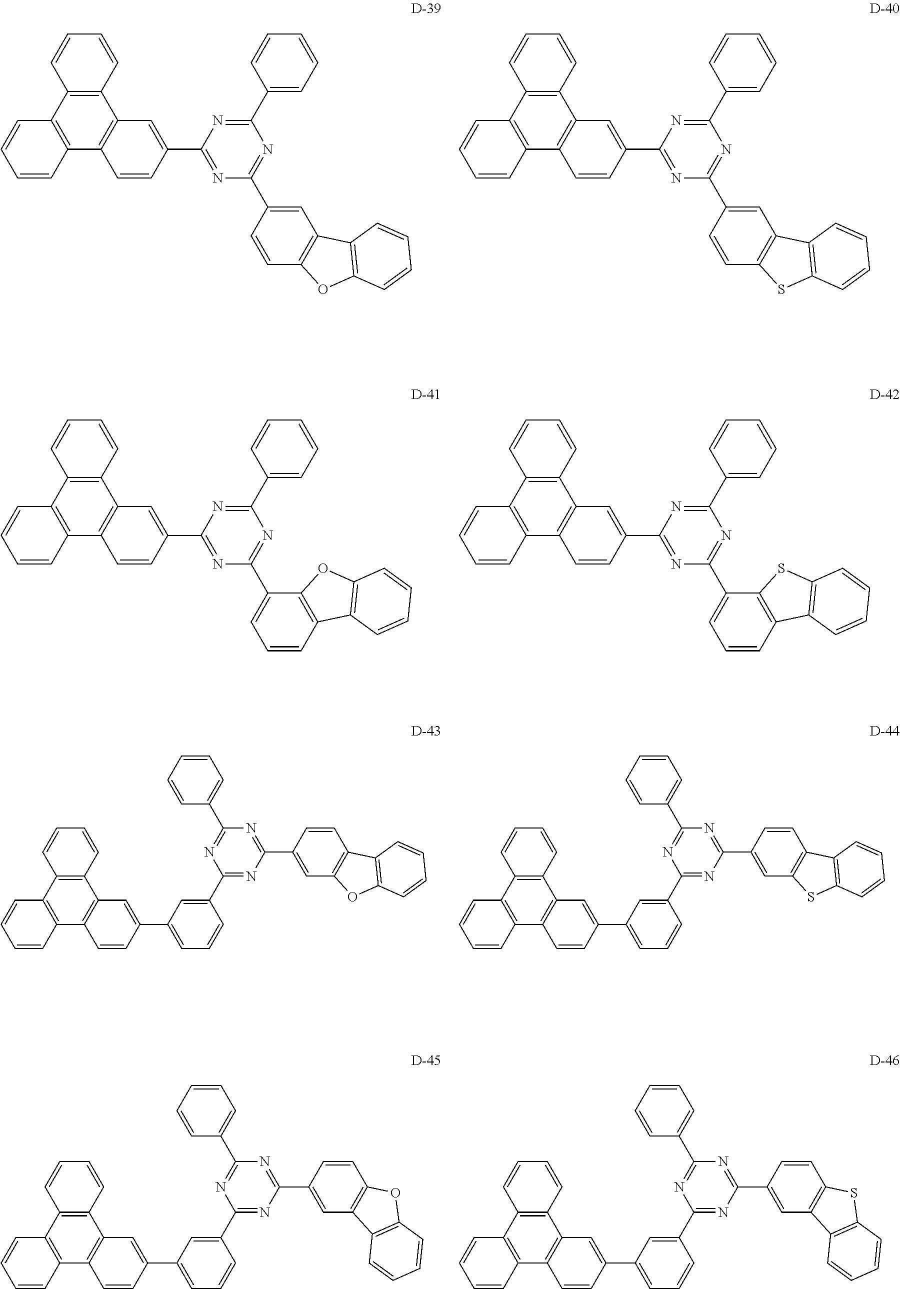

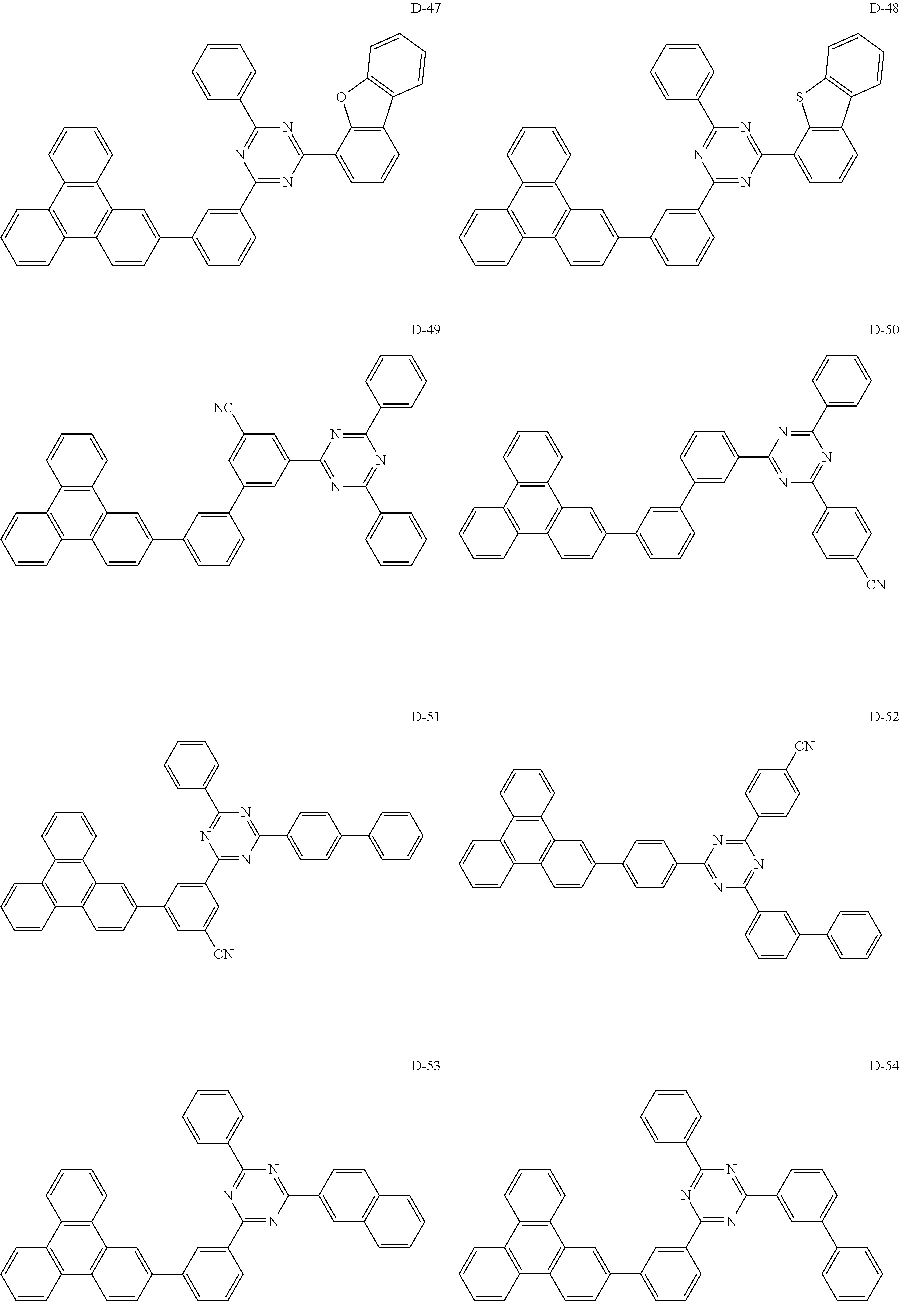

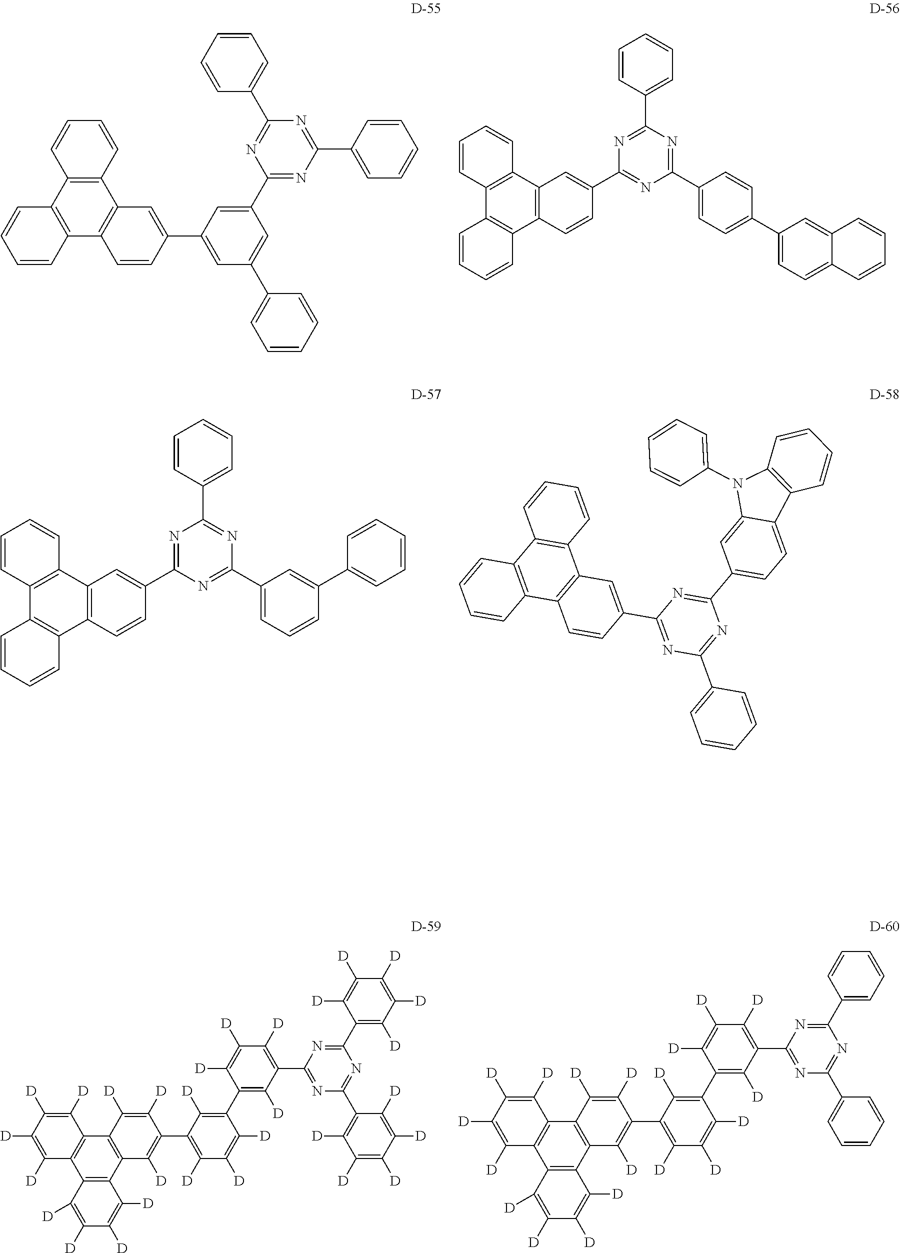

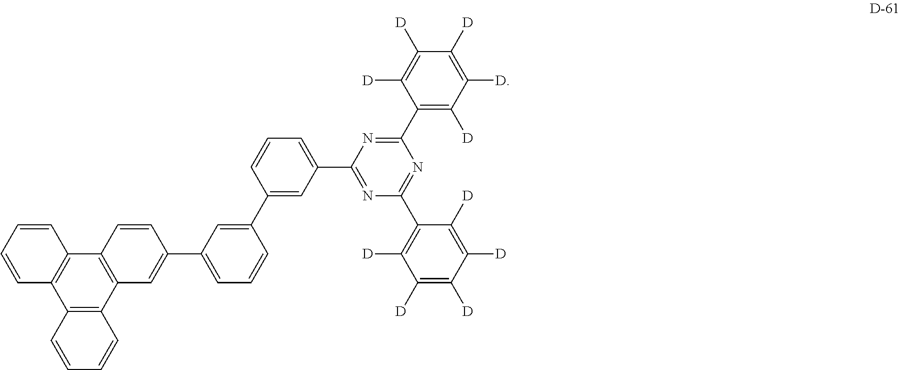

11. The composition for an organic optoelectronic device of claim 1, wherein:
the first compound is represented by Chemical Formula 1A or Chemical Formula 1B, and
the second compound is represented by Chemical Formula 3-II: 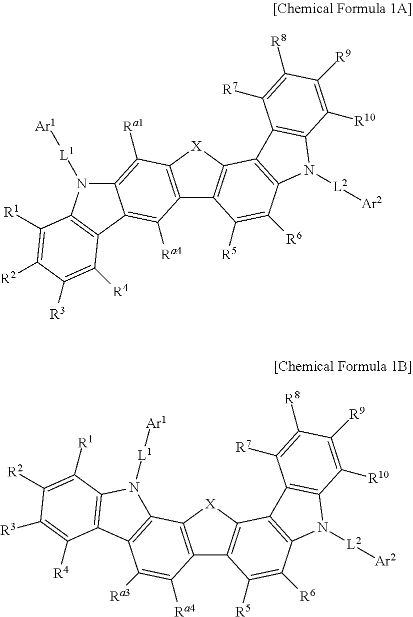
in Chemical Formula 1A and Chemical Formula 1B,
Ar¹ and Ar² are each independently a substituted or unsubstituted phenyl group, a substituted or unsubstituted biphenyl group, or a substituted or unsubstituted naphthyl group,
L¹ and L² are each independently a single bond or a substituted or unsubstituted phenylene group,
R¹ to R¹⁰, R^{a1}, R^{a3} and R^{a4} are each independently hydrogen, deuterium, a cyano group, a halogen, a substituted or unsubstituted C1 to C10 alkyl group or a substituted or unsubstituted C6 to C12 aryl group, and
X is O or S; 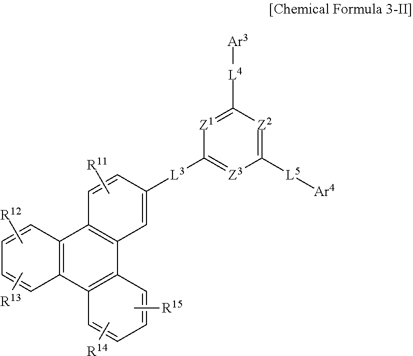
in Chemical Formula 3-II,
Z¹ to Z³ are each N,
Ar³ and Ar⁴ are each independently a substituted or unsubstituted phenyl group, a substituted or unsubstituted biphenyl group, or a substituted or unsubstituted naphthyl group,
L³ to L⁵ are each independently a single bond or a substituted or unsubstituted phenylene group, and
R¹¹ to R¹⁵ are each independently hydrogen, deuterium, a cyano group, a halogen, a substituted or unsubstituted C1 to C10 alkyl group or a substituted or unsubstituted C6 to C12 aryl group.

12. An organic optoelectronic device, comprising
an anode and a cathode facing each other, and
at least one organic layer disposed between the anode and the cathode,
wherein the organic layer includes a light emitting layer, and
the at least one light emitting layer includes the composition for an organic optoelectronic device of claim 1.

13. The organic optoelectronic device of claim 12, wherein the composition for an organic optoelectronic device is a host of the at least one light emitting layer.

14. A display device comprising the organic optoelectronic device of claim 12.
